(19) United States
(12) Patent Application Publication
(10) Pub. No.: US 2025/0297307 A1
(43) Pub. Date: Sep. 25, 2025

(54) MASS SPECTOMETRY-BASED DIRECT SEQUENCING OF TRANSFER RNAS DE NOVO AND QUANITATIVE MAPPING OF MULTIPLE RNA MODIFICATIONS

(71) Applicant: New York Institute of Technology, Old Westbury, NY (US)

(72) Inventor: Shenglong Zhang, Woodcliff Lake, NJ (US)

(21) Appl. No.: 18/912,187

(22) Filed: Oct. 10, 2024

Related U.S. Application Data
(60) Provisional application No. 63/589,129, filed on Oct. 10, 2023.

(51) Int. Cl. C12Q 1/6872; G01N 30/72; G01N 30/86; H01J 49/00

(52) U.S. Cl. CPC C12Q 1/6872; G01N 30/7233; G01N 30/8675; H01J 49/0036

(57) ABSTRACT

The present disclosure provides a novel de novo sequencing method (herein referred to as MLC-Seq) of cellular RNAs within a sample including unbiased sequencing of nucleotide modifications, while also identifying site-specific stoichiometry of partial modifications. In one aspect, the method is used to sequence tRNAs and tRNA modifications within a mixed RNA sample.

### CROSS REFERENCE TO RELATED APPLICATIONS

This application claims the benefit of and priority to U.S. Provisional Application No. 63/589,129, filed on Oct. 10, 2023, the entire contents of which being incorporated by reference herein in their entireties.

### GOVERNMENT SUPPORT STATEMENT

This invention was made with government support under R01HG012853 awarded by the National Institutes of Health. The government has certain rights in the invention.

### REFERENCE TO ELECTRONIC SEQUENCE LISTING

The application contains a Sequence Listing which has been submitted electronically in XML format and is hereby incorporated by reference in its entirety. Said .XML copy, created on Jun. 16, 2025, is named “2637-6.xml” and is 234,070 bytes in size. The sequence listing contained in this .XML file is part of the specification and is hereby incorporated by reference herein in its entirety.

### TECHNICAL FIELD

The present disclosure provides a novel de novo sequencing method (herein referred to as MLC-Seq) of cellular RNAs within a sample including unbiased sequencing of nucleotide modifications, while also identifying site-specific stoichiometry of partial modifications. In one aspect, the method is used to sequence tRNAs and tRNA modifications within a mixed RNA sample.

### BACKGROUND

Despite the widespread utilization of high-throughput next-generation sequencing (NGS), the ‘true’ sequence of RNA, i.e., the identity and location of every nucleotide (canonical or modified) within a full-length RNA₁, remains unknown due to the lack of a general method for direct sequencing of any nucleotide (modified or not) at single-nucleotide resolution. NGS-based RNA sequencing methods do not sequence RNA directly but rather its complementary DNA (cDNA), which contains canonical nucleotides only. To sequence modified RNA nucleotides, these NGS-based methods require additional specific procedures²⁻⁸. Only a small number of the over 170 known modified nucleotides can be identified by NGS sequencing, making this approach inefficient for modification-rich tRNAs. Other efforts have used direct nanopore-based sequencing for mapping modifications in long RNAs⁹⁻¹³ and sequencing tRNAs₁₄ but suffered from high error rates^{1,5}. Furthermore, RNA samples often contain coexisting isoforms, molecules that are nominally of the same RNA sequence but that have different compositions. These arise from partial nucleotide modifications or alterations; those present in less than 100% of the molecules of a given RNA sequence. Quantifying the stoichiometries of these site-specific partial modifications remains challenging¹⁵. Accordingly, novel methods for sequencing of RNA molecules are needed.

### SUMMARY

The current disclosure is related a novel MS ladder RNA sequencing method, referred to herein as MLC-SEQ, which can be used to directly sequence RNA, without the need for prior cDNA synthesis, to simultaneously determine the nucleotide sequence of an RNA molecule with single nucleotide resolution and reveal the presence, type, location and quantity of different nucleotide modifications that the RNA molecule carries. The provided MLC-SEQ sequencing method addresses incomplete ladder issues associated with MS ladder sequencing of cellular RNAs by providing a number of processing steps that can be used to address and resolve said issues. Such techniques can be used advantageously to correlate the biological functions of any given RNA molecule with its associated modifications and for quality control of RNA-based therapeutics.

Specifically, the method identifies RNA sequences and their variants, providing a powerful approach to sequence full-length RNAs, including their modifications, even in the presence of other RNAs. MLC-Seq effectively reveals nucleotide identities, partial modification stoichiometry of multiple RNA modifications. Such a method includes observation in RNA modification changes following AlkB enzymatic treatment.

To manage the LC-MS data complexity of controlled acid hydrolyzed yeast total tRNA samples, the present disclosure provides a MLC-Seq platform that employs three functionalities, including: (1) de novo sequencing (without sequence input) to identify tRNA types present in the samples, (2) site-specific mapping of tRNA modifications by cross referencing between tRNA database sequences and the LC-MS data⁴³ and (3) site-specific quantification of partial tRNA modification stoichiometry.

The provided MLC-Seq platform can monitor changes in RNA modification dynamics and map modifications that have altered stoichiometry in different cellular and disease contexts. For example, the presence of a branch point in a sigmoidal fragment ladder curve on a tR mass plot is indicative of a partial modification/editing. In contrast to cDNA-based RNA sequencing, which removes information on modifications, MLC-Seq preserves information regarding tRNA sample diversity (for visualizing each tRNA) and modification (for revealing modification type, location, stoichiometry, etc.).

The present disclosure provides a method for de novo sequencing of tRNAs and site-specific quantification of RNA modification stoichiometries. The method comprises the step of starting with a tRNA sample for sequencing that is divided into two samples. One sample in referred to as intact (no acid hydrolysis) while the other sample is subjected to controlled acid hydrolysis. The method further comprises the step of direct observation of partial nucleotide modifications or editing in the intact sample. The method further comprises the step of conducting MS ladder sequencing of the RNA sample that had been subjected to controlled acid hydrolysis. In the RNA sample subjected to controlled acid hydrolysis the RNA is converted into two series of ladders (5′ and 3′) composed of a series of fragments of varying lengths for MS ladder sequencing. After acid degradation, the 5′ and 3′ ladders display sigmoidal curves on a tR-mass plot, where branches in the plot indicate the position and types of partially modified or edited nucleotides. In this step, de novo base calling of the complete sequence of the tRNA isoforms, may be accomplished using novel algorithms that identify and separate each tRNA species or isoform's MS ladders from LC-MS data. As another step in the method, site-specific quantification of stoichiometry for partial tRNA nucleotide modifications and editing is accomplished using data from both intact and ladder levels. EIC peak areas of each fragment indicates the stoichiometry ratio, e.g., modification of the tRNA at a given position. Ladder level quantification aligns with relative abundances at the intact level, confirming initially observed modifications or editing.

In an embodiment the de novo base calling of the complete sequence of the tRNA may be accomplished using one or more of the following algorithms: (i) a Homology Searching before, or after, fragmentation for identification of related RNA isoforms; (ii) a MassSum algorithm which identifies and isolates the 3′, 5′ ladder fragments as well as other related fragments; (iii) a GapFill algorithm to complement MassSum; and (iv) a Ladder Complementation algorithm.

In an embodiment of the provided method, a computer-implemented sequencing method is employed for (i) identifying RNA isoforms based on a homology search function configured to divide the intact RNA molecules into two or more groups with each group representing one specific RNA species and its related isoforms (ii) determining the Mass Sum of any of two fragments including but not limited to 3′/5′ ladder fragments; (iii) for the step of determining if any of the two ladder fragments cannot pair based on the mass sum value for a given RNA, and if so finding one of them by use of a GapFill algorithm, configured to search for ladder fragments missed by MassSum determination; and/or (iv) for completion of incomplete ladders (after MassSum and GapFilling processing) using other related isoforms (identified through homology searches) to obtain a more complete ladder for sequencing.

The present disclosure provides a kit for use in generating the sequence of one or more RNA molecules and detecting the presence, identity, location, and quantity of RNA nucleotide modifications on said one or more RNA molecules, said kit comprising one or more components for performance of a method comprising one or more of the steps of (i) controlled fragmentation of the RNA to form sequencable ladder fragments such as 5′ and 3′ MS ladder fragments; (ii) mass measurement of resultant degraded RNA samples containing RNAs and their fragmented fragments; and (iii) data processing, including identification and separation of 3′ and/or 5′ MS ladder fragments thereby generating the sequence of one or more RNA molecules and detecting the presence, identity, location, and quantity of RNA nucleotide modifications.

In another embodiment an MS based sequencing instrument is provided for use in generating the sequence of one or more RNA molecules and detecting the presence, identity, location, and quantity of RNA nucleotide modifications on said one or more RNA molecules, said instrument comprising one or more components for performance of the method comprising the steps of (i) controlled fragmentation of the RNA to form sequencable ladder fragments such as 5′ and 3′ MS ladder fragments; (ii) mass measurement of resultant degraded RNA samples containing RNAs and their fragmented fragments; and (iii) data processing, including identification and separation of 3′ and/or 5′ MS ladder fragments thereby generating the sequence of one or more RNA molecules and detecting the presence, identity, location, and quantity of RNA nucleotide modifications.

Provided herein is a non-transitory computer-readable medium storing instructions that, when executed by a processor, cause the processor to perform methods for generating the sequence of one or more RNA molecules and detecting the presence, identity, location, and quantity of RNA nucleotide modifications on said one or more RNA molecules, said method RNA comprising the steps of (i) controlled fragmentation of the RNA to form 5′ and 3′ MS ladder fragments; (ii) mass measurement of resultant degraded RNA samples containing RNAs and their fragmented fragments; and (iii) data processing, including identification and separation of 3′ and/or 5′ MS ladder fragments thereby generating the sequence of one or more RNA molecules and detecting the presence, identity, location, and quantity of RNA nucleotide modifications.

Further details and aspects of exemplary embodiments of the disclosure are described in more detail below with reference to the appended figures. Any of the above aspects and embodiments of the disclosure may be combined without departing from the scope of the disclosure.

### BRIEF DESCRIPTION OF THE DRAWINGS

This patent or application file contains at least one drawing executed in color. Copies of this patent or patent application publication with color drawing(s) will be provided by the Office upon request and payment of the necessary fee. Various embodiments of methods are described herein with reference to the drawings wherein:

FIG. 1A-F. Schematic illustration of MLC-Seq workflow for de novo sequencing of tRNAs and site-specific quantification of RNA modification stoichiometries. (FIG. 1A) A tRNA sample for MLC-Seq: ˜ 10% is analyzed intact, while the remaining 90% is subject to controlled acid hydrolysis. (FIG. 1B) Direct observation of partial nucleotide modifications or editing at the intact level. For example, a 14 Da mass difference between tRNA isoforms IF1 and IF3 suggests a partial methylation (CH2), while a 16 Da mass difference between IF3 and IF4 indicate a potential partial editing A-G, with one oxygen atom difference denoted as “Ox”. (FIG. 1C) Controlled acid hydrolysis coverts the intact RNA into two series of ladders (5′ and 3′) composed of a series of fragments of varying lengths for MS ladder sequencing. (FIG. 1D) After acid degradation, the 5′ and 3′ ladders display sigmoidal curves on a tR-mass plot, where branches in the plot indicate the position and types of partially modified or edited nucleotides. For example, the 14 Da difference between the branched ladder fragment and original one at the same position indicates partial methylation. The branch's starting point identifies the partial modification's location at position 58 of the tRNA. (FIG. 1E) De novo base calling of the complete sequence of tRNA isoform IF1, using novel algorithms that identify and separate each tRNA species or isoform's MS ladders from LC-MS data. (SEQ ID NO. 1) (FIG. 1F) Site-specific quantification of stoichiometry for partial tRNA nucleotide modifications and editing using data from both intact and ladder levels. EIC peak areas of each fragment indicates the stoichiometry ratio, e.g., m1A vs A at position 58 of the tRNA (d). Ladder level quantification aligns with relative abundances at the intact level, confirming initially observed modifications or editing (b).

FIG. 2A-F. MLC-Seq De novo sequencing of yeast-derived tRNA-Phe using algorithms of homology search, MassSum and ladder complementation. (FIG. 2A) Homology search results before acid degradation. Here, C and A indicate that the mass difference between two related tRNA isoforms, 305.0413 and 329.0525 Da, can be attributed to the nucleotides C and A, respectively. “Ox” indicates that the mass difference (15.9949 Da) between two related tRNA isoforms can be from an oxygen atom. (FIG. 2B) Illustration of the principle that enables MassSum to function: after a controlled single cleavage of a phosphodiester bond in the RNA strand, the combined mass of the two resulting fragments must be equal to the mass of the original RNA plus the mass of a water molecule. (FIG. 2C) Example of 5′ and 3′ ladders and the use of MassSum to identify pairs of linked fragments. (FIG. 2D) Sequencing of yeast-derived tRNA-Phe, using LC-MS deconvoluted data and the MassSum algorithm to identify ladder fragments of Phe IF1 with an intact monoisotopic mass of 24252.2692 Da. Together with other MLC-Seq algorithms, MassSum separates ladder fragments of each tRNA isoform, allowing for complete de novo sequencing. Inset shows the masses of the intact isoforms and their relative abundances. (FIG. 2E) The identified nucleotides for five tRNA-Phe isoforms after ladder complementation. The depth information on the bottom indicates the number of times each nucleotide was read independently from the tRNA isoform ladders. (FIG. 2F) The complete sequences for the five isoforms, along with a reference sequence obtained in a previous work.²¹ The nucleotides highlighted in red differ from the full-length isoform Phe IF2. The sequencing result of tRNA-Phe reveals partial nucleotide modifications/editing as well as terminal truncations for five different isoforms. (SEQ ID NO. 2-5)

FIG. 3A-F. Workflow for processing of MLC-Seq data. (FIG. 3A) Initial data after deconvolution of the LC-MS raw data file. (FIG. 3B) Initial partial ladders after application of the MassSum algorithm. (FIG. 3C) GapFill identifies additional nucleotides (empty circles) in each ladder missed by the MassSum data separation step when neither half of a 5′/3′fragment pair was found. (FIG. 3D) Data is separated into distinct 5′ and 3′ ladders for each isoform. (FIG. 3E) Ladder complementation combines partial results across multiple 5′ or 3′ ladders, filling sequence gaps in one isoform using ladder fragment data from a related one. (FIG. 3F) Cross-ladder complementation, based on 5′ and 3′ ladders being the reverse of each other, can be applied to any remaining sequence gaps to obtain a complete sequence, a complete workflow is detailed in FIG. 7.

FIG. 4A-D. Quantifying stoichiometries for partial nucleotide modifications/editing. (FIG. 4A) t_{R}-mass data of tRNA-Glu output from LC-MS raw data. (FIG. 4B) Close-up view of t_{R}-mass data for tRNA-Glu showing a branch in the ladder at position 58 revealing a partial modification or alteration at this position; the mass difference of ˜14 Da between the two branches specifically indicates a partial methylation. A 3′-ladder fragment is missed at position 55 because the 2′-O methyl group of m⁵Um at position 54 of tRNA-Glu blocks formic acid hydrolysis of the phosphodiester bond between these two positions. (FIG. 4C) EICs of 3′-ladder fragments containing mA (top, light blue, negative charge states 8-9) and canonical A (bottom, dark blue, negative charge states 9-10) at postbranch position 58. Only the peak areas of ladder fragments with single identical charge state (-9) were used to calculate the stoichiometry of canonical A and mA at this position. (FIG. 4D) Table of subsequent ladder fragment masses, integrated peak areas, retention times, and compositions from the partial m¹A methylation at position 58.

FIG. 5A-D. Using MLC-Seq to de novo sequence tRNA-Glu derived from mouse liver and track modification changes after AlkB treatment. (FIG. 5A) Complete sequence of tRNA-Glu. (SEQ ID NO. 6) (FIG. 5B) Position and stoichiometry of the noncanonical nucleotides and partially modified nucleotides in tRNA-Glu. (FIG. 5C) Stoichiometric changes of m¹A at position 58 of the tRNA-Glu after AlkB-treatment. The percentage of m¹A is reduced from 73% to 0% at the position where it is demethylated completely and becomes 100% A. (FIG. 5D) I. Structural change of m¹A to A at position 58 caused by AlkB treatment. II-III. The disappearance of the branch of the methylated ladder in 2D/R-mass plot caused by AlkB treatment. The 2-O′-methyl of m⁵Um at position 54 of the tRNA-Glu blocks the acid degradation, causing a small mass gap with a ladder fragment of 640.0819 Da (the mass sum of likely m⁵Um and U) missing between positions 54 and 55.

FIG. 6A-H. MLC-Seq analysis of yeast total tRNA and modifications. (FIG. 6A) Results of LC-MS analysis for intact tRNA molecules; orange points represent a match found between the data and a published tRNA sequence. Out of the 129 masses, only 64 match the calculated tRNA masses from the tRNAdb 2009 database and are highlighted in orange (Table 2). (FIG. 6B) Homology search results indicating partial 3′-end truncations; molecules with mass differences of 329.05 or 305.04 Da indicate that their structures differ by an A or C nucleotide, respectively. (FIG. 6C) Homology search results indicating partial methylations; molecules with mass differences of 14 Da indicate that their structures may differ by a methyl group. (FIG. 6D) Percentages of tRNA molecules across all subtypes possessing-CCA, -CC, and -C 3′ ends. (FIG. 6E) Structures and mass data for amino acid-charged tRNA molecules, indicating that MS analysis can effectively identify complete tRNA molecules charged with amino acids. (FIG. 6F) Partial sequences can be read out by ladder fragments computationally separated by the MassSum algorithm, utilizing the intact masses of a specific tRNA-AspGUC isoform (23904.22 Da). (FIG. 6G) Using BLAST to search for the partial sequences and related positions obtained from FIG. 6F (shown in boxes) returns a positive result, validating the data and indicating the tRNA subtype that these fragments belong to. (FIG. 6H) Table of nucleotides confirmed through cross-referencing between LC-MS data and reference sequences, and a list of symbols used by BLAST and their corresponding nucleotide modifications. (SEQ ID NO. 7-38; SEQ ID NO. 40) Shaded cells indicate nucleotides that were confirmed by 5′ and/or 3′ ladder fragments; cells with borders indicate nucleotide modifications that were confirmed by 5′ and/or 3′ ladder fragments. Colors in positions 74-76 indicate the relative amounts of each tRNA subtype with -CCA, -CC, and -C 3′ ends and in other positions indicate the stoichiometry of each modification at the site. For better visualization, the stoichiometry of partial RNA modifications and 3′ terminal truncations was represented using a six-color scale at intervals of 0, 20, 40, 60, 80, and 100. Quantification was limited to partial 3′-end truncations and partial modifications that were confirmed by at least two newly branched ladder fragments after the modification site. Additional details can be found in FIGS. 12-19 and Tables 2-4.

FIG. 7A-B. Detailed illustration for data analysis procedure in FIG. 1. (FIG. 7A) Homology Search before acid degradation. From the intact RNA mass level, the RNA sequence diversity and partial modification information are preserved, showing the number of RNA species in a given sample and if and how they related to a specific tRNA. I-II, Homology search allows related tRNAs to be identified. After acid degradation, in order to sequence tRNA, (FIG. 7B) a complete set of stepwise innovative computational tools/algorithms was developed to synergize MS information at both ladder fragment level and intact RNA level, including: I-II, Homology search, in this step, isoform 1 with masses of 24610.4911 Da before acid degradation, for example, shifted to 24252.3692 Da after acid degradation with a mass difference of 358.1219 Da, indicating that there are acid-labile nucleotide modifications. III, For each isoform identified, MassSum algorithm was applied to isolate all its ladder fragments (pairing one in 3′-ladder and the other in 5′-ladder) from the complex MS data of the RNA sample. IV, Results using GapFill to find non-paired ladder fragments that are missed by MassSum. After ladder separation, 3′ and 5′-ladder (V) are obtained separately. VI, Ladder Complementation to perfect MS ladders, allowing direct sequencing of full-length tRNAs. This figure is for illustrative purposes only.

FIG. 8A-B: Detection of acid-labile nucleotides. (FIG. 8A) Masses of the five intact tRNA-Phe isoforms before and after acid degradation; the mass loss of approximately 358.2 Da indicates the presence of an acid labile nucleotide modification, which was further identified as a wybutosine (Y). (FIG. 8B) Structure of Y and its depurinated ribose form Y′ which forms under acidic conditions.

FIG. 9A-B Supplementary MLC-Seq results of yeast tRNA-Phe. (FIG. 9A) Observed branching indicates the truncated 3′-end isoforms and the partial editing/transition position and stoichiometry. The theoretical exact mass and observed monoisotopic mass after acid degradation are presented, along with the relative abundance before acid degradation. (FIG. 9B) Mean ratio and standard deviation for partial transition at position 67 of the tRNA-Phe.

FIG. 10: tRNA-Glu PAGE gel image (left) and Northern blot image (right) confirming 125 enriched tRNA-Glu from mouse liver.

FIG. 11A-C. Using isotopic distributions to calculate stoichiometries in partial modifications with small mass differences. (FIG. 11A) Isotopic distributions for position 16 of tRNA-Gln, which contains a partial U-D dihydrogenation. Red: distribution from LC-MS data; blue: computed distributions for U only; green: computed distribution for a composite distribution of 32.8% U 135 and 67.2% D. (FIG. 11B) Breakdown of the composite U-D distribution, showing the contributions from the U (light green) and D (dark green) branches for each m/z value. (FIG. 11C) Cumulative probability functions for isotopic distributions obtained from LC-MS data (red), calculations for U only (blue), and the composite U-D distribution (green, dashed to allow view of both green and red lines). The blue dotted line shows the KS statistic for the calculated U distribution, i.e., the maximum distance between the cumulative probability functions of the LC-MS data and the 141 calculation distribution for U only.

FIG. 12A-B. LC-MS chromatographic analyses of yeast total tRNA. The analysis was conducted via BioPharma Finder 5.0 software (ThermoScientific) using the Xtract (isotopically resolved) algorithm. (FIG. 12A) Total ion chromatogram (TIC) showcasing the comprehensive ion spectrum of a yeast total RNA sample. (FIG. 12B) Stacked extracted ion chromatograms (EICs) delineating each detected yeast tRNA type individually.

FIG. 13A-D. MS intensity analysis of yeast total tRNA ladder fragments via MLC-Seq. (FIG. 13A) This panel shows nucleotides or modifications confirmed by 5′ ladder fragments originated from full-length tRNAs. (FIG. 13B) This panel presents nucleotides or modifications verified by 3′ ladder fragments originated from full-length tRNAs. (FIG. 13C) Confirmed nucleotides or modifications by 3′ ladder fragments originated from tRNAs lacking a terminal “A” at the 3′ end. (FIG. 13D) Verified nucleotides or modifications by 3′ ladder fragments originated from tRNAs missing a terminal “CA” at the 3′end. The figure categorizes ladder fragments into 5′ (Panel a) and 3′ (Panels b/c/d) to elaborate on FIG. 6H. Each cell in the panels represents nucleotides or modifications confirmed by either 5′ or 3′ ladder fragments. Ten distinct colors denote various intensity levels, with each color corresponding to a confirmed nucleotide or modification. Colored cells indicate confirmed nucleotides; boxed cells signify 161 verified modifications. For precise nucleotide positions, refer to Table 3.

FIG. 14A-H. MLC-Seq analysis of individual tRNA-AspGUC within a yeast total tRNA sample. (FIG. 14A) Cloverleaf structure of tRNA-AspGUC from the tRNAdb 2009 database, utilized to compute the intact masses of tRNA-AspGUC and its related isoforms. (FIG. 14B) Detected intact masses of tRNA-AspGUC and its various isoforms-including truncated-CC (lacking a terminal nucleotide “A” at the 3′ end), truncated-C (lacking two terminal nucleotides “CA” at the 3′ end). (FIG. 14C) Table showcasing results of paired ladder fragments computationally separated by the MassSum algorithm using the intact mass of tRNA-AspGUC (24233.28 Da). The mass sum of any pair of the ladder fragments (comprising one 5′- and one 3′-ladder fragments) generated by a cleavage event during acid hydrolysis is a constant, equal to the intact mass of tRNA-AspGUC plus the mass of a water molecule. (FIG. 14D) Ladder fragments sorted by the MassSum algorithm using tRNA-AspGUC truncated-CC isoform's intact mass (23904.22 Da) exhibit a sigmoidal trend in the tR-mass plot. Each fragment is part of either the 5′ or 3′ ladder, collectively constituting the tRNA-AspGUC sequence ladders. (FIG. 14E) The plot displays two distinct tR-mass ladders with identical sequences beginning at position 56 of the RNA-AspGUC. These ladders represent a segment of the tRNA-AspGUC 3′-CC sequence. A mass difference of approximately 21.98 Da between corresponding ladder fragments in each ladder explicitly indicates sodium attachment, highlighted in red. (FIG. 14F) The EIC of the intact tRNA-AspGUC truncated C and CC isoforms. (FIG. 14G) An overall 96.0% sequence coverage was achieved for tRNA-AspGUC after integration of cross referencing and mass ladder complementation. (FIG. 14H) List of site-specific modifications. Base calling of nucleotide modifications is primarily determined by mass differences of successive 5′ ladder fragments originated from tRNA-AspGUC (75 nt, full-length), and is subsequently confirmed by 3′ ladder fragments of the full-length tRNA-AspGUC and 3′ ladder fragments of its truncated isoforms (3′-CC and 3′-C). Depth information indicates the number of times each nucleotide was read independently from the tRNA isoform ladders. The stoichiometry of mU at the 54th position stands at a precise 98%.

FIG. 15A-F. MLC-Seq analysis of individual tRNA-ValIAC within a yeast total tRNA sample. (FIG. 15A) The cloverleaf structure of tRNA-ValIAC, sourced from the tRNAdb 2009 database, was used for calculating the intact masses of tRNA-ValIAC and its isoforms. (SEQ ID NO. 39) (FIG. 15B) An overall 98.7% sequence coverage was achieved for tRNA-ValIAC after integration of cross-referencing and mass ladder complementation. (FIG. 15C) The table displays the observed intact masses for all three tRNA-ValIAC isoforms, along with one methylated form of truncated-CC. Each matches its theoretical mass within a 10 ppm difference. (FIG. 15D) The EIC of the intact tRNA-ValIAC C isoform, missing two nucleotide ‘CA’ at the 3′ end. (FIG. 15E) Acid-hydrolyzed tRNA-ValIAC (cross-referenced) ladder fragments are shown in distinct sigmoidal curves. Each fragment corresponds to a sequencing ladder rung, as measured by LC-MS. Mass differences between the rungs reveal the specific RNA nucleotide at that position, whether modified or not. (FIG. 15F) List of site-specific modifications. Base-calling of nucleotide modifications is primarily determined by mass differences of successive 5′ ladder fragments originated from the full-length tRNA-ValIAC, and is subsequently confirmed by 3′ ladder fragments of the full-length tRNA-ValIAC and 3′ ladder fragments of its truncated isoforms (3′-CC and 3′-C).

FIG. 16A-D. MLC-Seq analysis of individual tRNA-IleIAU within a yeast total RNA sample. (FIG. 16A) The cloverleaf structure of tRNA-IleIAU, sourced from the tRNAdb 2009 database, was used for calculating the intact masses of tRNA-IleIAU and its isoforms. (FIG. 16B) Attained an overall 96.1% sequence coverage for tRNA-IleIAU after integration of cross-referencing and mass ladder complementation. (FIG. 16C) The EIC of the intact tRNA-IleIAU CC isoform, missing an ‘A’ at the 3′ end. (FIG. 16D) List of site-specific modifications. Base-calling of nucleotide modifications is primarily determined by mass differences of successive 5′ ladder fragments originated from tRNA-IleIAU (full-length 3′ CCA), and is subsequently confirmed by 3′ ladder fragments of the full-length tRNA-IleIAU and 3′ ladder fragments of its truncated isoforms (3′-CC and 3′-C). Depth information indicates the number of times each nucleotide was read independently from the tRNA isoform ladders. Stoichiometry of mG at the 9th position is 92%.

FIG. 17A-D. MLC-Seq analysis of individual tRNA-LysCUU within a yeast total tRNA sample. (FIG. 17A) The cloverleaf structure of tRNA-LysCUU, sourced from the tRNAdb 2009 database, was used for calculating the intact masses of tRNA-LysCUU and its isoforms. (FIG. 17B) An overall 96.1% sequence coverage was achieved for tRNA-LysCUU after integration of cross referencing and mass ladder complementation. (FIG. 17C) The table details the observed intact masses for two isoforms of tRNA-LysCUU, specifically the truncated-CC form (lacking a 3′ terminal “A”) and the truncated-C form (missing two 3′ terminal nucleotides “CA”). Each observed mass is in close agreement with its theoretical value, with a ppm error under 10. Relative abundances for these isoform variations are also provided. (FIG. 17D) List of site-specific modifications. Base calling of nucleotide modifications is primarily determined by mass differences of successive 5′ ladder fragments originated from the full-length tRNA-LysCUU and is subsequently confirmed by 3′ ladder fragments of the full-length tRNA-LysCUU and 3′ ladder fragments of its truncated isoforms (3′-CC and 3′-C). Depth information indicates the number of times each nucleotide was read independently from the tRNA isoform ladders. Stoichiometry of mG at the 9th position is 72±16%.

FIG. 18A-D. MLC-Seq analysis of individual tRNA-Arg1CG within a yeast total tRNA sample. (FIG. 18A) The cloverleaf structure of tRNA-ArgICG, sourced from the tRNAdb 2009 database, was used for calculating the intact masses of tRNA-Arg1CG and its isoforms. (FIG. 18B) An overall 94.7% sequence coverage was achieved for tRNA-ArgICG after integration of cross-referencing and mass ladder complementation. (FIG. 18C) The table displays the observed intact masses and relative abundances of tRNA-ArgICG isoform variations, including truncated-CC and methylated forms. Each observed mass closely aligns with its theoretical counterpart, with a ppm error under 10. (FIG. 18D) List of site-specific modifications. Base-calling of nucleotide modifications is primarily determined by mass differences of successive 5′ ladder fragments originated from the full-length tRNA-ArgICG, and is subsequently confirmed by 3′ ladder fragments of the full-length tRNA ArgICG and 3′ ladder fragments of its truncated isoforms (3′-CC and 3′-C). Depth information indicates the number of times each nucleotide was read independently from the tRNA isoform ladders. The stoichiometry of mA at the 58th position is 47%.

FIG. 19A-E. MLC-Seq analysis of individual tRNA-AsnGUU within a yeast total tRNA sample. (FIG. 19A) The cloverleaf structure of tRNA-AsnGUU, sourced from the tRNAdb 2009 database, was utilized to calculate the intact masses of tRNA-AsnGUU and its related isoforms. (FIG. 19B) An overall sequence coverage of 93.5% is achieved for RNA-AsnGUU after integration of cross-referencing and mass ladder complementation. (FIG. 19C) This table presents the observed intact masses of tRNA-AsnGUU and its variant isoforms, detailing relative abundance. The variations include full-length tRNA-AsnGUU, truncated-CC (missing a “A” at the 3′ end), and their methylated isoforms. Each isoform's mass aligns with the theoretical mass, maintaining a difference within 10 ppm. (FIG. 19D) The EIC of the intact tRNA-AsnGUU (25051.48 Da). (FIG. 19E) List of site-specific modifications. Base-calling of nucleotide modifications is primarily determined by mass differences of successive 5′ ladder fragments originated from the full-length tRNA AsnGUU and is subsequently confirmed by 3′ ladder fragments of the full-length tRNA AsnGUU and 3′ ladder fragments of its truncated isoforms (3′-CC and 3′-C). Depth information indicates the number of times each nucleotide was read independently from the tRNA isoform ladders. At the 58th position, the stoichiometry of mA is 18±6%.

### DETAILED DESCRIPTION

Although the present disclosure will be described in terms of specific embodiments, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

The current disclosure is related to direct, liquid-chromatography-mass spectrometry-based RNA sequencing methods, referred to herein as MLC-Seq, which can be used to directly sequence RNA without cDNA synthesis, simultaneously determine the nucleotide sequence of RNA molecules with single nucleotide resolution as well as detection of the presence of any nucleotide modifications that an RNA molecule carries. Specifically, the disclosed methods can be used to determine the type, location and quantity of nucleotide modifications within the RNA sample. The RNA to be sequenced may be a purified RNA sample of limited diversity, as well as samples of RNA containing complex mixtures of RNA, such as RNA derived from a biological sample. Such techniques can be used to determine the nucleotide (modified or canonical) sequence of an RNA molecule and to advantageously correlate the biological functions of any given RNA molecule with its associated modifications.

As used herein, ribonucleic acid (RNA) refers to oligoribonucleotides or polyribonucleotides as well as any analogs of RNA, for example, made from nucleotide analogs. The RNA will typically have a base moiety of adenine (A), guanine (G), cytosine (C) and uracil (U), a sugar moiety of a ribose and a phosphate moiety of phosphate bonds. RNA molecules include both natural RNA and artificial RNA analogs. The RNA can be synthetic or can be isolated from a particular biological sample using any number of procedures which are well known in the art, wherein the particular chosen procedure is appropriate for the particular biological sample. RNA samples include for example, coding RNA and non-coding RNA such as mRNA, rRNA, tRNA, antisense-RNA, and siRNA, to name a few. No limitations are imposed on the base length of RNA. The MLC-Seq sequencing methods disclosed herein enable the sequencing of not only purified RNA samples, but also more complicated RNA samples containing mixtures of different RNAs.

In a specific embodiment, the structure of synthetic oligoribonucleotides of therapeutic value can be determined using the sequencing methods disclosed herein. Such methods will be of special valuable to those engaged in research, manufacture, and quality control of RNA-based therapeutics, as well as the regulatory entities. Incorporation of structural modifications into synthetic oligoribonucleotides has been a proven strategy for improving the polymer's physical properties and pharmacokinetic parameters. However, the characterization and the structure elucidation of synthetic and highly modified oligonucleotides remains a significant hurdle.

In one aspect, the present disclosure provides a method for generating the sequence of one or more RNA molecules and detecting the presence, identity, location, and quantity of RNA nucleotide modifications on said one or more RNA molecules, said method RNA comprising as an initial step (i) separating an RNA sample into two samples, one intact and one fragmented to form sequencable ladder fragments such as 5′ and 3′ MS ladder fragments; (ii) mass measurement of resultant RNA fragments and mass measurement of the intact RNA; and (iii) data processing steps, including identification and separation of 3′ and/or 5′ MS ladder fragments thereby generating the sequence of one or more RNA molecules and detecting the presence, identity, location, and quantity of RNA nucleotide modifications. The provided MLC-SEQ sequencing method advantageously addresses incomplete ladder issues associated with MS ladder sequencing of RNAs by providing a number of processing steps that can be used to address and resolve said issues.

As one step in the sequence methods disclosed herein, the RNA to be sequenced is subjected to well-controlled acid hydrolysis degradation. As used herein, the terms degradation and cleavage may be used interchangeably. It is understood that the degradation, or cleavage, of RNA refers to breaks in the RNA strand resulting in fragmentation of the RNA into two or more fragments. In general, such fragmentation for purposes of the present disclosure are random along any of RNA phosphodiester bonds. By controlling the timing of exposure to a degradation reagent, single but randomized cleavage along the target RNA molecule backbone may be achieved, thus simplifying downstream MS data analysis.

In an embodiment, chemical cleavage is accomplished through use of formic acid. Formic acid degradation is preferred because its boiling point is approximately 100° C. like water and the formic acid can be easily remove it e.g., by lyophilizer or speedvac. Such cleavage is designed to cleave the RNA molecule at its 5′-ribose positions throughout the molecule. In addition to formic acid degradation, alkaline degradation may also be used. For example, the following alkaline buffers may be used to degrade the RNA sample: 1× Alkaline Hydrolysis Buffer (e.g., 50 mM Sodium Carbonate [NaHCO₃/Na₂CO₃] pH 9.2, 1 mM EDTA; or the Alkaline Hydrolysis Buffer supplied with Ambion's RNA Grade Ribonucleases), or Ammonia aqueous solution (NH₄OH, 25%-32%). In addition to chemical cleavage, RNAs may be subjected to enzymatic degradation. Enzymes that may be used to degrade the RNA include for example, Crotalus phosphodiesterase I, bovine spleen phosphodiesterse II and XRN-1 exoribonucease. Such RNA degradation treatment is carried out under conditions where a desired single cleavage event occurs on the RNA molecule resulting in a pool of differently sized RNA fragments resulting in a complete ladder. Similarly, DNA can also be enzymatically degraded into ladder fragments, which can be sequenced using the MS-based sequencing.

Once RNA fragment pools are formed, the RNA fragments, as well as intact RNA, can be analyzed by any of a variety of means including liquid chromatography coupled with mass spectrometry, or gas chromatography coupled with mass spectrometry, or ion-mobility spectrometry coupled with mass spectrometry, or capillary electrophoresis coupled with mass spectrometry, or other methods known in the art. Preferred mass spectrometer formats include continuous or electrospray ionization (ESI) and related methods or other mass spectrometer that can detect RNA fragments like MALDI-MS. HPLC-MS measurements can be performed using high resolution time-of-flight or Orbitrap mass spectrometers that have a mass accuracy of less than 5 ppm.

LC-MS data is then converted into RNA ladder sequence information. The unique mass tag of each canonical ribonucleotide and its associated modifications on the RNA molecule, allows one to not only determine the primary nucleotide sequence of the RNA but also to determine the presence, type and location of RNA modifications. When an RNA is not 100%, each of the RNA ladder fragments carries stoichiometry information, which allows stoichiometric quantification of each nucleotide modification site-specifically.

Mass adducts can be removed from the deconvoluted data and the sequences will be predicted/generated using both mass and retention time data. The retention time-coupled mass data for the fragments is analyzed to determine which data points are “valid” and to be used for subsequent sequence determination and which data points are to be filtered out. After data reduction step, the mass difference (m) between two adjacent RNA fragments [m=m(i)−m(i−1), 1<i<n, n=RNA length], where m(i) is the mass of any ladder fragment and m(i−1) is the preceding lower mass ladder fragment, and match such mass differences with the exact masses of known nucleotide fragments to correlate the derived RNA sequencing information based on mass differences to determine the RNA sequence and its modification. As long as the structural modification on an RNA nucleoside is mass-altering, the disclosed sequencing method will permit identification of the RNA sequence and its modification to be identified. The mass of all the known modified ribonucleosides can be conveniently retrieved from known RNA modification databases (12).

In another embodiment, an RNA sequencing technique is provided that enhances the read length and throughput, allowing direct and simultaneous sequencing of not only predominantly major RNA but also at the same time even low stoichiometric RNA, such as tRNA, tsRNA, tRNA isoforms/species directly from a complex sample without intensive sample preparation and in the presence of imperfect ladder formation. The method includes the use of novel computational methods and tools for determining the sequence and presence of modified bases in mixtures of RNA, including those of tRNA samples.

Accordingly, the present disclosure provides a MLC-Seq platform that employs three functionalities, including: (1) de novo sequencing (without sequence input) to identify tRNA types present in the samples, (2) site-specific mapping of tRNA modifications by cross referencing between tRNA database sequences and the LC-MS data⁴³ and (3) site-specific quantification of partial tRNA modification stoichiometry.

More specifically, the present disclosure provides a method for de novo sequencing of tRNAs and site-specific quantification of RNA modification stoichiometries wherein said method comprises as a first step starting with a tRNA sample for sequencing that is divided into two samples. One half of the sample is referred to as intact (no acid hydrolysis) while the other half of the sample is subjected to controlled acid hydrolysis. The method further comprises the step of direct observation of partial nucleotide modifications or editing in the intact RNA sample. The method further comprises the step of conducting MS ladder sequencing of the RNA sample subjected to controlled acid hydrolysis. In the RNA sample subjected to controlled acid hydrolysis the RNA is converted into two series of ladders (5′ and 3′) composed of a series of fragments of varying lengths for MS ladder sequencing. After acid degradation, the 5′ and 3′ ladders display sigmoidal curves on a tR-mass plot, where branches in the plot indicate the position and types of partially modified or edited nucleotides. In this step, de novo base calling of the complete sequence of the tRNA isoforms, may be accomplished using novel algorithms (data processing) that identify and separate each tRNA species or isoform's MS ladders from LC-MS data. As another step in the method, site-specific quantification of stoichiometry for partial tRNA nucleotide modifications and editing is accomplished using data from both intact and ladder levels. EIC peak areas of each fragment indicates the stoichiometry ratio, e.g., modification of the tRNA at a given position. Ladder level quantification aligns with relative abundances at the intact level, confirming initially observed modifications or editing.

Details of the sequencing method are described below for tRNA molecules, but it is to be understood that said method can be applied equally as well to any RNA.

The method provided herein includes as a first step, separating an RNA sample into two samples, one is intact and the other is fragmented through controlled RNA degradation by exposure to, for example, acid hydrolysis. In a specific embodiment of the present disclosure, formic acid, may be applied to degrade tRNA samples for producing mass ladders, according to reported experimental protocols. In a non-limiting embodiment, the tRNA sample solution may be divided into three equal aliquots for formic acid degradation using 50% (v/v) formic acid at 40° C., with one reaction running for 2 min, one for 5 min and one for 15 min. for controlled exposure of the RNA to different levels of acid hydrolysis. Ideally, the goal of the degradation step is a single cleavage of each RNA molecule resulting in a ladder of 5′- and 3-ladders that are subsequently measured thorough an LC-MS step.

In another step, the acid-hydrolyzed tRNA samples as well as the intact sample, are separated and analyzed through LC-MS measurements well known to those of skill in the art. In an embodiment, on a Orbitrap Exploris 240 mass spectrometer coupled to a reversed-phase ion-pair liquid chromatography (ThermoFisher Scientific, USA) can be used using 200 mM HFIP and 10 mM DIPEA as eluent A, and methanol, 7.5 mM HFIP, and 3.75 mM DIPEA as eluent B. A gradient of 2% to 38% B in 15 minutes was used to elute RNA samples across a 2.1×50 mm DNAPac reversed-phase column. The flow rate was 0.4 mL/min, and all separates were performed with the column temperature maintained at 40° C. Injection volumes were 5-25 μL, and sample amounts were 20-200 pmol of tRNA. tRNAs were analyzed in a negative ion full MS mode from 410 m/z to 3200 m/z with a scan rate of 2 spectrum/s at 120 k resolution. The sample data is processed using the Thermo BioPharma Finder 4.0 (ThermoFisher Scientific, USA), and a workflow of compound detection with deconvolution algorithm is used to extract relevant spectral and chromatographic information from the LC-MS experiments as described previously. In the disclosed method for de novo base calling of the complete sequence of the tRNA isoforms, novel algorithms (data processing) may be used that identify and separate each tRNA species or isoform's MS ladders from LC-MS data. In one embodiment, as a data processing step, a homology search can be performed. The computer-implemented method comprises a step for identifying tRNA isoforms based on a homology search function configured to divide the intact RNA molecules into two or more groups with each group representing one specific RNA species and its related isoforms. In such an embodiment, the homology search can be performed before or after degradation of the RNA.

More specifically, once LC-MS data are displayed as a two-dimensional (2D) t_{R}-mass plot, a homology search of intact tRNAs can be conducted in the monoisotopic mass range of >˜24 k Da using an in-house algorithm in Python (see GitHub). This algorithm identifies related tRNA isoforms that may share the same ancestral precursor tRNA but are different in absolute sequence, e.g., in posttranscriptional profiles of nucleotide modifications, editing, and truncations. Mass differences between two intact tRNA isoforms are calculated and matched to the known mass of nucleotides or nucleotide modifications in the database.²⁶ For example, a difference of 14.0157 Da (±10 ppm)⁴⁵ can be assigned to a methylation (Me/-CH2—) event, while a difference of 329.0525 Da corresponds to an additional A nucleotide. Therefore, these intact tRNAs are assigned to the same tRNA group and considered homologous isoforms of a specific tRNA for sequencing together. Said homology search serves as a nontarget preselection step to group possible related tRNA isoforms together for sequencing.

In another embodiment, a data processing step referred to as MassSum Data Separation may be used. MassSum is an algorithm in Python (see Github) developed based upon the controlled acid hydrolysis of RNA. MassSum takes advantage of the fact that the sum of the masses of each pair of fragments (5′ and 3′) produced from a single cut of an intact RNA is a constant value unique for each RNA isoform/species

$$\text{?} + \text{?} = \text{?} + \text{?} \qquad ( 1 )$$

$$\text{?} \text{indicates text missing or illegible when filed}$$

where mass_{intact} is the intact RNA, mass_{3′} and mass_{5′} are the two fragment masses, and mass_{H2}O is the mass of one water molecule. This equation can be employed to isolate ladder compounds corresponding to a specific isoform, which simplifies the data set by grouping MS ladder components into subsets, one for each tRNA isoform/species. MassSum operates by choosing two random compounds from the acid-degraded LC-MS data set and adding their masses; if the sum is equal to the mass of a known isoform/species, the fragments are selected into a subset corresponding to that isoform/species containing all its 3′ and 5′ ladders.

In yet another embodiment, a GapFill data processing step may be employed. GapFill is another Python-based algorithm (see GitHub) developed to complement MassSum, which identifies pairs of corresponding 5′/3′ fragments but cannot separate data if, e.g., there is no 5′ fragment found in the data that pair with a given 3′ fragment. In one aspect, the GapFill data processing step may be used to “rescue” any ladder fragments missed by MassSum separation by first identifying gaps where fragments are missing from a ladder after the MassSum algorithm is applied and the corresponding values of mass_{low} and mass_{high}, the masses of, respectively, the heaviest known fragment below the gap range and the lightest known fragment above the gap range. The data set typically contains several fragments whose mass falls between mass_{low} and mass_{high}, but presumably were not selected by the MassSum algorithm during data separation. GapFill iterates over each fragment LC-MS data set whose mass falls within this range and examines the mass differences between this compound and mass_{low} and mass_{high}. If the mass difference is equal to the sum of one or more nucleotides or modifications in the RNA modification database,²⁶ it is noted as a connection. If the fragment in the gap has connections with both ending fragments, it is selected into a candidate pool for the subsequent sequencing process. After iteration, GapFill calculates connections of the fragments in the candidate pool and the frequency of each connection, and the fragments with the highest frequency are chosen to fill in the gap.

In yet another embodiment, a Ladder Complementation data processing step may be utilized. For example, after MassSum and GapFill, each tRNA isoform has its own set of separate 5′ and 3′ ladders. If any ladder is perfect (i.e., without any missing fragments), the full RNA sequence can be read, from the first to the last nucleotide in the sequence. Incomplete ladders can be completed using other related isoforms to obtain a more complete ladder for sequencing. A Python-based computational algorithm (see Github) was designed to align ladders from related isoforms based on the position of the ladder fragment in the 5′/3′ direction and may be used. For example, the 5′ ladders for the RNA, are positioned horizontally so that the nucleotide positions are aligned. Ladder complementation is then performed separately on 5′ or 3′ ladders (but not mixed ladders), resulting in one final 5′ ladder or one final 3′ ladder. Additionally, the 3′ fragments can be converted to their corresponding 5′ fragments for each tRNA isoform based on the MassSum processing. As such, each position in an tRNA isoform could have its original 5′ ladder fragment as well as a second fragment converted from the corresponding 3′ fragment, which can be used for confirmation and/or complementation.

The present disclosure advantageously provides a novel method for de novo sequencing of tRNAs that permits site-specific quantification of RNA modification stoichiometries. The stoichiometries of partial nucleotide modifications/alterations are quantified based on integrating EIC peaks corresponding to two or more fragments present at a single position in a sequence. EIC chromatograms may be generated via BioPharma Finder 5.0-5.2 software (Thermo Scientific) using the Xtract (isotopically resolved) algorithm. In general, each EIC trace uses a single m/z value corresponding to a fragment's most abundant isotope and the charge state z with the strongest MS signal; in cases where fragments at a single position have different preferred charge states, the preferred charge state for the more abundant fragment (i.e., with the greatest EIC area among all fragments of interest) is used. The ratio of EIC areas is taken as the relative abundance of their respective fragments. Each modification creates a branch in the MS ladder that is evident in all subsequent positions in the sequence, so this calculation is repeated in multiple positions for each partial modification to obtain multiple values that are used to calculate averages and standard deviations. In one aspect, where partial components at a position are close in mass to each other (mass difference of 2 Da or less), the isotopic patterns of the fragments overlapped significantly, such that the most abundant m/z values feature contributions from both fragments. In such an instance, a composite isotopic pattern is calculated and compared to the pattern obtained from MS data.

In one aspect, stoichiometries may be solved using a search to determine, to the nearest tenth of a percent, the composition whose theoretical composite isotopic pattern best matched the data pattern based on minimizing the Kolmogorov-Smirnov (KS) statistic between the two isotopic distributions; this statistic is used because its value is not dependent on the test sample size, making it easier to apply to MS data where the number of “observations” is ambiguous.

In an embodiment of the invention, the disclosed MLC-Seq method may be used to track changes in RNA modifications. These changes can be caused by diseases or cellular disturbances whose progression can be tracked through monitoring the identity and stoichiometry changes in RNA nucleotides (modified or canonical). To examine this, the RNA sample may be treated with AlkB to leverage its selectivity toward specific isomeric methylated nucleotides.^{39,40} AlkB reacts with m¹A, m¹G, and m³C (converting them to their respective canonical nucleotides) but is inert toward m⁶A, m²G, and m⁵C. Measuring site-specific changes in modification stoichiometry can verify AlkB's reactivity toward specific methylated nucleotides while demonstrating the capacity of MLC-Seq to quantitatively track changes in tRNA samples site-specifically at the single-nucleotide level.

In another aspect, acid-labile nucleotides may be identified using an algorithm in Python (see GitHub) that analyzes the connections between the compounds (with a monoisotopic mass >24 kDa for tRNAs) measured by LC-MS before and after acid degradation. For each such compound pair, if the monoisotopic mass difference can be matched to a known mass difference corresponding to a possible structural change to a nucleotide modification during acid hydrolysis (or the sum of several such changes), the compound pair will be selected and further considered to potentially contain acid-labile nucleotide modifications. In general, if the intact mass of the RNA species does not change after acid degradation, this intact mass will be used for MassSum data separation. Otherwise, the presence of acid-labile nucleotides may be indicated by matching the observed mass difference with the theoretical mass difference caused by an acid-mediated structural change in a nucleotide or a combination of several such changes.

In another embodiment, a method for site-specific identification of single nucleotide substitutions and/or partial RNA nucleotide modifications in an RNA molecule, in a sample comprising a mixture of RNAs and wherein the length of the RNA molecule is more than 20 nucleotides wherein the method comprises (i) receiving liquid chromatography-mass-spectrometry (LC-MS) data of an RNA sample, where the RNA sample contains modified nucleoside pseudo-U labeled with CMC resulting in a mass and retention time branch shifting from the non-CMC-converted mass-retention time curve, and wherein said RNA is subjected to controlled acid hydrolysis after CMC labeling, analyzing the LC-MS data of the labeled RNA; (ii) filtering the LC-MS data based on mass, the filtering including removing masses smaller than a predetermined size; and analyzing the filtered LC-MS data, to determine if there is one or more ladder branch in the two dimension mass-retention time plot. Said analysis of the filtered LC-MS data includes (a) determining a mass difference between at least two adjacent ladder fragments; (b) determining whether the mass difference is equal to at least one of a canonical nucleotide, or a modified nucleotide; and (c) reading-out more than one RNA sequence in parallel, with one containing non-modified RNA canonical nucleotide, the other(s) containing modified or substituted counterpart, or one containing one modified RNA canonical nucleotide, the other(s) containing differently modified or substituted counterpart, as a sequence read after determining no remaining valid nucleotides in the remaining LC-MS data, the RNA sequence including a sequence order of each identified canonical nucleotide and any identified modified nucleotides.

The present disclosure provides a kit for use in generating the sequence of one or more RNA molecules and detecting the presence, identity, location, and quantity of RNA nucleotide modifications on said one or more RNA molecules, said kit comprising one or more components for performance of the de novo sequencing method disclosed herein. Said kit may comprise components for the (i) controlled fragmentation of the RNA to form sequencable ladder fragments such as 5′ and 3′ MS ladder fragments; (ii) mass measurement of resultant degraded RNA samples containing RNAs and their fragmented fragments; (iii) data processing, including identification and separation of 3′ and/or 5′ MS ladder fragments thereby generating the sequence of one or more RNA molecules and detecting the presence, identity, location, and quantity of RNA nucleotide modifications and/or (iv) site-specific quantification of RNA modification stoichiometries based on integrating EIC peaks.

The present disclosure provides a computer-implemented method for determining an order of nucleotides and/or modifications of an RNA molecule, wherein the method includes: receiving/exporting liquid chromatography-mass-spectrometry (LC-MS) data of an RNA sample, the LC-MS data including but not limited to a mass (e.g., m/z, monoisotopic mass, average mass), charge states, retention time (RT), Height, width, volume, relative abundance, and quality score (QS); filtering the LC-MS data based on mass, the filtering including removing masses smaller than a predetermined size; analyzing the filtered LC-MS data, to determine a plurality of RNA sequences, analyzing the filtered LC-MS data including: determining a mass difference between at least two adjacent ladder fragments; and determining whether the mass difference is equal to at least one of a canonical nucleotide, or a modified nucleotide (known or unknown); and reading-out an RNA sequence as a sequence read after determining no remaining valid nucleotides in the remaining LC-MS data, the RNA sequence including a sequence order of each identified canonical nucleotide and any identified modified nucleotides.

In an embodiment of the invention, a computer-implemented sequencing method is provided for determining the Mass Sum of any of two ladder fragments; and if the mass sum is equal to the mass of the intact RNA (detected in homology search) plus the mass of a water, isolating these two fragments into a pair based on the determined MassSum for sequencing of the RNA molecule. In an embodiment, MassSum may not be related to any two adjacent ladder fragments. Further, MassSum may not be limited to computational separate ladder fragments generated by one cleave per RNA molecule but may also be used to separate other fragments of RNA that gets cleaved more than once.

In another embodiment, a computer-implemented method is provided comprising the step of determining if any of the two ladder fragments cannot pair based on the mass sum value for a given RNA, and if so finding one of them by use of a GapFill algorithm, configured to search for ladder fragments missed by MassSum determination. In another embodiment, the computer-implemented method comprises the step of determining presence, type, location, or quantity of the modified nucleotides within the RNA molecule. In an embodiment, a computer-implemented method is provided comprising the step of separating the 5′- and 3′end fragments of each identified tRNA isoform based on breaking two adjacent sigmoidal curves into two isolated curves.

In an embodiment, provided is a non-transitory computer-readable medium storing instructions that, when executed by a processor, cause the processor to perform a method for generating the sequence of one or more RNA molecules and detecting the presence, identity, location, and quantity of RNA nucleotide modifications on said one or more RNA molecules, the method comprising the steps of (i) identifying a specific chemical moiety associated with the RNA (ii) controlled fragmentation of the RNA to form 5′ and 3′ MS ladder fragments; (iii) mass measurement of resultant degraded RNA samples containing RNAs and their degraded fragments; and (iv) data processing, including identification of 3′ and/or 5′ MS ladder fragments thereby generating the sequence of one or more RNA molecules and detecting the presence, identity, location, and quantity of RNA nucleotide modifications.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Python, Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions

### Example 1

### Materials and Methods

### Reagents and Chemicals

All chemicals were purchased from commercial sources and used without further purification. Diisopropylamine (DIPA>99.5%) and tRNAs (specifically phenylalanine and total tRNA from brewer's yeast) was obtained from Sigma-Aldrich (St. Louis, Missouri). Formic acid (98-100%) was purchased from Merck (Darmstadt, Germany). 1,1,1,3,3,3-hexafluoro-2-propanol (HFIP, 99%) and N,N-diisopropylethylamine (DIPEA, 99%) were purchased from Thermo Fisher Scientific (Waltham, MA). All other chemicals were obtained from Sigma-Aldrich unless indicated otherwise.

Workflow of De Novo Sequencing of tRNA Mixtures

Each tRNA sample was divided into two portions. 10% was injected directly into the LC-MS instrument, while the remaining 90% was subjected to acid degradation before LC-MS injection. After obtaining LC-MS data sets of the control and acid-degraded portions, novel algorithms (available at https://github.com/rnamodifications/MLC-Seq) were used to identify each tRNA species or isoform and computationally separate its MS ladder fragments from the LC-MS data.

Controlled Acid Degradation of tRNA Sample

Formic acid was applied to degrade tRNA samples including yeast tRNA-Phe and mouse liver tRNA-Glu and tRNA-Gln (see “Mouse Liver tRNA Pulldown” section below) to produce MS ladders according to reported experimental protocols.^{20,21,23,26} Given that a single degradation time point may not yield a complete set of ladder fragments for tRNA sequencing, (1) a combination of different time points on split samples followed by repooling, and (2) mass ladder complementation (MLC) to account for any missing ladder fragments (See “Ladder Complementation and Generation of RNA Sequences” section below) is described. Each RNA sample solution was divided into three or more equal aliquots (each in an RNase-free thin-walled 0.2 mL PCR tube) and mixed with 50% (v/v) formic acid at 40° C. in a PCR machine. Typically, acid hydrolysis reactions were run for 2, 5, and 15 min, but additional time points (0.2, 0.5, 10, and 20 min) were included for sequencing yeast tRNA-Phe in order to obtain more complete ladders. Reaction mixtures were immediately frozen with dry ice at the specified times, followed by centrifugal vacuum concentration (Labconco Co., Kansas City, MO) to dryness. The dried samples from all time points were then usually combined and resuspended in 20 μL of nuclease-free water for the LC-MS measurement.

LC-MS Measurement and Processing of Intact and Acid-Degraded tRNA Samples

Each intact or combined acid-hydrolyzed tRNA sample was individually analyzed on an Orbitrap Exploris 240 MS (Thermo Fisher Scientific, Bremen, Germany) coupled to a Vanquish Horizon UHPLC using a DNAPac reversed-phase (RP) column (2.1 mm×50 mm, Thermo Fisher Scientific, Sunnyvale, California). Different solvent systems were employed in the separation of the samples. One solvent system used 2% HFIP and 0.1% DIPEA as eluent A pH 9 and methanol, 0.075% HFIP, and 0.0375% DIPEA as eluent B pH 9. The other solvent system used 1.7% HPIF and 0.15% diisopropylamine (DIPA) as eluent A, and 50% methanol with 1.7% HFIP and 0.15% DIPA as eluent B. Several different gradients were also used to separate both the intact and acid hydrolyzed samples. Sorter gradients were used for intact samples, whereas a longer gradient was used for formic acid hydrolyzed samples for better sample separation. A gradient of 20 to 80% B over 6.7 min or 30 to 80% B over 22 min was used for the analysis of intact RNA samples, while a gradient from 15 to 35% B over 20 min or 20 to 40% B in 19 min was used for acid-degraded samples. The flow rate was 0.2, 0.3, or 0.4 mL/min, and the separations were performed with the column temperature maintained at 60 or 70° C. Injection volumes were 3-25 μL, and sample amounts were 2-200 pmol of tRNA-Phe and 1 pmol (˜20 ng) of tRNA-Glu. tRNA samples were analyzed in negative-ion full MS mode from m/z 410 to 3200, m/z 550 to 2000, or m/z 600 to 2000, to obtain deconvoluted mass in the low or high region, with a scan rate of 2 spectra/s at 120 k resolution and m/z=200.

At least three samples, either intact or acid hydrolyzed, were repeated for LC-MS runs and analysis. The resulting LC-MS.raw files were deconvoluted and converted to Excel (.xlsx) files using Thermo BioPharma Finder 4.0-5.2 (ThermoFisher Scientific). The process underwent Intact Mass Analysis mode using the Xtract (isotopically resolved) deconvolution algorithm as previously described.^{20,21,23,26}

### Homology Search

Once LC-MS data are displayed as a two-dimensional (2D) t_{R}-mass plot, a homology search of intact tRNAs can be conducted in the monoisotopic mass range of >˜24 k Da using an in-house algorithm in Python (see GitHub). This algorithm identifies related tRNA isoforms that may share the same ancestral precursor tRNA but are different in absolute sequence, e.g., in posttranscriptional profiles of nucleotide modifications, editing, and truncations. Mass differences between two intact tRNA isoforms are calculated and matched to the known mass of nucleotides or nucleotide modifications in the database.²⁶ For example, a difference of 14.0157 Da (±10 ppm)⁴ can be assigned to a methylation (Me/-CH₂—) event, while a difference of 329.0525 Da corresponds to an additional A nucleotide. Therefore, these intact tRNAs are assigned to the same tRNA group and considered homologous isoforms of a specific tRNA for sequencing together.

The homology search is a nontarget preselection step to group possible related tRNA isoforms together for sequencing. However, only one monoisotopic mass difference is used to distinguish between two intact tRNA isoforms, which could lead to errors by including a tRNA isoform that does not belong to the specified tRNA group or omitting an isoform that does belong. These errors can be identified and corrected later when sequencing each group of tRNA isoforms, and sequencing results can further verify the interconnection between isoforms.

### Detection of Acid-Labile Nucleotide Modifications

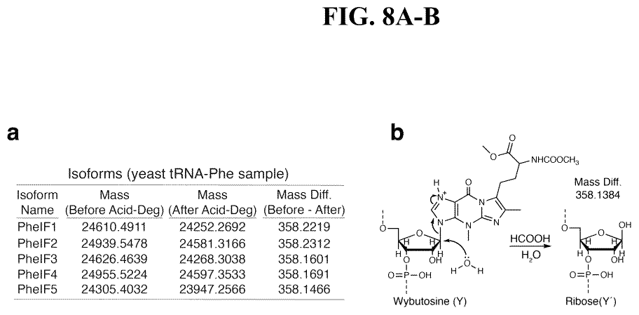

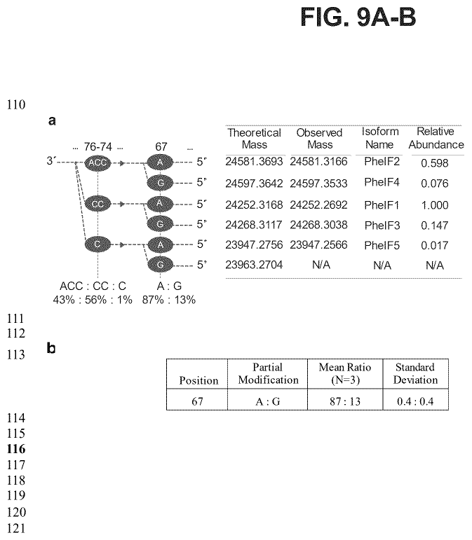

Acid-labile nucleotides are identified using another algorithm in Python (see GitHub) that analyzes the connections between the compounds (with a monoisotopic mass >24 kDa for RNAs) measured by LC-MS before and after acid degradation. For each such compound pair, if the monoisotopic mass difference can be matched to a known mass difference corresponding to a possible structural change to a nucleotide modification during acid hydrolysis (or the sum of several such changes), the compound pair will be selected and further considered to potentially contain acid-labile nucleotide modifications. In general, if the intact mass of the RNA species does not change after acid degradation, this intact mass will be used for MassSum data separation (see below). Otherwise, the presence of acid-labile nucleotides may be indicated by matching the observed mass difference with the theoretical mass difference caused by an acid-mediated structural change in a nucleotide or a combination of several such changes (see FIG. 8).

### 5′ and 3′ Ladder Separation

LC-MS analysis results in two different ladders, a 5′ ladder and a 3′ ladder, that can be computationally distinguished by the differences in their t_{R} values. This separates the data into two isolated but adjacent sigmoidal curves, one for each ladder. Due to the large number of fragment compounds, the dividing line between the 5′ and 3′ ladders is not obvious in the t_{R}-mass plot. Thus, a computational tool (see Github) was developed to separate the 5′ and 3′ fragments. It divides all compounds in each LC-MS data pool into two subgroup areas; compounds in the top collective curve of the t_{R}-mass plot are marked as 5′ fragments, while those in the bottom curve are marked as 3′ fragments. This should ideally identify as many fragments as possible while minimizing the number of fragments assigned to the incorrect ladder, although overlap between the two ladders may still occur if the t_{R} difference between fragments is very small. Some manual selection is also used, albeit to generate additional input fragments for the MassSum algorithm (see below) rather than as a primary means of separating 5′ and 3′ fragments.

### MassSum Data Separation

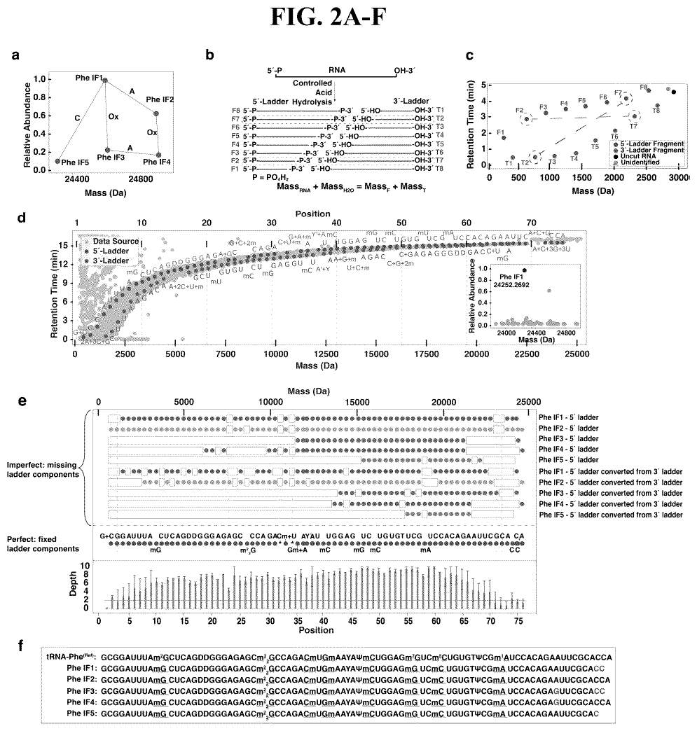

MassSum is an algorithm in Python (see Github) developed based upon the controlled acid hydrolysis of RNA presented in FIG. 2. MassSum takes advantage of the fact that the sum of the masses of each pair of fragments (5′ and 3′) produced from a single cut of an intact RNA is a constant value unique for each RNA isoform/species

$$\text{?} + \text{?} = \text{?} + \text{?} \qquad ( 1 )$$

$$\text{?} \text{indicates text missing or illegible when filed}$$

where mass_{intact} is the intact RNA, massy and mass' are the two fragment masses, and mass_{H2}O is the mass of one water molecule. This equation can be used to isolate ladder compounds corresponding to a specific isoform, which simplifies the data set by grouping MS ladder components into subsets, one for each tRNA isoform/species. MassSum operates by choosing two random compounds from the acid-degraded LC-MS data set and adding their masses; if the sum is equal to the mass of a known isoform/species, the fragments are selected into a subset corresponding to that isoform/species containing all its 3′ and 5′ ladders.

### GapFill

GapFill is another Python-based algorithm (see GitHub) developed to complement MassSum, which identifies pairs of corresponding 5′/3′ fragments and therefore cannot separate data if, e.g., there is no 5′ fragment found in the data that pair with a given 3′ fragment. GapFill was designed to “rescue” any ladder fragments missed by MassSum separation by first identifying gaps where fragments are missing from a ladder after the MassSum algorithm was applied and the corresponding values of mass_{low} and mass_{high}, the masses of, respectively, the heaviest known fragment below the gap range and the lightest known fragment above the gap range. The data set typically contains several fragments whose mass falls between mass_{low} and mass_{high}, but presumably none were selected by the MassSum algorithm during data separation. GapFill iterates over each fragment LC-MS data set whose mass falls within this range and examines the mass differences between this compound and mass_{low} and mass_{high}. If the mass difference is equal to the sum of one or more nucleotides or modifications in the RNA modification database,²⁶ it is noted as a connection. If the fragment in the gap has connections with both ending fragments, it is selected into a candidate pool for the subsequent sequencing process. After iteration, GapFill calculates connections of the fragments in the candidate pool and the frequency of each connection, and the fragments with the highest frequency are chosen to fill in the gap.

### Ladder Complementation and Generation of RNA Sequences

After MassSum and GapFill, each (RNA isoform has its own set of separate 5′ and 3′ ladders. If any ladder is perfect (i.e., without any missing fragments), the full RNA sequence can be read, from the first to the last nucleotide in the sequence. Incomplete ladders can be completed using other related isoforms to obtain a more complete ladder for sequencing. A Python-based computational algorithm (see Github) was designed to align ladders from related isoforms based on the position of the ladder fragment in the 5′/3′ direction. For example, FIG. 2E lays out the 5′ ladders for tRNA-Phe, positioned horizontally so that the nucleotide positions are aligned. Ladder complementation can be performed separately on 5′ or 3′ ladders (but not mixed ladders), resulting in one final 5′ ladder or one final 3′ ladder. Additionally, the 3′ fragments can be converted to their corresponding 5′ fragments for each tRNA isoform based on the MassSum principle. As such, each position in an tRNA isoform could have its original 5′ ladder fragment as well as a second fragment converted from the corresponding 3′ fragment, which can be used for confirmation and/or complementation.

### Stoichiometric Quantification of Partial Nucleotide Modifications/Editing

Stoichiometries of partial nucleotide modifications/alterations were quantified based on integrating EIC peaks corresponding to two or more fragments present at a single position in a sequence. EIC chromatograms were generated via BioPharma Finder 5.0-5.2 software (Thermo Scientific) using the Xtract (isotopically resolved) algorithm. In general, each EIC trace used a single m/z value corresponding to a fragment's most abundant isotope and the charge state z with the strongest MS signal; in cases where fragments at a single position had different preferred charge states, the preferred charge state for the more abundant fragment (i.e., with the greatest EIC area among all fragments of interest) was used. The ratio of EIC areas was taken as the relative abundance of their respective fragments. Each modification creates a branch in the MS ladder that is evident in all subsequent positions in the sequence, so this calculation was repeated in multiple positions for each partial modification to obtain six values that were used to calculate averages and standard deviations unless indicated otherwise.

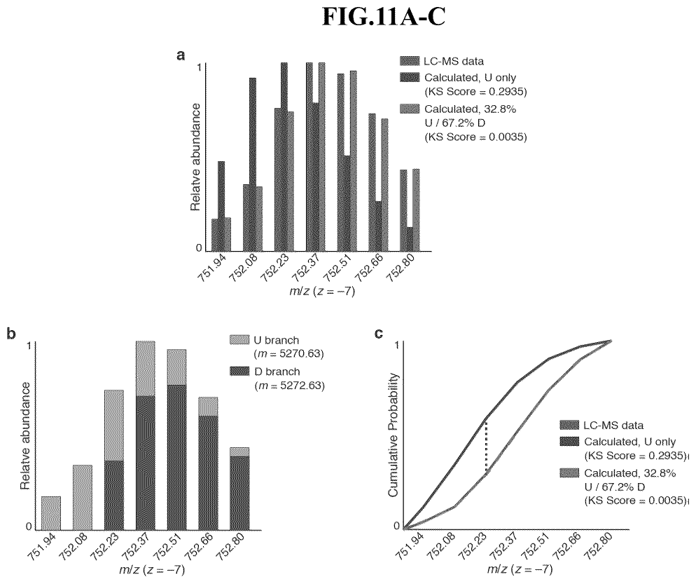

This process needed to be modified slightly in cases where partial components at a position were close in mass to each other (mass difference of 2 Da or less). In these cases, the isotopic patterns of the fragments overlapped significantly, such that the most abundant m/z values would feature contributions from both fragments. To address this, a composite isotopic pattern was calculated and compared to the pattern obtained from MS data. This is illustrated in FIG. 11 using results from position 16 of tRNA-Gln, which contains a partial U-D dehydrogenation. Theoretical single-component isotopic patterns were obtained using a Monte Carlo calculator taking 2×10⁶ samples and using the following isotope probabilities: P(¹³C)=0.0106; P(²H)=0.000145; P(¹⁵N)=0.03795; P(¹⁷O)=0.000385; P(¹⁸O)=0.002045. In most cases, results from the first seven m/z values were sufficient to represent the entire distribution. FIG. 11A shows the isotopic distributions of the obtained LC-MS data (obtained from EIC traces of each individual m/z value), a calculated distribution for U only, and an optimized composite distribution of 32.8% U and 67.2% D. FIG. 11B shows the breakdown of the composite distribution and its contributions from U and D branches at each m/z value. Stoichiometries were solved using a brute-force search to determine, to the nearest tenth of a percent, the composition whose theoretical composite isotopic pattern best matched the data pattern based on minimizing the Kolmogorov-Smirnov (KS) statistic between the two isotopic distributions; this statistic was used because its value is not dependent on the test sample size, making it easier to apply to MS data where the number of “observations” is ambiguous. FIG. 11C shows the cumulative probability functions for the LC-MS, U, and composite isotopic distributions, as well as the KS statistic for U only, i.e., the maximum distance between the theoretical and data-derived cumulative probability functions.

Mouse Liver tRNA Pulldown

The protocols described here were for bulk stock solution. The tRNA was enriched by an affinity pulldown assay combined with gel recovery, with modified protocols from a previous report.⁴⁶ The total RNA of mouse liver was harvested by TRIzol reagent (Invitrogen 15596026) according to the manufacturer's instructions. The concentration of the total RNA solution was adjusted to 2 mg/mL with RNase-free water. The small-RNA fraction (<200 nt) was separated in buffer containing 50% (w/v) poly(ethylene glycol) 8000 and 0.5 M NaCl solution by centrifugation at 12,000 rpm and 4° C. for 20 min. The supernatant was collected, followed by adding 1/10 volume sodium acetate (NaAc) solution (Invitrogen). One milliliter of supernatant was added to 3 mL of ethanol, and 5 μL of linear acrylamide (Invitrogen) was added to precipitate small RNAs (<200 nt) at −20° C. overnight, followed by centrifugation at 12,000 rpm at 4° C. for 20 min. The concentration of the small-RNA (<200 nt) solution was adjusted to 1 mg/mL, and 1 mL of the small-RNA solution with 6 μL of biotinylated probe (100 μM), 26 μL of 20× saline-sodium citrate (SSC) solution (Invitrogen) and 15 μL of RNase inhibitor (NEB) was incubated at 50° C. overnight. Streptavidin sepharose (200 μL, Cytiva 17511301) was added to the hybridization solution to enrich the biotin-labeled probe captured with the targeted tRNA. After incubation at room temperature for 30 min, the streptavidin sepharose was transferred to a 1.5 mL Ultrafree-MC tube (Millipore) and washed with 0.5×SSC buffer. The washing step was repeated 5 times. Then, 500 μL of nuclease-free water was added to the MC tube and incubated at 70° C. for 15 min, followed by centrifugation at 2500 g at room temperature for 1 min to elute the RNAs that are complementary to the biotinylated probe. The eluent was collected, followed by adding 1/10 volume NaAc solution. Then, 1 mL of eluent was added to 3 mL of ethanol, and 5 μL of linear acrylamide was added to precipitate RNAs at −20° C. overnight, followed by centrifugation at 12,000 rpm at 4° C. for 20 min. Nuclease-free water was added to dissolve the RNA pellets. RNA was loaded into a 7 M urea-PAGE gel for electrophoresis, and the main tRNA band was recovered from the PAGE gel as previously described³⁹ to obtain enriched tRNAs for MLC-Seq The DNA probe for the pull-down experiment was synthesized by Integrated DNA Technologies (IDT), and the sequence was as follows:

tRNA-Glu pulldown probe:
(SEQ ID NO. 41)
5′-Biotin-CTAACCACTAGACCACCAGGGA
tRNA-Gln pulldown probe:
(SEQ ID NO. 42)
5′-Biotin-TGGAGGTTCCACCGAGATTTGA

### Northern Blot

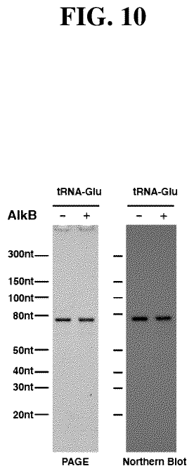

A Northern blot (FIG. 10) was performed as previously described⁸ to validate the captured tRNAs (as described above). RNA was separated on a 10% urea-PAGE gel stained with SYBR Gold, immediately imaged, transferred to a positively charged nylon membrane (Roche), and UV cross-linked with an energy of 0.12 J. Membranes were prehybridized with DIG Easy Hyb solution (Roche) for 1 h at 42° C. To detect tRNAs, membranes were incubated overnight (12-16 h) at 42° C. with DIG-labeled oligonucleotide probes synthesized by IDT. The membranes were washed twice with low-stringency buffer [2×SSC with 0.1% (wt/vol) SDS] at 42° C. for 15 min each, rinsed twice with high-stringency buffer [0.1×SSC with 0.1% (wt/vol) SDS] for 5 min each, and rinsed in washing buffer (1×SSC) for 10 min. Following the washes, the membranes were transferred into 1× blocking buffer (Roche) and incubated at room temperature for 3 h, after which antidigoxigenin-AP Fab fragments (Roche) were added into the blocking buffer at a ratio of 1:10,000 and incubated for an additional 30 min at room temperature. The membranes were washed 4 times with DIG washing buffer (1× maleic acid buffer, 0.3% Tween-20) for 15 min each, incubated in DIG detection buffer (0.1 M Tris-HCl, 0.1 M NaCl; pH 9.5) for 5 min, coated with CSPD ready-to-use reagent (Roche), and incubated in the dark for 30 min at 37° C. before imaging with a ChemiDoc MP Imaging System (Bio-Rad). Digoxigenin-labeled Northern blot probes for tRNA detection were synthesized by IDT, and the sequences were as follows:

tRNA-Glu Northern blot probe:
(SEQ ID NO. 43)
5′-DIG-CTAACCACTAGACCACCA
tRNA-Gln Northern blot probe:
(SEQ ID NO. 44)
5′-DIG-TGGAGGTTCCACCGAGATTT

Treatment of tRNA with AlkB

In total, 200 ng of tRNA were incubated in a 50-μL reaction mixture containing 50 mM Na-HEPES (pH 8.0; Alfa Aesar), 75 μM ferrous ammonium sulfate (pH 5.0), 1 mM a-ketoglutaric acid (Sigma-Aldrich), 2 mM sodium ascorbate, 50 μg/mL BSA (Sigma-Aldrich), 2.5 μL of RNase inhibitor (NEB), and 200 ng of AlkB enzyme at 37° C. for 30 min (the recommended mass ratio of AlkB enzyme to RNA is 1:1). The mixture was added to 500 μL of TRIzol reagent to perform the RNA isolation procedure according to the manufacturer's instructions.

Site-Specific Quantitative Analysis of Total tRNA Modifications

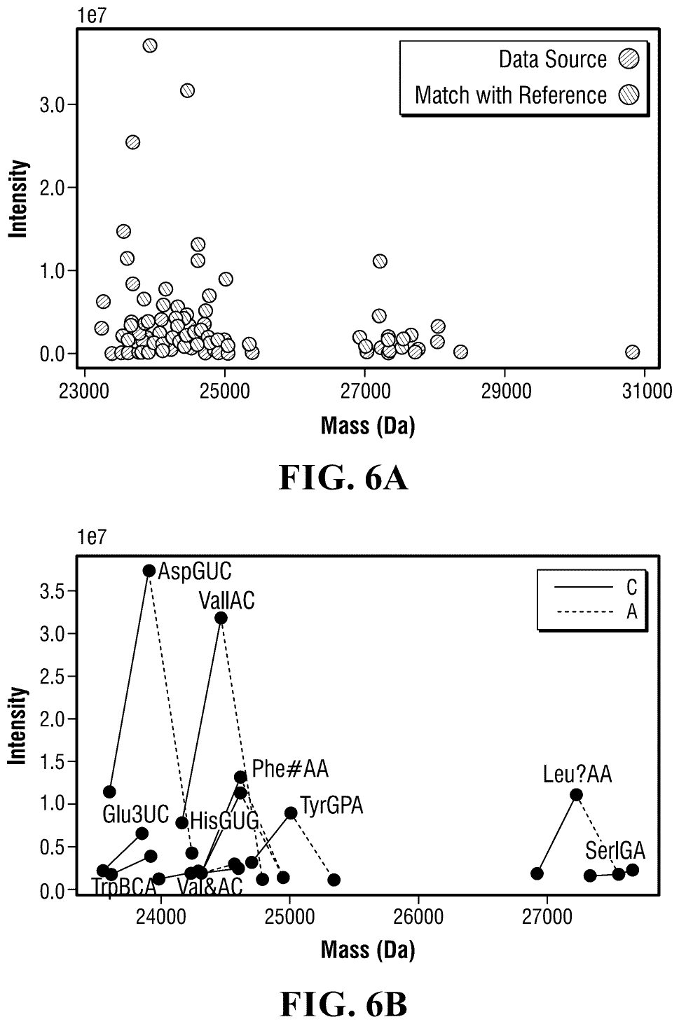

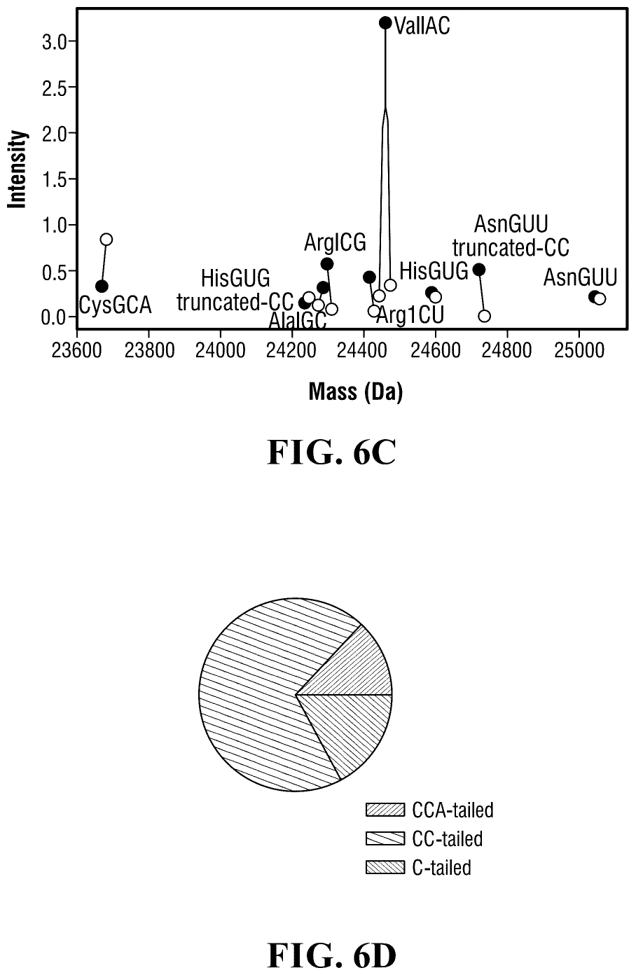

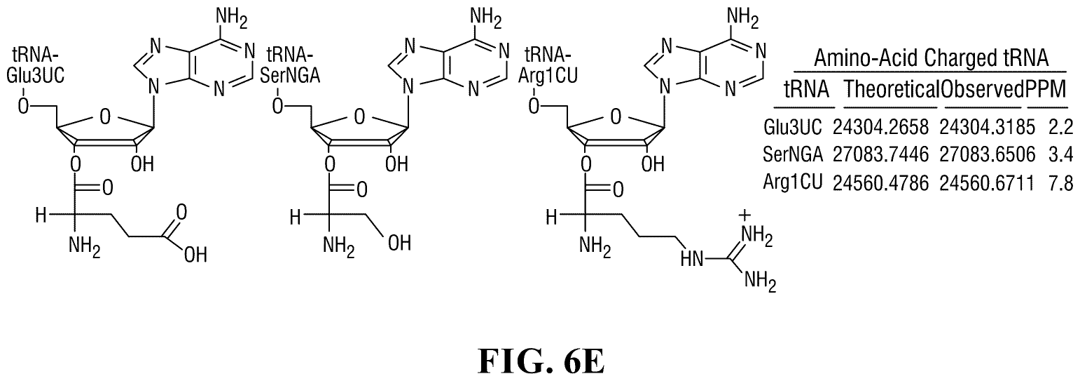

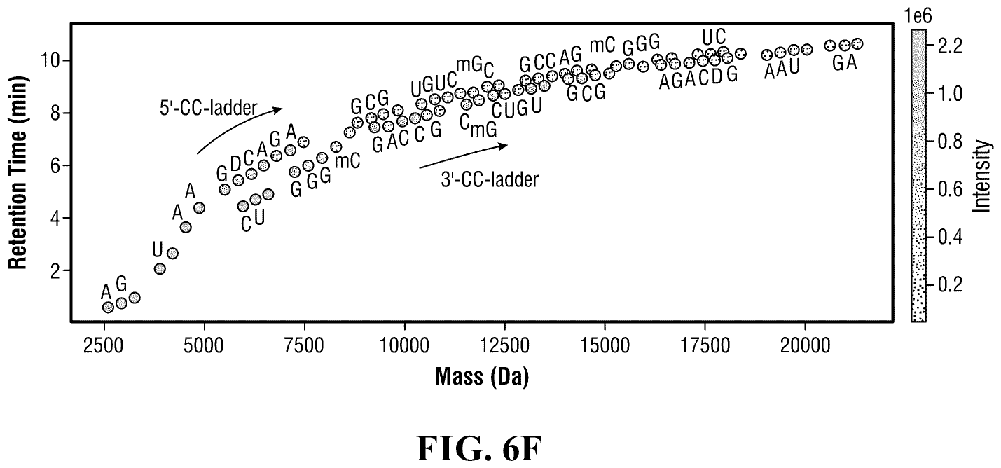

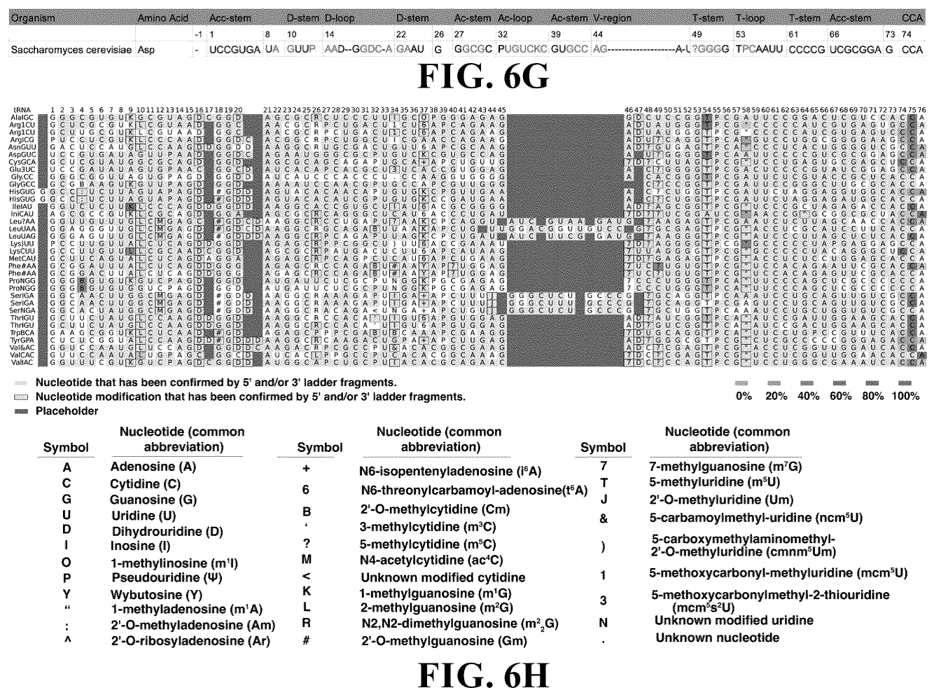

For each MLC-Seq experiment involving total yeast tRNA, approximately 10 pmol or 250 nanograms of total yeast tRNA was used. Of this amount, 10% was evaluated through LC-MS in its intact form without acid degradation, while the remaining 90% was degraded to produce ladder fragments for subsequent LC-MS measurement and sequencing analysis. To quantitatively map total RNA modifications in a site-specific manner, LC-MS data was used to create a 2D t_{R}-mass plot, similar to the homology section described earlier. An in-house Python algorithm (available on GitHub) conducted a homology search for monoisotopic masses above 24 kDa to identify intact tRNAs. Observed monoisotopic masses of intact tRNAs were compared to theoretical masses of tRNAs calculated using the tRNA sequence in the database. If there is a match between observed and theoretical masses, it indicates the identity of the specific tRNA type at the intact level. Additionally, the algorithm identifies differences in intact mass between observed tRNAs. A difference of 14.0157 Da (±10 ppm) indicates a possible partial methylation, while a difference of 329.0525 Da corresponds to an additional A nucleotide or a possible partial truncation event. For each observed monoisotopic mass, MassSum is performed to gather ladder fragments for the tRNA type, regardless of whether it matches the theoretical mass calculated from the tRNA database. Although some ladder fragments may be missing, the mass differences between two adjacent ladder fragments can be used for base calling and to read parts of the tRNA sequence de novo. The sequence segments, along with the location information on each nucleotide, are then used to search (via BLAST) the tRNA database and identify the specific RNA type at the ladder fragment level. Once the tRNA type is identified, its sequences and modifications are used to find all ladder fragments that may have been missed in the MassSum data separation step, and to construct the sequences and modifications shown in FIG. 6H. This also allows for verification of partial sequence modifications/editing at the fragment level in cases where this information cannot be extracted de novo from LC-MS data. While not all tRNA types can be detected at the intact level and be matched during the homology search step, it is still possible to identify the specific tRNA subtypes and their complete sequences using BLAST tRNA databases with parts of the de novo sequence results. These results are also listed in FIG. 6H. Nucleotides or modifications in each position of a tRNA type are confirmed either by its 5′ end or by one or more of its 3′ ladder fragments. However, depending on the abundance and detected ladder fragments for each tRNA, discrepancies can occur when confirming the first few nucleotides at the 5′ end of different tRNA types, even if they share identical nucleotides. For example, the C at the first position of tRNA-ProUGG was not confirmed, while the C at the first position of tRNA-TyrGPA was confirmed.

### Differentiating Pseudouridine and Uridine

While pseudouridine (Y) can be distinguished from U with N-cyclohexyl-N′-(2-morpholinoethyl)-carbodiimide metho-p-toluenesulfonate (CMC), as the CMC adducts of Ψ and U differ in mass.^{20,21} This study did not incorporate CMC treatment step, and therefore could not differentiate Ψ and U.

tRNA Position Numbering

To maintain consistency with published tRNA research, conventional numbering was adhered to as recommended by the cited reference.⁴⁸ For instance, the first nucleotide of the T-loop is consistently labeled as position 54, regardless of its actual sequence position in the tRNA, unless stated otherwise.

### Results

Although the examples presented below demonstrate the de novo sequencing of particular tRNA using refined tRNA samples, the MLC-Seq can also be used for quantitative analysis of total RNA samples containing all tRNA subtypes. Portions of each tRNA sequence can be read out de novo using the previously described methods, although the complexity of the LC-MS data for such a diverse sample currently prevents complete sequencing. Instead, the method is combined with known reference sequences to identify each tRNA ladder fragment from LC-MS data and construct ladders for tRNA sequence and modification analysis.

### Overview of MLC-Seq

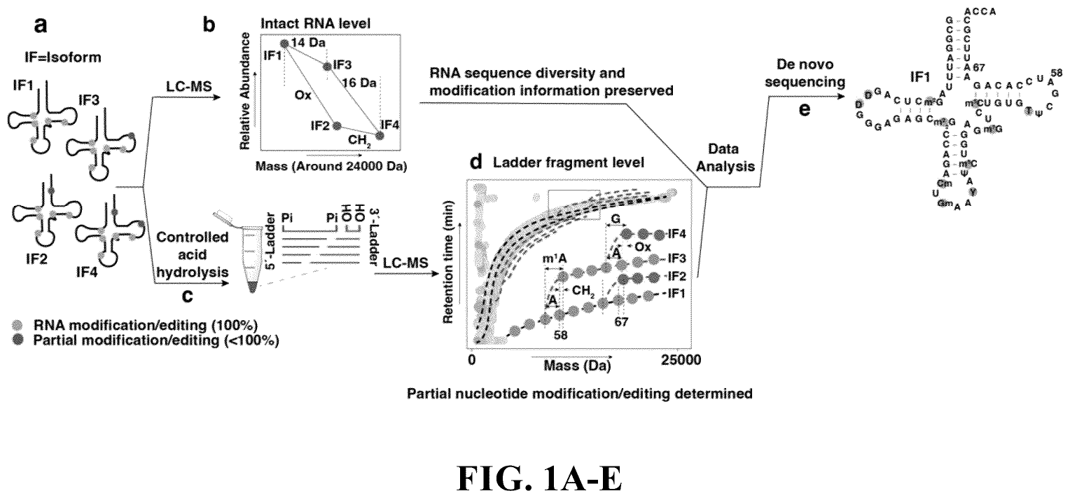

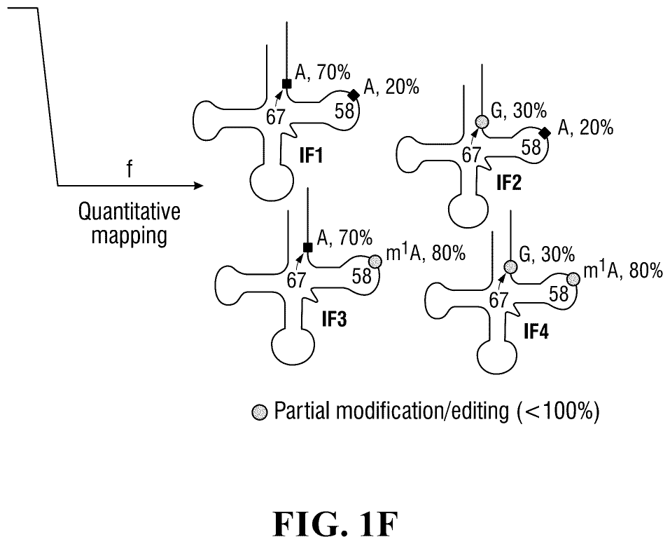

FIG. 1 outlines the MLC-Seq process, where ˜10% of a sample is analyzed intact without any treatment to preserve RNA sequence diversity and modification information (FIG. 1A-B), while the remaining 90% is subjected to controlled acid hydrolysis, which converts intact RNA into ladder fragments (FIG. 1B). Like previous MS-based sequencing work,²⁹-23 MLC-Seq relies on a set of ladders (one each in the 5′ and 3′ directions) to determine the identity, location, and any modifications for each nucleotide in RNA. Each ladder contains a series of fragments, each containing one more nucleotide than the previous fragment in the ladder (FIG. 1C). The mass differences between successive fragments functions as a general identifier for base calling nucleotides, including modified ones, while the ladder itself gives the order of nucleotides based on their increasing masses and t_{R} values.

The previous MS-based method, however, could not directly sequence full-length tRNA-Phe. The tRNA-Phe sample contained five isoforms, labeled Phe IF1-IF5 (FIG. 2), that cannot be physically separated and differ from each other by as little as a single nucleotide or modification. Furthermore, none of these isoforms yielded a perfect MS ladder (FIG. 2D), which was required by previous MS sequencing. MLC-Seq addresses the “imperfect ladder” problem by combining ladder fragments from coexisting isoforms of the same specific tRNA species. Missing fragments from one isoform ladder can be filled based on other isoform data to obtain complete ladders, allowing for direct sequencing of the full length tRNA-Phe.

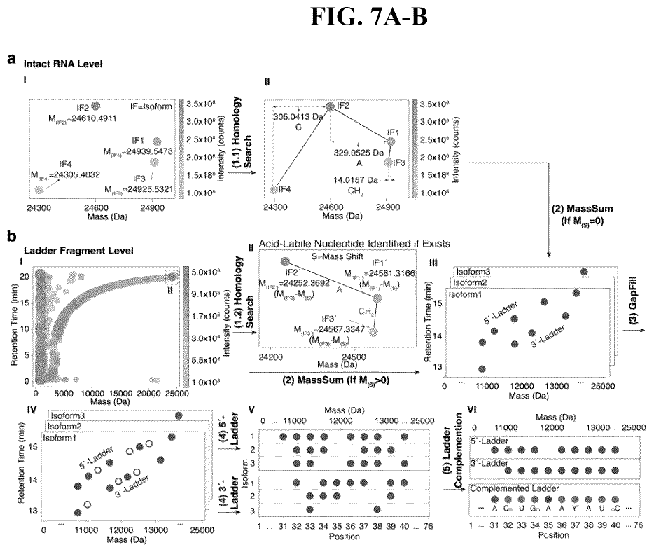

To obtain complete sequences, MLC-Seq incorporates a series of innovative bioinformatic tools to systematically process the MS data. First, a homology search algorithm developed in house measures the masses of all intact RNAs before and after acid degradation and groups related species together for sequencing based on known mass differences matching a nucleotide or modification. Thus, the homology search reveals not only the number of isoforms but how they differ from each other. The tRNA-Phe data in FIG. 2A indicates a 3′-terminal truncation, with Phe IF2 and Phe IF4 having the longest sequences and the others losing one (Phe IF1 and Phe IF3) or two (Phe IF5) nucleotides at the 3′end. Isoform pairs with a mass difference of ˜16 Da indicate a possible partial A-G transition, as their compositions differ by one oxygen atom. These masses are also compared to the masses of intact tRNA molecules found in acid-degraded samples (see FIGS. 7-8 and Table 1). The values will often be identical; any change in mass indicates the presence of acid-labile nucleotide modifications. This particular sample shows a mass decrease of ˜358.14 Da for all isoforms after acid hydrolysis. This is consistent with a single wybutosine (Y) nucleotide, which converts under acidic conditions to its depurinated ribose form (Y′) (FIG. 8B). This step does not reveal the location of any acid-labile nucleotide modifications, but confirming the presence (or absence) of such modifications is useful when subsequently processing MS data, identifying fragments, and constructing the ladder, which ultimately provides the location of all nucleotides, acid-labile or not.

While tRNA isoforms cannot be physically separated, the complex LC-MS data can be computationally separated into different isoforms and ladders. A novel MassSum algorithm was developed that utilizes the fact that the combined mass of any set of paired fragments generated by each single cleavage of a phosphodiester bond in the acid-mediated hydrolysis step is constant and equal to the sum of the mass of the intact RNA plus the mass of a water molecule²⁶ (FIG. 2B). Paired 5′ and 3′ fragments originated from the same RNA sequence can therefore be isolated from the MS data because each distinct full-length RNA sequence has a unique and constant mass (FIG. 2C-D).

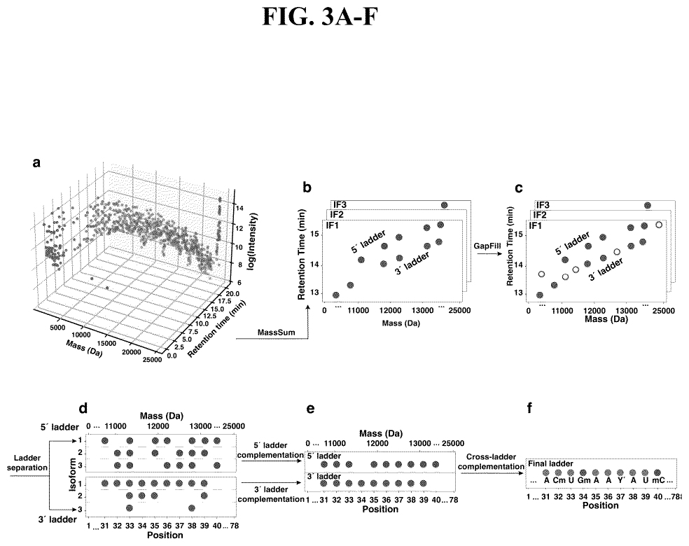

MassSum requires the presence of two paired 5′/3′ fragments generated by each single cleavage of a phosphodiester bond in the acid-mediated hydrolysis step to be identified. A GapFill algorithm was developed to subsequently fill in fragments in the 5′ or 3′ ladders missed by the MassSum data separation step when neither half of a 5′/3′ fragment pair was found (FIG. 3B). 5′ and 3′ ladders can be computationally separated from each other based on the sigmoidal curve that each 5′ and 3′ ladder displays in the t_{R}-mass plot (FIG. 3C). When a perfect 5′ or 3′ ladder exists for a sequence, it can be read out via base calling of each nucleotide or modification as described in previous literature.²⁰⁻²² If ladders are still missing fragments, they can be combined with ladders from coexisting related isoforms of the same tRNA group. Fragments missing from one tRNA isoform may be complemented by counterpart fragments from a related tRNA isoform (FIG. 3D-E). Gaps in a 5′ ladder can also be filled based on reversing the corresponding 3′ ladder, and vice versa (FIG. 3F).

MLC-Seq was first applied to sequence the full-length tRNA-Phe (FIG. 2), resolving the “imperfect ladder” problem and allowing for direct sequencing of all five tRNA isoform sequences, including three less abundant ones (Phe IF2,3,4 and 5; FIG. 2A). The results are consistent with reported tRNA-Phe sequence and simultaneously reveal various modifications.²¹ The reported RNA-Phe sequence with uridine (U) in the sixth position and adenosine (A) in the 67th position was identified. However, the tRNA-Phe sequence with cytidine (C) in the sixth position and guanosine (G) in the 67th position, predicted by yeast genome,⁴³ was not observed in the intact and acid hydrolyzed sample, likely due to its presence being below the limit of detection of the mass spectrometer (Table 1).

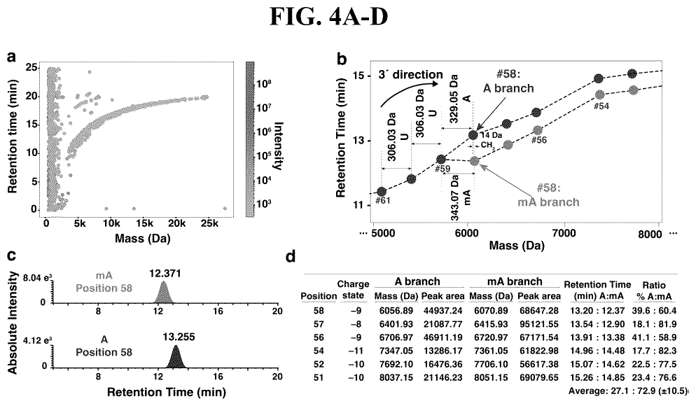

Isomeric nucleotides can be distinguished by applying an extra step prior to or following MS sequencing.²¹ Pseudouridine (Ψ) can be distinguished from uridine (U) with N-cyclohexyl-N′-(2-morpholinoethyl)-carbodiimide metho-p-toluenesulfonate (CMC), as the CMC adducts of Ψ and U differ in mass. Methylated positions can be identified using AlkB, a demethylating enzyme whose reactivity is selective based on the methylated nucleotides. Additionally, 2′-O-methylations can often be identified based on the fact that methyl groups in this position tend to block the formic acid hydrolysis of the phosphodiester bond. Thus, these fragments do not appear in the MS data, resulting in ladders with a missing fragment. This can be observed in FIG. 4B, where a missing fragment indicates the presence of a 2′-O-methylation.

### Preserving RNA Sequence Diversity and Modification Information

RNA samples often contain multiple types of RNA molecules corresponding to different RNA sequences or isoforms, and a key advantage of MLC-Seq is its ability to provide detailed results regarding sample diversity. It can distinguish between sequences, even those that differ by only a single nucleotide or modification, and quantify the relative ratio of different RNA sequences and further stoichiometry of partial modifications in the RNA sample. This is aided by advanced analytical instrumentation, including a Vanquish Horizon UHPLC coupled with an Orbitrap Exploris 240 MS (Thermo Fisher Scientific) that can measure the mass of intact tRNA up to ˜25 kDa without the need for a TI digestion step. Partial T1 digestion breaks full tRNA sequences into smaller fragments (˜35 nt), which complicates the data analysis but would be necessary for instruments with a lower mass resolution.²¹ MLC-Seq also reduces the necessary sample loading by 3 orders of magnitude, to roughly 20 ng (1 pmol of tRNA), which allows for sequencing of heterogeneous cellular tRNA.

The ability to measure the mass of intact full-length tRNAs makes it possible to observe differences in intact RNA molecules, indicate the existence of partial RNA modifications/editing, and further identify types, locate sites and quantify stoichiometries of these RNA modifications/editing. This can be observed in FIG. 4 using data from sequencing RNA-Glu (which is discussed in further depth in the following section). After acid degradation, the 5′ and 3′ ladders for tRNA-Glu form sigmoidal curves on a t_{R}-mass plot (FIG. 4A). A branch in this curve indicates a partial modification at this position, as illustrated in FIG. 4B 20,21,26 The mass difference between the branches indicates the nature of the partial modification; the 14 Da difference here indicates a partial methylation, where a canonical nucleotide in the lighter isoform is replaced in the heavier isoform by a methylated form of that nucleotide. Meanwhile, the position at which the branch begins can pinpoint the location of the partial modification in the sequence. Quantification of partial modifications can be calculated using the extracted ion chromatograms (EICs) from LC-MS raw data for each fragment²⁰ (FIG. 4C); the ratios of the EIC peak areas correspond to the stoichiometry of m¹A vs A at position 58 of tRNA-Glu (FIG. 4D). Previous results have shown that for sufficiently and equally long RNA (>7 nt), the addition of a single nucleotide modification does not significantly affect instrument response and that the ratio of EIC peak areas corresponds directly to the stoichiometry of partial modifications.

Direct Sequencing of Full-Length tRNA and Quantitative Mapping of Multiple Modifications

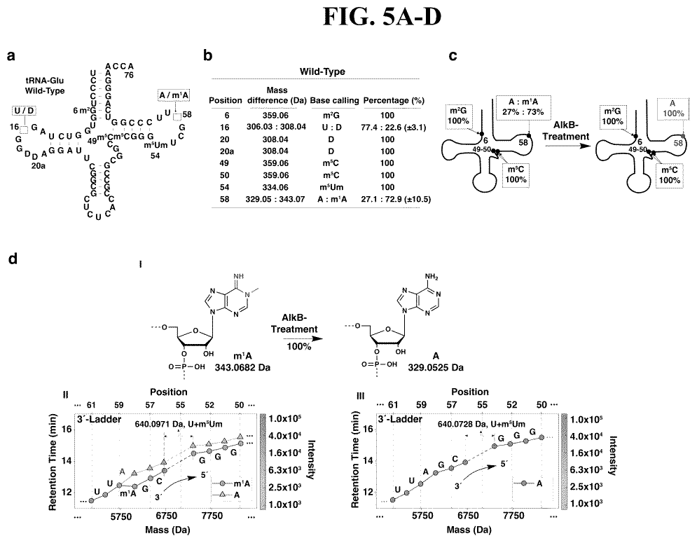

After verification on yeast tRNA-Phe, MLC-Seq was used to sequence a tRNA enriched from mouse liver, tRNA-Glu (FIG. 10). MLC-Seq was able to obtain complete sequence information for the tRNA, including the identity and position of all nucleotides (modified and canonical) and the site-specific stoichiometry of partial modifications and editing, marking the first instance of direct sequencing of full-length tRNAs with this level of detail. This is especially noteworthy as the modification-rich nature of tRNA has frustrated sequencing efforts in the past. The complete sequencing results of tRNA-Glu are shown in FIG. 5.

The MLC-Seq obtained sequence for tRNA-Glu is almost identical to that reported previously,^{37,38} although there are some significant differences in nucleotide modifications. It detected partial modifications at positions 16 and 58 of tRNA-Glu (FIG. 5A-B). Position 16 was partially hydrogenated, containing 77.4% U and dihydrouridine (D) in the remaining 22.6% (±3.1%). MLC-Seq found m¹A (1-methyladenosine) coexisting with A (adenosine) (72.9% m¹A and 27.1% A, ±10.5%) at position 58, which was previously thought to contain only A. Additionally, it determined that position 6 contained methylated G (guanosine) and position 10 contained canonical G (these were reversed in previous studies) and that the nucleotide modification at position 20a is D rather than the previously reported P′. Additional information on site-specific quantification of partial modifications in the tRNA can be found in Materials and Methods section and FIG. 11.

### Tracking Stoichiometric Changes in RNA Modifications

A powerful application of MLC-Seq is tracking changes in RNA modifications. These changes can be caused by diseases or cellular disturbances whose progression can be tracked through monitoring the identity and stoichiometry changes in RNA nucleotides (modified or canonical). To examine this, the mouse liver RNA sample was treated with AlkB to leverage its selectivity toward specific isomeric methylated nucleotides (FIG. 5C-D).^{39,40} AlkB reacts with m¹A, m¹G, and m³C (converting them to their respective canonical nucleotides) but is inert toward m⁶A, m²G, and m⁵C. Measuring site-specific changes in modification stoichiometry can verify AlkB's reactivity toward specific methylated nucleotides while demonstrating the capacity of MLC-Seq to quantitatively track changes in tRNA samples site-specifically at the single-nucleotide level.

Position 58 of wild-type (i.e., pre-AlkB treatment) tRNA-Glu had a 73:27 ratio of m¹A to A but comprised 100% A after AlkB treatment (FIG. 5B-D). Other types of methylated nucleotides in the sequence saw little to no change as a result of AlkB. This validates MLC-Seq as a way of precisely measuring site-specific RNA modification dynamics, a quality that is currently difficult to study quantitatively. To clarify, AlkB treatment itself does not quantify ratios but distinguishes m¹A from other modifications at position 58 of tRNA-Glu. The proportions of modified m¹A vs canonical A at this position was determined by integrating EIC peak areas of their corresponding ladder fragments at the identical positions (FIG. 4; see Materials and Methods section), a standard method in LC-MS for relative quantification.^{21,41,42}

Site-Specific Quantitative Mapping of Total tRNA Modifications

The de novo sequencing of tRNA-Glu was conducted using refined tRNA samples. However, MLC-Seq can also be used for quantitative analysis of total tRNA samples containing all tRNA subtypes. Portions of each tRNA sequence can be read out de novo using the previously described methods, although the complexity of the LC-MS data for such a diverse sample currently prevents complete sequencing. Instead, the method is combined with known reference sequences to identify each tRNA ladder fragment from LC-MS data and construct ladders for tRNA sequence and modification analysis.

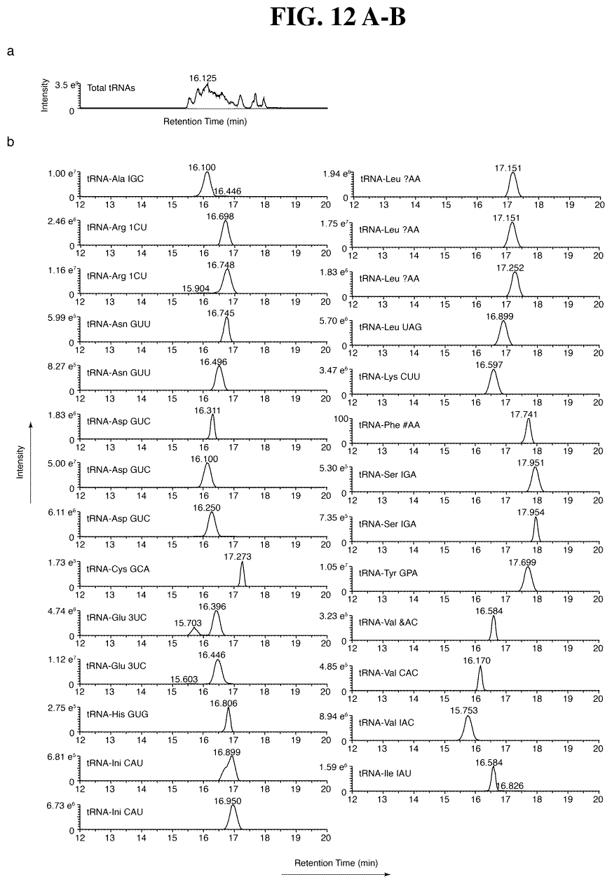

To manage the LC-MS data complexity of controlled acid-hydrolyzed yeast total tRNA samples (FIG. 12), the manuscript employs three functionalities of the MLC-Seq platform, including: (1) de novo sequencing (without sequence input) to identify tRNA types present in the samples, (2) site-specific mapping of tRNA modifications by cross-referencing between tRNA database sequences and the LC-MS data⁴³ and (3) site-specific quantification of partial tRNA modification stoichiometry. Different standards for ladder fragments are applied for each function. For de novo sequencing, a minimum of two successive ladder fragments is required for base calling any nucleotide or modifications. For mapping, ladder fragments are selected when their observed monoisotopic masses match those calculated from the corresponding database sequence. For stoichiometric quantification of partial tRNA modifications, at least two successive branch ladder fragments are required to both confirm the modification and determine the modification's stoichiometry. Additional details about total tRNA analysis are available in the Materials and Methods section.

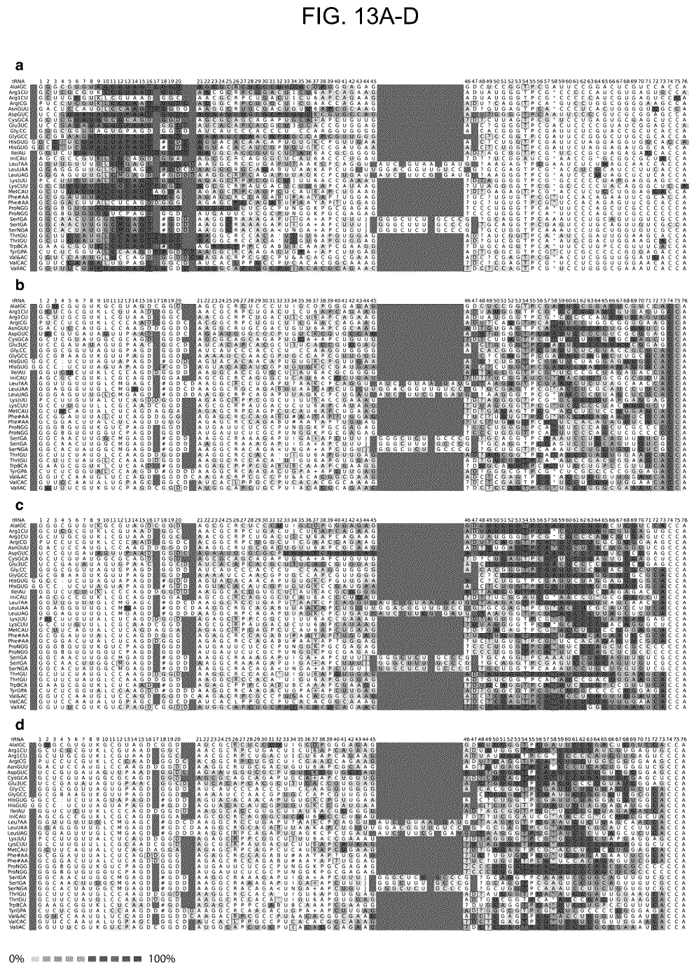

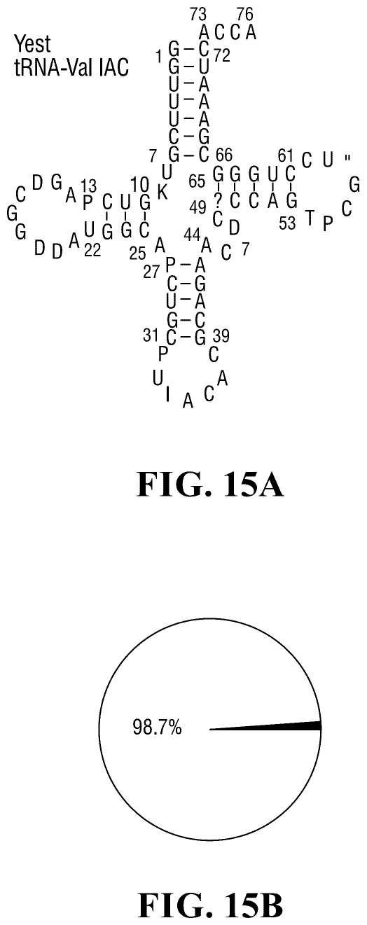

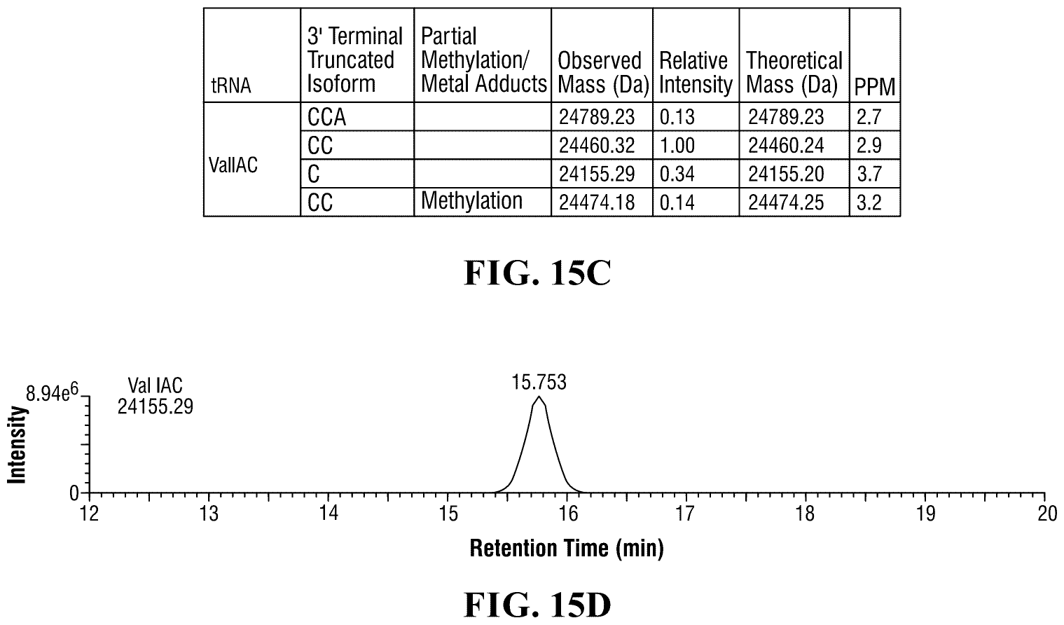

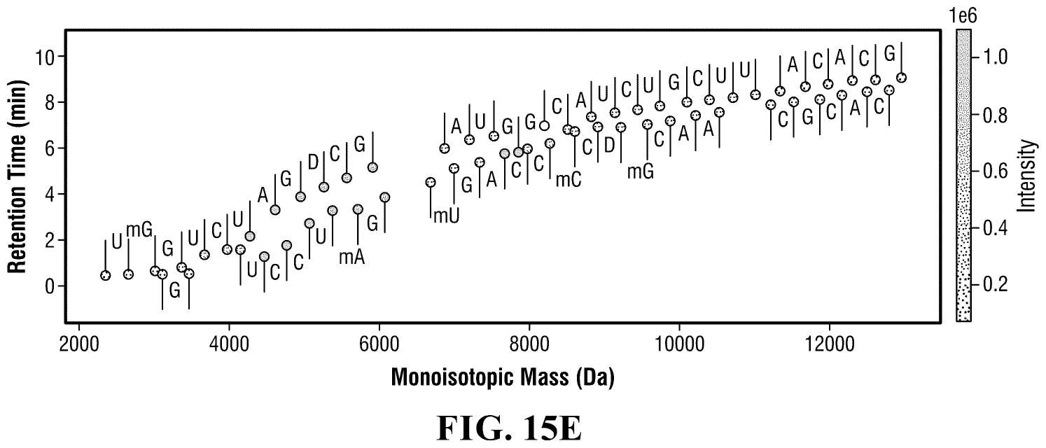

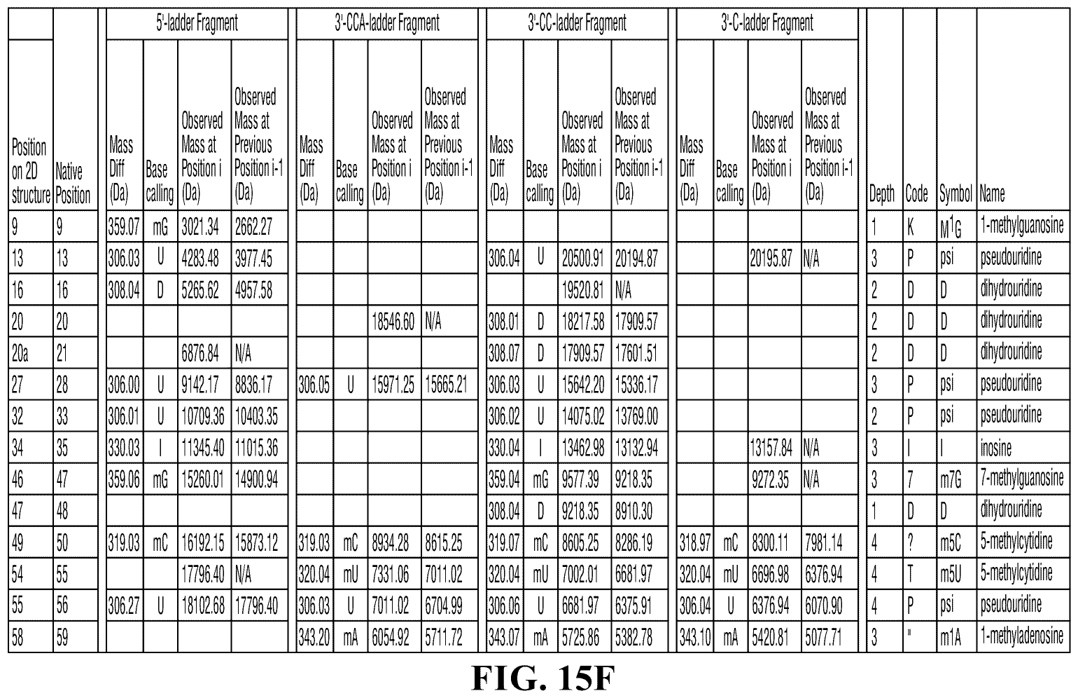

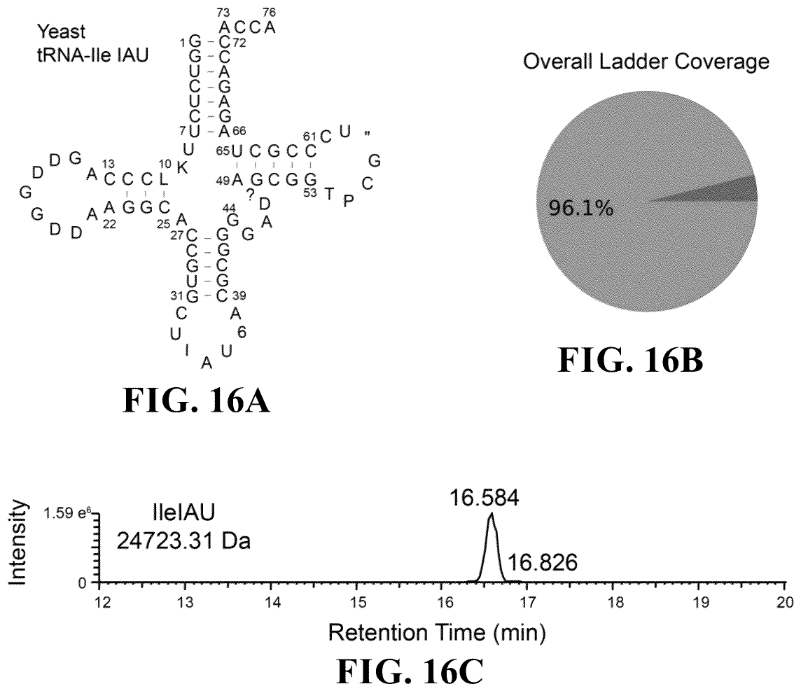

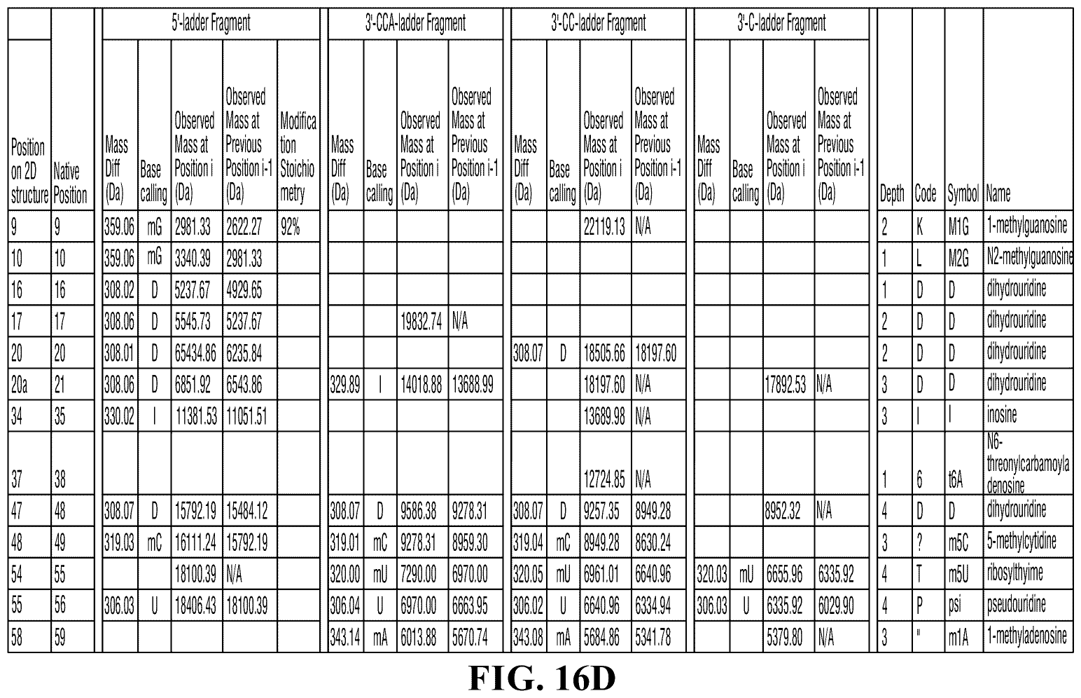

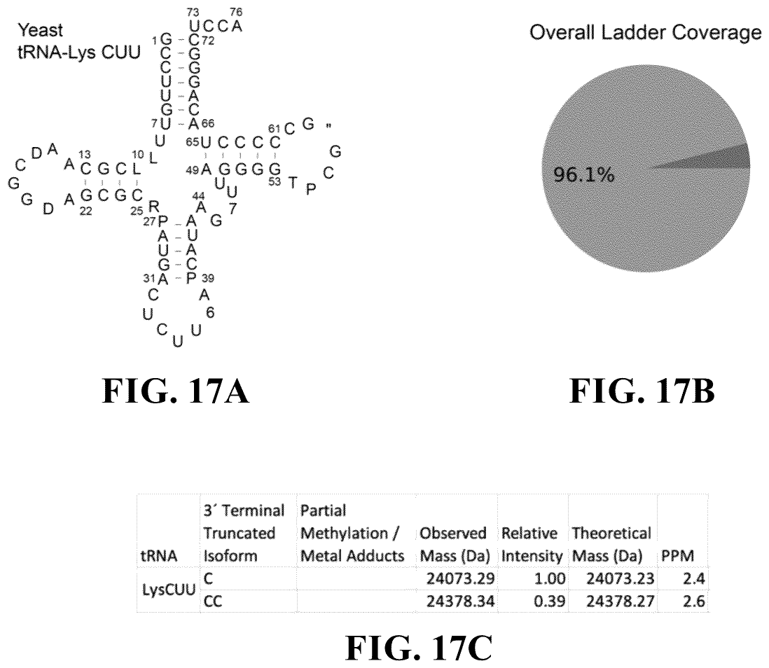

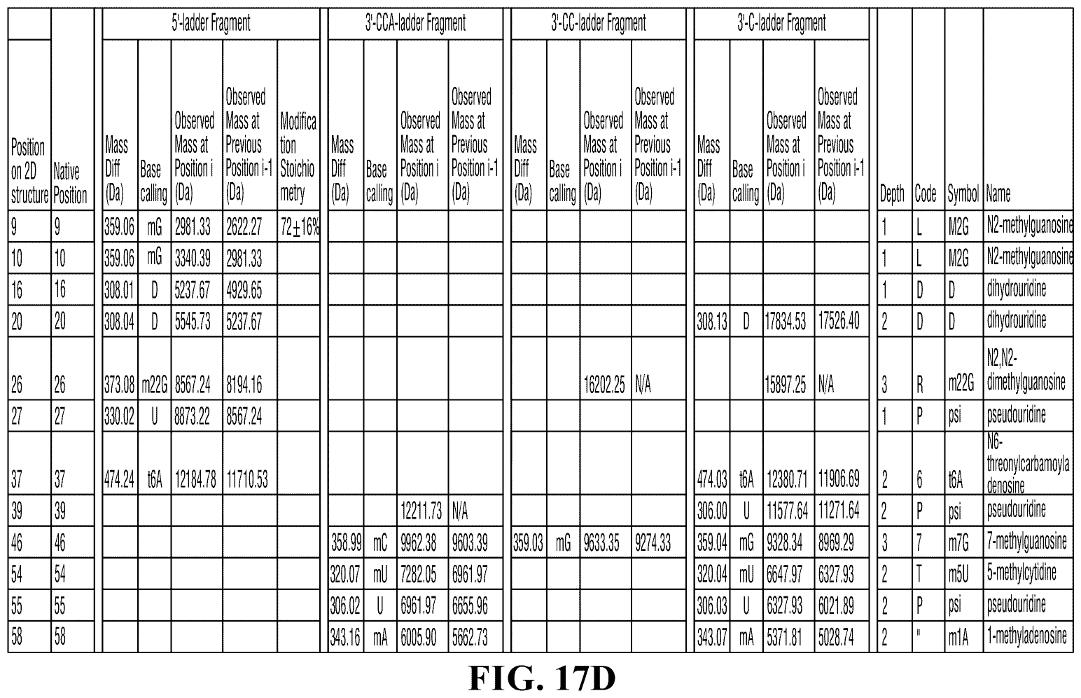

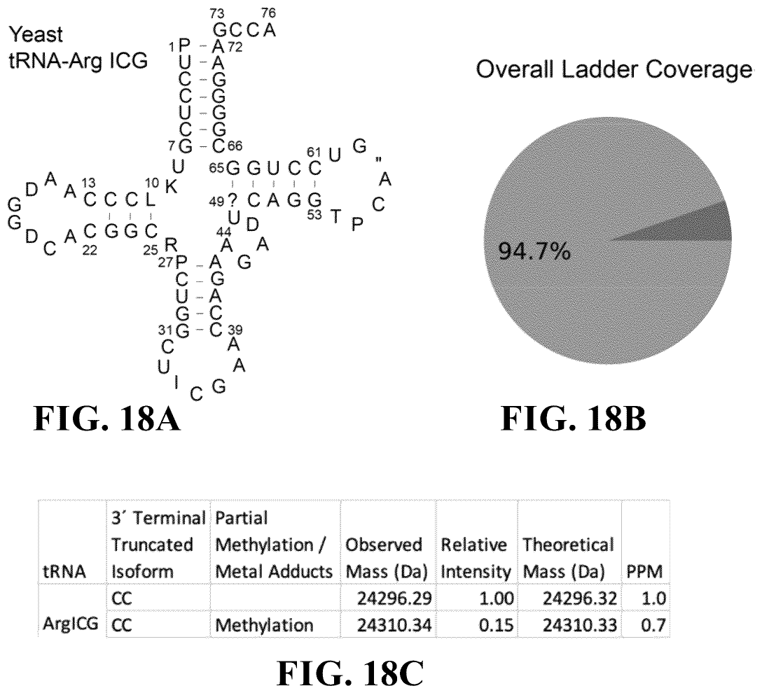

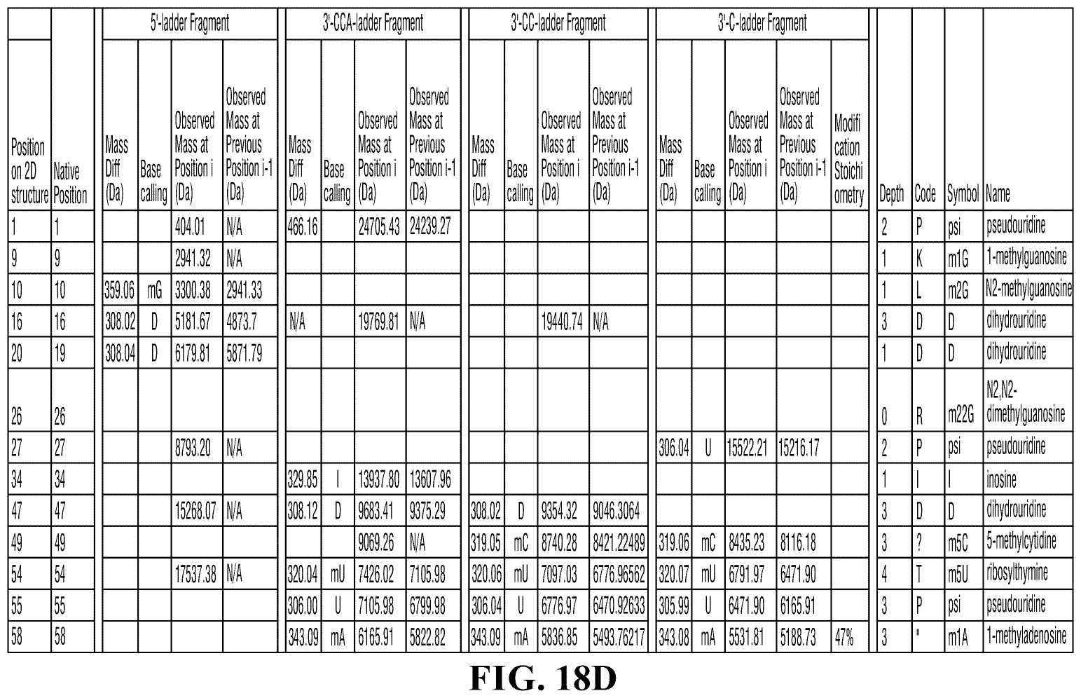

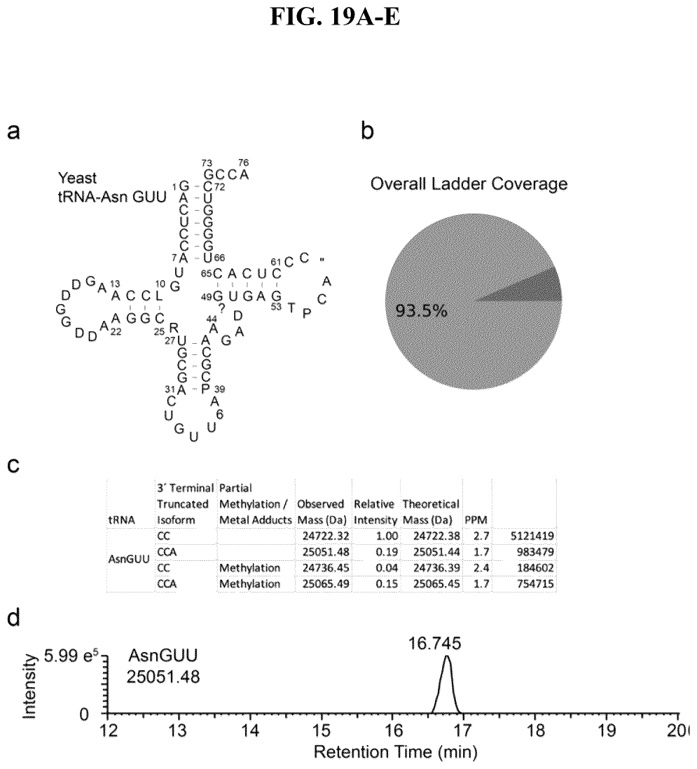

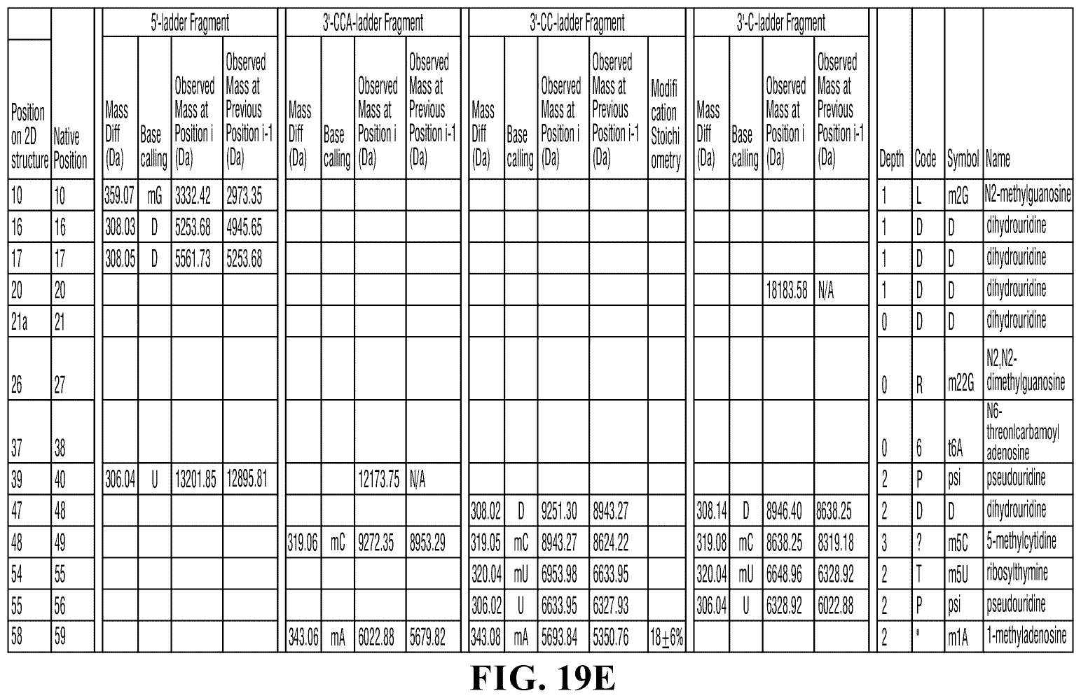

LC-MS analysis of intact molecules (FIG. 6A and Table 2) provides information on the number of different tRNA sequences in the sample; orange dots in this figure indicate that a match was found between the LC-MS data and the mass of a known tRNA sequence; these results can also be used to identify possible partial sequence modifications/editing such as 3′-end truncations (FIG. 6B) or methylations (FIG. 6C). A total of 64 matches were made to known tRNAs in a database³⁷ (FIG. 6A, FIG. 12-19, and Table 2), although it should be emphasized that this is a limitation of the reference data rather than the method itself.

FIG. 6D summarizes the portion of molecules showing-CCA, -CC, and -C 3′ ends. This further demonstrated the capacity of MLC-Seq to precisely quantify partial modifications and truncations, although it is possible that the 3′-end truncations observed are not inherent to the tRNAs but rather are the result of sample extraction and preparation.²¹ This is supported by the presence of the intact 3′ CCA in RNA-Glu extracted from mouse liver, shown in FIG. 5A. Intact molecule analysis can also indicate the presence of amino acid-charged tRNA molecules (FIG. 6E).

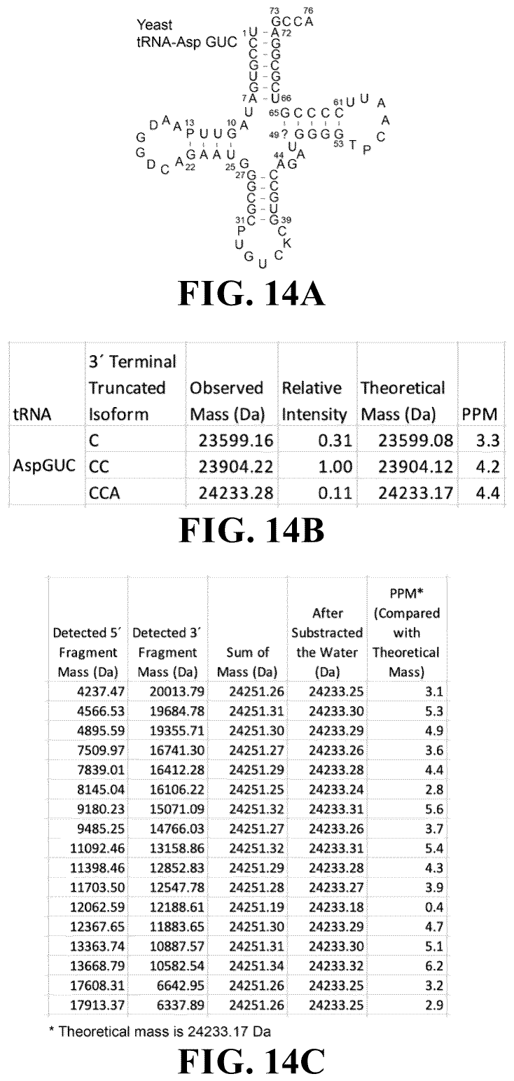

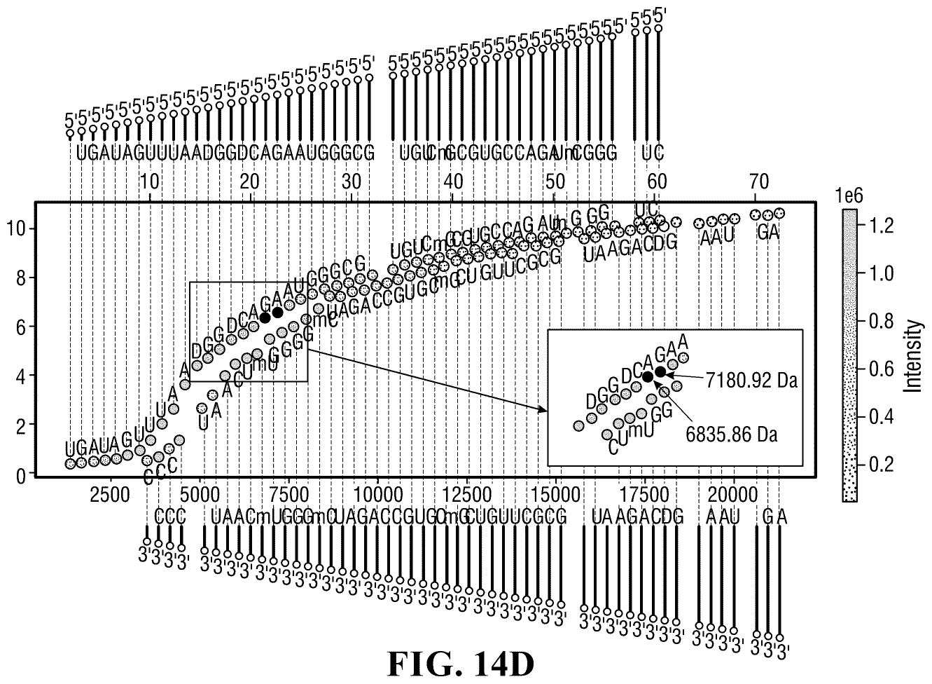

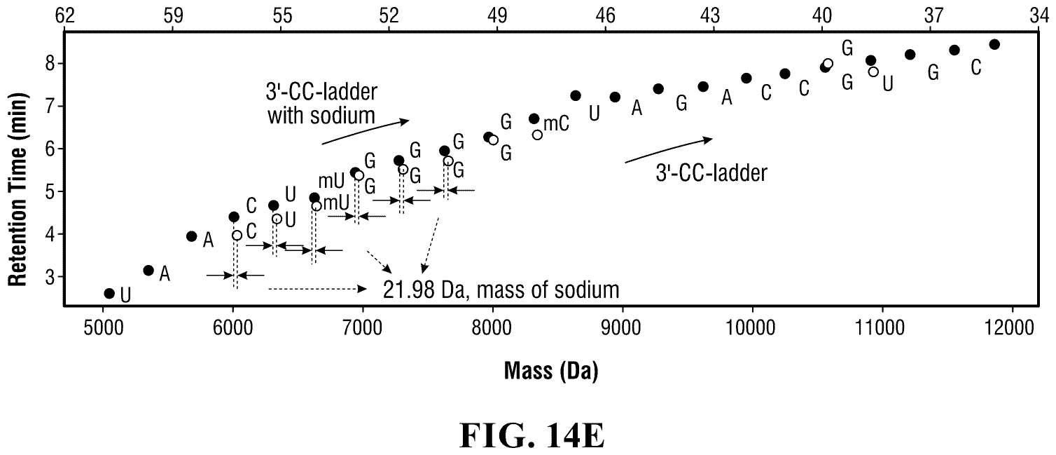

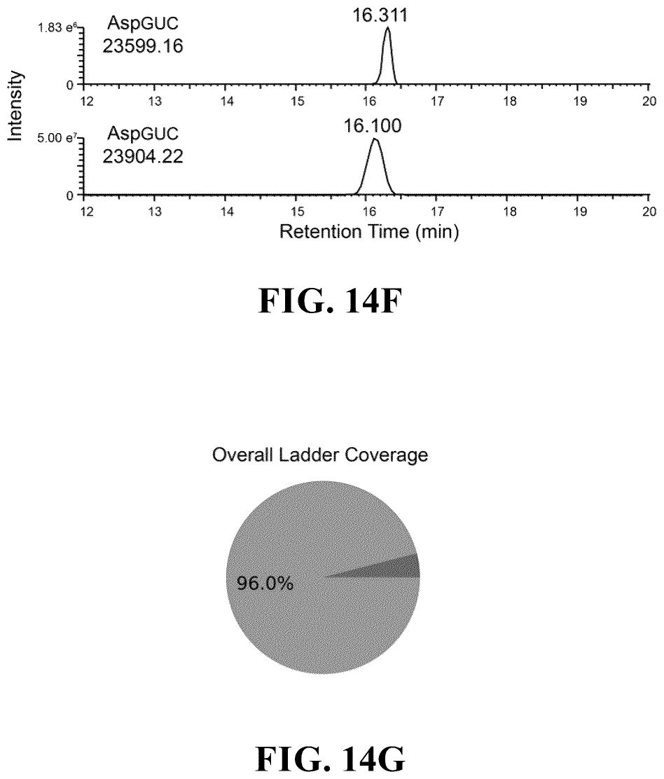

The sample is then subject to controlled acid degradation using the previously described methods. MassSum offers a relatively straightforward and largely automated process for finding matching 5′ and 3′ fragments and facilitating the identification of which tRNA subtype they correspond to. Partial sequences can be constructed de novo from this data (FIG. 6F) and used in the Basic Local Alignment Search Tool (BLAST)⁴⁴ to cross-reference between databases of known sequences and the LC-MS data (FIG. 6G). Matches between the two can confirm the sequences of all tRNA subtypes in the sample (FIG. 6H and Table 2), while data points not matching the known sequences may indicate partial modifications that can be further identified and pinpointed based on their intensity and mass difference relative to known sequence values. This method allows for thorough analysis and quantification of multiple RNA sequences in samples, including those of highly complex total RNA, while also offering site-specific identification and quantification of partially modified or edited nucleotides. It was able to reveal several new partial and complete modifications for multiple tRNA subtypes. For example, a partial m²G methylation in tRNA-LysCUU position 9 (72±16% coexisting with canonical G), a partial Cm modification at RNA-ProNGG position 4 (68±13%, coexisting with canonical C), and partial modified A at position 58 of both tRNA-ArgICU (20±6%) and tRNA-AsnGUU (18±6%) was identified. Further details on the partial nucleotide modifications in the total yeast tRNA sample—including identity, location, stoichiometry, as well as specific tRNAs and isoforms—are available in Tables 3-4 and FIG. 14-19.

MLC-Seq is a significant advance over previous de novo MS sequencing methods, which require a complete ladder to identify each nucleotide and its position in the sequence. This is particularly challenging when sequencing low abundance tRNA species or isoforms whose ladder fragments may only be present in quantities below the instrument detection limit. MLC-Seq circumvents the need for a perfect ladder, allowing MS sequencing to be performed for a broader range of RNA samples. This method surpasses conventional MS/MS-based analytical strategies, which often require prior sequence input, and provides an unbiased approach for comprehensive de novo direct sequencing of tRNA modifications, a challenge for other sequencing methods. Contrary to traditional MS sequencing which necessitates a highly purified homogeneous short RNA sequence (<˜30 nt long), MILC-Seq has the capacity to sequence each full-length tRNA type even within complex heterogeneous samples by segregating their MS ladder fragments. Algorithms in MLC-Seq can incorporate partial ladders with missing fragments, identify each RNA in a mixture, and computationally separate MS ladder fragments for sequencing of each (RNA, including those whose scarcity would prevent the acquisition of a perfect ladder.

However, for complex mixtures such as total RNA where incomplete ladders cannot be fully fixed, incorporating known tRNA reference sequences from databases during the sample analysis may be necessary.

Additionally, MLC-Seq allows simultaneous quantitative mapping of all nucleotide modifications with single-nucleotide stoichiometric precision, potentially enabling quantitative mapping of tRNA modifications in a tissue-specific manner. The presence of a branch point in a sigmoidal fragment ladder curve on a t_{R}—mass plot is indicative of a partial modification/editing. MLC-Seq can monitor changes in RNA modification dynamics and map modifications that have altered stoichiometry in different cellular and disease contexts. In contrast to cDNA-based RNA sequencing, which removes information on modifications, MLC-Seq preserves information regarding tRNA sample diversity (for visualizing each (RNA) and modification (for revealing modification type, location, stoichiometry, etc.).

It is notable that tRNA samples were typically mixtures of coexisting heterogeneous cellular tRNA sequences and isoforms that cannot be physically separated. It is believed that, contrary to common belief, it is not possible to obtain a single “pure” tRNA sequence, since there are always single-nucleotide sequence variations at different loci. This resonates with previously proposed ideas about the emerging complexity of the tRNA world.²⁸ This makes MLC-Seq convenient for tRNA samples containing multiple sequences or isoforms while remaining effective for single RNA species. It can also be used for quantitative analysis of unrefined total RNA when used in conjunction with a sequence database, providing site-specific information on the stoichiometry of partial tRNA modifications/editing.

The ability to computationally separate LC-MS data from different sequences or isoforms in a single sample increases the throughput of de novo MS-based RNA sequencing and facilitates large-scale MS sequencing of biological samples. By incorporating advanced instrumentation and greater automation, MLC-Seq could match the capacity of classic Sanger sequencing, making it possible to directly sequence RNAs and diverse modifications at a large scale. These methods could also be used to sequence longer RNAs (e.g., tRNA, snoRNA, snRNA, Y RNA, and vault RNA) and small noncoding RNAs (e.g., miRNA, piRNA, tsRNA, rsRNA, and ysRNAs) and as an orthogonal approach to verify results from high-throughput sequencing methods.

The documents listed below and referenced herein are incorporated herein by reference in their entireties, except for any statements contradictory to the express disclosure herein, subject matter disclaimers or disavowals, and except to the extent that the incorporated material is inconsistent with the express disclosure herein, in which case the language in this disclosure controls. Incorporation by reference of the following shall not be considered an admission by the applicant that the incorporated materials are prior art to the present disclosure, nor shall any document be considered material to patentability of the present disclosure.

TABLE 1
Five intact tRNA isoforms detected in a yeast tRNA-Phe sample
before acid 289 hydrolysis. This table presents five intact
tRNAs isoforms (~24 kDa average mass, ~80 nt 290 length)
detected from three LC-MS runs of the yeast tRNA-Phe sample.

| | Sample 1 | | Sample 2 | | Sample 3 | |
|---|---|---|---|---|---|---|
| Isoform | Mass | Intensity 1 | Mass | Intensity 2 | Mass | Intensity 3 |
| 1 | 24610.51 | 30078938 | 24610.49 | 16300000 | 0 | 0 |
| 2 | 24939.57 | 17267727 | 24939.55 | 96909195 | 24939.51 | 76926702 |
| 3 | 24626.48 | 4844897 | 24626.46 | 24644079 | 24626.43 | 9640439 |
| 4 | 24955.55 | 3119295 | 24955.52 | 14853915 | 24955.48 | 9010854 |
| 5 | 24305.42 | 885392.1 | 24305.41 | 2582277 | 0 | 0 |

TABLE 2
Intact tRNA monoisotopic masses detected in a yeast total tRNA sample before 297 acid
hydrolysis. This table presents monoisotopic masses of intact tRNAs (~24 kDa average 298 mass, ~80
nt length) detected by LC-MS in the yeast total tRNA samples. Although 129 distinct 299 intact
masses were detected, these do not correspond to 129 unique tRNA types. Various masses 300 are
attributable to isoforms arising from partial methylation (FIG. 6c) or 3′ end truncations (FIG. 301 6b
and 6d). Out of the 129 masses, only 64 match the calculated tRNA masses from the tRNAdb 302
2009 database and are highlighted in orange in FIG. 6a. For each matching tRNA, both observed 303
and theoretical masses are listed. Theoretical masses are calculated from the tRNAdb 2009 304
database. Each section focuses on a unique tRNA family and related isoforms, which may differ 305
in as little as a nucleotide or modification. Intensity normalization has been applied, setting the 306
highest tRNA intensity to a relative intensity value of 1. This standardizes the dataset's intensities 307
between 0 and 1. Notably, some tRNAs with significant intensities could not be sequenced due 308 to
the absence of matching entries in the current tRNAdb 2009 database. The data comprises all 309
monoisotopic masses detected across six independent LC-MS runs.

| | Monoisotopic Mass | Relative | | Isoform (3′ | Partial Modification | Theoretical Mass | |
|---|---|---|---|---|---|---|---|
| Index | (Da) | Intensity | tRNA | terminal) | or Adducts | (Da) | PPM |
| 1 | 24273.31 | 0.0331 | AlaIGC | CC | Demethylation | 24273.18 | 5.2 |
| 2 | 24616.41 | 0.0182 | AlaIGC | CCA | | 24616.25 | 6.4 |
| 3 | 24287.14 | 0.0865 | AlaIGC | CC | | 24287.19 | 2.2 |
| 4 | 24429.32 | 0.0164 | Arg1CU | CCA | Demethylation | 24429.30 | 0.7 |
| 5 | 24415.41 | 0.1143 | Arg1CU | CCA | 2 | 24415.29 | 4.9 |
| | | | | | Demethylation | | |
| 6 | 23809.16 | 0.0058 | Arg1CU | C | | 23809.22 | 2.2 |
| 7 | 24114.31 | 0.1587 | Arg1CU | CC | | 24114.26 | 2.3 |
| 8 | 23847.15 | 0.0295 | Arg1CU | C | K | 23847.17 | 1.2 |
| 9 | 24296.29 | 0.1519 | ArgICG | CC | | 24296.32 | 1.0 |
| 10 | 24310.34 | 0.0229 | ArgICG | CC | Methylation | 24310.33 | 0.7 |
| 11 | 24722.32 | 0.1372 | AsnGUU | CC | | 24722.38 | 2.7 |
| 12 | 24736.45 | 0.0049 | AsnGUU | CC | Methylation | 24736.39 | 2.4 |
| 13 | 25065.49 | 0.0202 | AsnGUU | CCA | Methylation | 25065.45 | 1.7 |
| 14 | 25051.48 | 0.0264 | AsnGUU | CCA | | 25051.44 | 1.7 |
| 15 | 23599.16 | 0.3058 | AspGUC | C | | 23599.08 | 3.3 |
| 16 | 24255.30 | 0.0569 | AspGUC | CCA | Na | 24255.16 | 5.8 |
| 17 | 23876.12 | 0.0520 | AspGUC | CC | 2 | 23876.10 | 0.8 |
| | | | | | Demethylation | | |
| 18 | 23904.22 | 1.0000 | AspGUC | CC | | 23904.12 | 4.2 |
| 19 | 24233.28 | 0.1119 | AspGUC | CCA | | 24233.17 | 4.4 |
| 20 | 23987.25 | 0.0325 | CysGCA | CC | Methylation | 23987.21 | 1.4 |
| 21 | 23682.22 | 0.2258 | CysGCA | C | Methylation | 23682.17 | 2.0 |
| 22 | 23668.20 | 0.0904 | CysGCA | C | | 23668.16 | 1.5 |
| 23 | 24288.16 | 0.0467 | CysGCA | CCA | Demethylation | 24288.25 | 3.6 |
| 24 | 23846.21 | 0.1740 | Glu3UC | CC | | 23846.12 | 3.5 |
| 25 | 23541.13 | 0.0572 | Glu3UC | C | | 23541.08 | 2.2 |
| 26 | 23241.13 | 0.1671 | GlyGCC | CC | Methylation | 23241.07 | 2.6 |
| 27 | 24241.18 | 0.0491 | HisGUG | CC | | 24241.22 | 1.5 |
| 28 | 24570.33 | 0.0771 | HisGUG | CCA | | 24570.27 | 2.7 |
| 29 | 24584.33 | 0.0594 | HisGUG | CCA | Methylation | 24584.28 | 2.3 |
| 30 | 23936.20 | 0.0051 | HisGUG | C | | 23936.17 | 1.2 |
| 31 | 24763.43 | 0.0526 | IniCAU | CCA | | 24763.48 | 1.9 |
| 32 | 24763.50 | 0.1842 | IniCAU | CCA | | 24763.48 | 0.8 |
| 33 | 24801.44 | 0.0057 | IniCAU | CCA | K | 24801.44 | 0.1 |
| 34 | 24472.24 | 0.0753 | IniCAU | CC | K | 24472.39 | 6.1 |
| 35 | 27220.71 | 0.2972 | Leu?AA | CC | | 27220.68 | 1.3 |
| 36 | 27549.76 | 0.0467 | Leu?AA | CCA | | 27549.73 | 1.1 |
| 37 | 26915.69 | 0.0484 | Leu?AA | C | | 26915.63 | 2.0 |
| 38 | 27541.71 | 0.0172 | LeuUAG | CCA | | 27541.66 | 1.8 |
| 39 | 27212.66 | 0.1188 | LeuUAG | CC | | 27212.61 | 1.9 |
| 40 | 24073.29 | 0.0507 | LysCUU | C | | 24073.23 | 2.4 |
| 41 | 24378.34 | 0.0197 | LysCUU | CC | | 24378.27 | 2.6 |
| 42 | 24939.54 | 0.0359 | Phe#AA | CCA | | 24939.51 | 1.4 |
| 43 | 24305.43 | 0.0508 | Phe#AA | C | | 24305.72 | 11.6 |
| 44 | 24610.51 | 0.3508 | Phe#AA | CC | | 24610.76 | 10.1 |
| 45 | 24610.39 | 0.3017 | Phe#AA | CC | | 24610.76 | 14.8 |
| 46 | 24321.33 | 0.0961 | Phe#AA | C | | 24320.73 | 24.6 |
| 47 | 27023.67 | 0.0183 | SerIGA | C | | 27023.63 | 1.4 |
| 48 | 27328.62 | 0.0406 | SerIGA | CC | | 27328.67 | 1.8 |
| 49 | 27657.75 | 0.0602 | SerIGA | CCA | | 27657.72 | 0.9 |
| 50 | 24170.24 | 0.0316 | ThrIGU | C | Methylation | 24170.34 | 4.2 |
| 51 | 24446.29 | 0.0595 | ThrIGU | CC | | 24446.36 | 2.8 |
| 52 | 23608.16 | 0.0452 | TrpBCA | C | | 23608.18 | 0.8 |
| 53 | 23913.15 | 0.1017 | TrpBCA | CC | | 23913.22 | 2.8 |
| 54 | 24428.37 | 0.1243 | TrpBCA | Amino-Acid | | 24428.46 | 3.6 |
| 55 | 25005.54 | 0.2402 | TyrGPA | CC | | 25005.50 | 1.5 |
| 56 | 25334.60 | 0.0278 | TyrGPA | CCA | | 25334.56 | 1.9 |
| 57 | 24700.49 | 0.0822 | TyrGPA | C | | 24700.46 | 0.9 |
| 58 | 24591.40 | 0.0634 | Val&AC | CC | | 24591.36 | 1.5 |
| 59 | 24286.33 | 0.0546 | Val&AC | C | | 24286.32 | 0.2 |
| 60 | 24475.17 | 0.0346 | ValCAC | CCA | | 24475.29 | 5.2 |
| 61 | 24460.15 | 0.8521 | ValIAC | CC | | 24460.24 | 3.7 |
| 62 | 24474.18 | 0.0932 | ValIAC | CC | Methylation | 24474.25 | 3.1 |
| 63 | 24789.24 | 0.0296 | ValIAC | CCA | | 24789.30 | 2.5 |
| 64 | 24155.27 | 0.2068 | ValIAC | C | | 24155.20 | 2.9 |
| 65 | 23242.03 | 0.0804 | | | | | |
| 66 | 23366.10 | 0.0008 | | | | | |
| 67 | 23441.14 | 0.0022 | | | | | |
| 68 | 23445.10 | 0.0015 | | | | | |
| 69 | 23544.12 | 0.0001 | | | | | |
| 70 | 23545.13 | 0.0000 | | | | | |
| 71 | 23546.05 | 0.3990 | | | | | |
| 72 | 23568.12 | 0.0143 | | | | | |
| 73 | 23657.13 | 0.1011 | | | | | |
| 74 | 23667.10 | 0.0273 | | | | | |
| 75 | 23671.16 | 0.6839 | | | | | |
| 76 | 23686.14 | 0.0274 | | | | | |
| 77 | 23687.13 | 0.0240 | | | | | |
| 78 | 23715.12 | 0.0031 | | | | | |
| 79 | 23717.14 | 0.0093 | | | | | |
| 80 | 23724.09 | 0.0037 | | | | | |
| 81 | 23732.15 | 0.0750 | | | | | |
| 82 | 23750.18 | 0.0357 | | | | | |
| 83 | 23791.25 | 0.0116 | | | | | |
| 84 | 23862.18 | 0.0402 | | | | | |
| 85 | 23875.21 | 0.0973 | | | | | |
| 86 | 23895.21 | 0.0171 | | | | | |
| 87 | 23925.26 | 0.0105 | | | | | |
| 88 | 23962.00 | 0.0190 | | | | | |
| 89 | 23976.20 | 0.0376 | | | | | |
| 90 | 23981.22 | 0.0335 | | | | | |
| 91 | 23990.20 | 0.0198 | | | | | |
| 92 | 24041.28 | 0.0204 | | | | | |
| 93 | 24055.27 | 0.0250 | | | | | |
| 94 | 24060.28 | 0.0440 | | | | | |
| 95 | 24074.30 | 0.0707 | | | | | |
| 96 | 24096.31 | 0.1064 | | | | | |
| 97 | 24117.32 | 0.0257 | | | | | |
| 98 | 24130.34 | 0.0035 | | | | | |
| 99 | 24145.29 | 0.0150 | | | | | |
| 100 | 24232.12 | 0.0113 | | | | | |
| 101 | 24346.28 | 0.0171 | | | | | |
| 102 | 24519.22 | 0.0154 | | | | | |
| 103 | 24704.43 | 0.0956 | | | | | |
| 104 | 24720.48 | 0.0002 | | | | | |
| 105 | 24822.46 | 0.0119 | | | | | |
| 106 | 24922.54 | 0.0004 | | | | | |
| 107 | 24989.58 | 0.0023 | | | | | |
| 108 | 25004.45 | 0.0443 | | | | | |
| 109 | 25026.53 | 0.0063 | | | | | |
| 110 | 25058.47 | 0.0010 | | | | | |
| 111 | 25394.53 | 0.0014 | | | | | |
| 112 | 27041.64 | 0.0017 | | | | | |
| 113 | 27211.56 | 0.0143 | | | | | |
| 114 | 27290.67 | 0.0224 | | | | | |
| 115 | 27305.78 | 0.0157 | | | | | |
| 116 | 27313.74 | 0.0005 | | | | | |
| 117 | 27346.73 | 0.0525 | | | | | |
| 118 | 27349.76 | 0.0479 | | | | | |
| 119 | 27364.74 | 0.0009 | | | | | |
| 120 | 27386.66 | 0.0190 | | | | | |
| 121 | 27678.81 | 0.0070 | | | | | |
| 122 | 27717.84 | 0.0071 | | | | | |
| 123 | 27731.83 | 0.0138 | | | | | |
| 124 | 28022.83 | 0.0384 | | | | | |
| 125 | 28036.88 | 0.0841 | | | | | |
| 126 | 28351.86 | 0.0027 | | | | | |
| 127 | 28365.91 | 0.0067 | | | | | |
| 128 | 30818.25 | 0.0001 | | | | | |
| 129 | 30819.24 | 0.0000 | | | | | |

TABLE S3
Verified positions of tRNA nucleotides and modifications in a yeast total tRNA
sample. This table details verified nucleotide and modification positions in yeast total tRNA,
supported by 5′ and 3′ ladder fragments from LC-MS data of controlled acid-hydrolyzed tRNA.
Due to the extensive dataset, individual identities of these nucleotides or modifications are
separately illustrated in FIG. 6h and FIG. S7. To identify fragments, four theoretical mass sets of
ladder fragments were generated for each corresponding tRNA: a 5′ ladder, a 3′ ladder with a
complete CCA end, a 3′ ladder with a truncated CC terminus (lacking ‘A’), and a 3′ ladder with a
sole C end (missing ‘CA’). These sets are based on tRNA sequences from the tRNAdb 2009
database and consider three isoform variations at the 3′ ends. Observed monoisotopic masses
were then cross-referenced with the theoretical fragment masses. A match was confirmed if the
mass disparity between observed and theoretical mases was under 10 ppm, and these
corresponding nucleotide positions are recorded in the table.
To streamline the calculation of ladder fragments for each tRNA position and avoid confusion.

| | tRNA | Positions of Matched 5′ Ladder Fragments |
|---|---|---|
| 1 | AlaIGC | [2, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, |
| | | 36, 37, 38, 39, 41, 42, 49, 52, 54, 55, 56] |
| 2 | Arg1CU | [2, 4, 5, 7, 10, 11, 12, 13, 14, 15, 16, 17, 18, 19, 20, 21, 24, 26, 27, 28, 29, 30, 31, 32, 34, 35, 37, 38, 39, 40, 41, 43, |
| | | 44, 60, 72, 75] |
| 3 | Arg1CU | [2, 6, 8, 9, 11, 18, 23, 24, 29, 31, 32, 34, 37, 38, 39, 40, 41, 44, 62, 74] |
| 4 | ArgICG | [4, 5, 6, 7, 8, 9, 10, 11, 12, 13, 14, 15, 16, 17, 18, 19, 20, 21, 22, 24, 27, 28, 29, 30, 31, 33, 35, 36, 41, 46, 47, 50, |
| | | 54, 61, 62, 71] |
| 5 | AsnGUU | [5, 9, 10, 11, 12, 13, 14, 15, 16, 17, 18, 19, 23, 24, 26, 28, 29, 31, 33, 34, 35, 37, 39, 40, 41, 42, 43, 44, 45, 47, 57, |
| | | 58, 63, 66, 73, 76] |
| 6 | AspGUC | [4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, |
| | | 36, 37, 38, 39, 40, 41, 42, 43, 44, 45, 46, 47, 48, 49, 50, 51, 53, 54, 55, 56, 57, 59, 60, 61, 62] |
| 7 | CysGCA | [2, 4, 5, 6, 7, 8, 9, 10, 11, 12, 13, 14, 15, 16, 17, 18, 19, 21, 22, 23, 24, 25, 26, 27, 32, 33, 34, 35, 36, 37, 39, 45, 46, |
| | | 47, 53, 55, 56, 58, 68] |
| 8 | Glu3UC | [4, 5, 6, 7, 8, 9, 10, 11, 12, 13, 14, 15, 16, 17, 18, 19, 20, 21, 22, 23, 24, 25, 26, 27, 28, 29, 30, 32, 33, 35, 36, 37, |
| | | 53, 75] |
| 9 | Gly.CC | [2, 4, 5, 7, 8, 11, 12, 19, 22, 39, 41, 49, 50, 52, 72] |
| 10 | GlyGCC | [2, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, |
| | | 37, 38, 39, 40, 41, 42, 43, 44, 45, 46, 47, 50, 52] |
| 11 | HisGUG | [2, 4, 5, 6, 7, 8, 9, 10, 11, 12, 13, 14, 15, 16, 17, 19, 20, 21, 22, 23, 24, 27, 28, 29, 30, 31, 34, 35, 36, 38, 42, 44, 46, |
| | | 48, 53, 54, 71, 74, 76] |
| 12 | HisGUG | [2, 4, 5, 6, 7, 8, 9, 10, 11, 12, 13, 14, 15, 16, 17, 19, 20, 21, 22, 23, 24, 27, 28,29, 30, 31, 32, 34, 35, 36, 38, 46, 47, |
| | | 49, 51, 54] |
| 13 | IleIAU | [2, 3, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, |
| | | 36, 37, 41, 42, 47, 48, 49, 53, 55, 56, 60, 61, 72] |
| 14 | IniCAU | [4, 7, 9, 10, 11, 12, 13, 15, 16, 17, 18, 19, 20, 21, 22, 24, 29, 30, 31, 32, 34, 35, 41, 49, 53, 64, 72] |
| 15 | Leu?AA | [2, 3, 4, 5, 6, 9, 10, 11, 12, 13, 14, 15, 16, 17, 18, 19, 20, 21, 24, 28, 29, 31, 33, 34, 35, 39, 41, 44, 56, 65, 74] |
| 16 | LeuUAA | [2, 8, 9, 10, 11, 12, 13, 16, 17, 18, 19, 21, 22, 23, 24, 25, 26, 28, 29, 30, 31, 37, 45, 48, 55, 72, 73, 75, 83] |
| 17 | LeuUAG | [2, 7, 8, 9, 10, 11, 12, 13, 14, 16, 18, 19, 20, 21, 22, 24, 25, 26, 27, 28, 29, 32, 33, 34, 35, 36, 37, 42, 43, 47, 52, |
| | | 73] |
| 18 | Lys)UU | [4, 5, 6, 7, 8, 9, 10, 11, 12, 13, 14, 15, 16, 17, 18, 19, 20, 21, 22, 23, 24, 25, 26, 27, 28, 29, 30, 32, 33, 42, 43, 44, |
| | | 47, 48, 49, 65, 67] |
| 19 | LysCUU | [2, 4, 5, 6, 7, 8, 9, 10, 11, 12, 13, 14, 15, 16, 17, 18, 19, 20, 21, 23, 24, 27, 29, 33, 35, 36, 38, 42, 49, 51, 56, 57, 60, |
| | | 66, 74, 76] |
| 20 | MetCAU | [2, 5, 6, 7, 8, 9, 10, 11, 12, 13, 14, 15, 16, 18, 19, 20, 21, 23, 24, 26, 27, 28, 29, 30, 32, 33, 37, 39, 41, 47, 48, 54, |
| | | 65, 72, 73] |
| 21 | Phe#AA | [2, 4, 5, 6, 7, 8, 9, 10, 11, 12, 13, 14, 15, 16, 17, 18, 19, 20, 21, 22, 24, 25, 28, 30, 32, 34, 35, 37, 38, 55, 56, 58, |
| | | 74] |
| 22 | Phe#AA | [2, 4, 5, 6, 9, 10, 16, 17, 18, 19, 21, 24, 26, 27, 28, 29, 30, 31, 32, 34, 35, 38, 40, 43, 55, 56, 58] |
| 23 | ProNGG | [2, 5, 6, 7, 8, 9, 10, 11, 12, 13, 14, 15, 16, 17, 18, 19, 20, 21, 22, 23, 24, 25, 26, 28, 44, 57, 59] |
| 24 | ProNGG | [2, 5, 6, 7, 8, 9, 10, 16, 22, 28, 30, 31, 32, 45, 50, 74] |
| 25 | SerIGA | [2, 4, 5, 6, 7, 8, 9, 10, 11, 12, 13, 14, 15, 16, 18, 20, 22, 25, 27, 28, 29, 32, 34, 41, 42, 45, 55, 59, 65, 75] |
| 26 | SerIGA | [2, 4, 5, 6, 7, 8, 9, 10, 11, 12, 13, 14, 15, 16, 18, 20, 22, 25, 27, 28, 29, 32, 34, 41, 42, 45, 54, 57, 64, 65, 66, 68, 79, |
| | | 83, 84] |
| 27 | SerNGA | [2, 4, 6, 7, 8, 9, 10, 11, 12, 13, 14, 15, 16, 18, 20, 22, 25, 27, 29, 30, 34, 38, 41, 43, 45, 54, 57, 58, 71, 74] |
| 28 | ThrIGU | [2, 5, 6, 7, 8, 9, 10, 11, 12, 13, 14, 15, 16, 17, 18, 19, 20, 21, 22, 29, 30, 33, 34, 36, 39, 40, 42, 43, 47, 52, 53, 54, |
| | | 57, 59, 60] |
| 29 | ThrIGU | [2, 5, 6, 7, 8, 9, 10, 11, 12, 13, 14, 15, 16, 17, 18, 19, 20, 21, 22, 29, 30, 33, 34, 36, 39, 40, 42, 43, 47, 61, 73] |
| 30 | TrpBCA | [5, 6, 8, 9, 10, 11, 12, 13, 14, 15, 16, 17, 18, 19, 22, 23, 24, 25, 27, 32, 33, 37, 43, 44, 52, 56, 72] |
| 31 | TyrGPA | [4, 9, 10, 11, 12, 13, 14, 15, 17, 18, 19, 20, 21, 22, 23, 24, 25, 26, 28, 30, 34, 35, 37, 40, 44, 47, 50, 52, 60, 72] |
| 32 | Val&AC | [2, 3, 5, 6, 7, 8, 9, 10, 11, 16, 17, 19, 20, 21, 22, 23, 24, 26, 29, 31, 37, 44, 48, 53, 55, 56, 57, 58, 73] |
| 33 | ValCAC | [5, 6, 7, 8, 9, 10, 12, 15, 16, 17, 18, 20, 26, 29, 30, 33, 34, 40, 41, 47, 50, 54] |
| 34 | ValIAC | [2, 3, 4, 5, 7, 8, 9, 10, 11, 12, 13, 14, 15, 16, 17, 18, 21, 22, 23, 24, 25, 26, 27, 28, 29, 30, 31, 32, 33, 34, 35, 36, 37, |
| | | 38, 39, 40, 41, 42, 43, 45, 46, 47, 49, 50, 51, 52, 53, 55, 60] |
| | tRNA | Positions of Matched 3′ Ladder Fragments |
| 1 | AlaIGC | [75, 74, 73, 72, 70, 69, 68, 67, 66, 65, 64, 63, 62, 61, 60, 59, 58, 57, 56, 55, 54, 53, 51, 50, 49, 48, 45, 44, 43, 40, |
| | | 38, 35, 27, 20, 15, 3] |
| 2 | Arg1CU | [74, 73, 70, 68, 67, 66, 65, 64, 63, 62, 61, 60, 59, 58, 56, 55, 52, 50, 49, 48, 46, 45, 44, 43, 41, 39, 38, 37, 31, 27, |
| | | 16, 12, 3, 2] |
| 3 | Arg1CU | [74, 73, 72, 71, 70, 69, 68, 67, 66, 65, 64, 60, 59, 58, 56, 55, 52, 49, 46, 42, 40, 38, 36, 35, 33, 32, 28, 21, 20, 3] |
| 4 | ArgICG | [75, 74, 72, 70, 69, 62, 61, 60, 59, 58, 57, 56, 55, 54, 53, 52, 49, 48, 47, 46, 44, 40, 38, 35, 33, 29, 23, 22, 16, 15, |
| | | 21 |
| 5 | AsnGUU | [76, 75, 73, 72, 71, 70, 68, 67, 66, 65, 64, 63, 62, 61, 60, 59, 58, 57, 54, 51, 50, 49, 47, 45, 44, 43, 40, 37, 25, 23, |
| | | 51 |
| 6 | AspGUC | [74, 73, 71, 69, 68, 65, 64, 63, 62, 61, 60, 59, 58, 57, 56, 55, 54, 53, 52, 51, 49, 48, 47, 46, 45, 44, 43, 42, 41, 39, |
| | | 38, 37, 36, 35, 32, 31, 30, 29, 28, 26, 25, 24, 22, 16, 15, 14, 10, 2] |
| 7 | CysGCA | [74, 73, 71, 70, 69, 68, 65, 64, 63, 62, 60, 59, 57, 55, 54, 53, 52, 51, 47, 46, 45, |
| | | 44, 41, 34, 32, 27, 26, 24, 22, 21, 16, 6] |
| 8 | Glu3UC | [74, 73, 71, 69, 68, 67, 66, 65, 64, 63, 62, 61, 60, 59, 58, 57, 56, 55, 54, 53, 52, 51, 49, 48, 47, 46, 45, 44, 43, 42, |
| | | 40, 39, 38, 37, 36, 34, 30, 29, 27, 26, 25, 24, 22, 17, 15, 8] |
| 9 | Gly.CC | [74, 73, 72, 71, 70, 69, 68, 67, 65, 64, 63, 62, 61, 60, 59, 58, 57, 56, 55, 54, 53, 52, 50, 49, 48, 47, 39, 38, 34, 33, |
| | | 31, 21, 20] |
| 10 | GlyGCC | [72, 71, 70, 69, 68, 64, 63, 62, 60, 59, 58, 57, 56, 55, 53, 51, 50, 49, 48, 45, 44, 43, 42, 41, 40, 38, 35, 33, 32, 31, |
| | | 29, 26, 25, 24, 23, 22, 21, 14] |
| 11 | HisGUG | [75, 74, 73, 72, 71, 70, 69, 68, 67, 66, 65, 60, 59, 58, 57, 56, 54, 53, 51, 50, 48, 47, 46, 45, 44, 41, 40, 39, 38, 35, |
| | | 32, 29, 28, 27, 24, 21, 13] |
| 12 | HisGUG | [75, 74, 73, 72, 71, 70, 69, 68, 67, 66, 65, 64, 63, 62, 61, 60, 59, 58, 57, 56, 55, 54, 53, 52, 49, 48, 47, 46, 45, 44, |
| | | 43, 41, 39, 38, 37, 36, 35, 34, 31, 30, 29, 28, 27, 26, 25, 22, 18, 14, 11] |
| 13 | IleIAU | [76, 75, 74, 73, 70, 68, 66, 61, 60, 59, 58, 57, 56, 55, 54, 53, 52, 51, 50, 49, 48, 47, 45, 41, 36, 35, 32, 28, 25, 24, |
| | | 17, 5, 4] |
| 14 | IniCAU | [74, 73, 72, 70, 69, 68, 64, 63, 62, 61, 60, 59, 58, 57, 56, 55, 54, 53, 52, 51, 49, 48, 47, 46, 45, 44, 43, 42, 41, 40, |
| | | 39, 35, 34, 33, 30, 28, 4] |
| 15 | Leu?AA | [84, 83, 82, 81, 75, 74, 73, 72, 71, 70, 69, 68, 67, 66, 65, 63, 62, 61, 60, 59, 57, 55, 53, 52, 50, 47, 44, 43, 42, 41, |
| | | 38, 37, 34, 27] |
| 16 | LeuUAA | [86, 85, 84, 83, 82, 80, 78, 77, 76, 73, 72, 71, 70, 69, 67, 64, 63, 62, 61, 60, 57, 56, 49, 46, 45, 44, 41, 40, 38, 35, |
| | | 34, 33, 31, 28, 14] |
| 17 | LeuUAG | [84, 83, 82, 81, 80, 79, 78, 76, 74, 73, 72, 71, 70, 69, 68, 65, 62, 60, 59, 56, 55, 54, 51, 50, 48, 47, 45, 44, 42, 37, |
| | | 36, 35, 25, 23, 11, 10, 8] |
| 18 | LysCUU | [75, 74, 72, 70, 69, 67, 65, 64, 63, 62, 61, 60, 59, 54, 53, 52, 51, 50, 49, 48, 47, 44, 43, 41, 37, 29, 28, 26, 25, 22, |
| | | 18, 17, 16, 14] |
| 19 | LysCUU | [75, 74, 72, 71, 70, 69, 68, 65, 64, 63, 62, 61, 60, 59, 58, 56, 55, 54, 52, 51, 50, 49, 48, 47, 46, 45, 44, 42, 39, 38, |
| | | 15, 5] |
| 20 | MetCAU | [75, 74, 73, 72, 71, 70, 69, 68, 67, 64, 63, 61, 60, 59, 57, 56, 53, 52, 51, 50, 49, 48, 46, 45, 44, 39, 38, 35, 34, 33, |
| | | 32, 28, 23, 22, 21, 19, 4] |
| 21 | Phe#AA | [75, 74, 73, 72, 71, 69, 68, 67, 65, 64, 63, 62, 61, 60, 59, 58, 56, 55, 54, 52, 51, 49, 47, 45, 44, 42, 40, 39, 37, 35, |
| | | 33, 32, 30, 29, 28, 27, 25, 24, 20, 10] |
| 22 | Phe#AA | [75, 74, 73, 72, 71, 69, 68, 67, 66, 65, 64, 63, 62, 61, 60, 59, 58, 57, 55, 54, 53, 52, 51, 50, 49, 48, 47, 46, 43, 42, |
| | | 41, 38, 30, 29, 28, 19, 12] |
| 23 | ProNGG | [74, 73, 72, 65, 64, 62, 61, 60, 58, 57, 53, 52, 51, 50, 48, 47, 46, 41, 35, 34, 30, 29, 27, 26, 25, 2] |
| 24 | ProNGG | [74, 73, 72, 65, 64, 62, 61, 60, 58, 57, 53, 52, 51, 50, 48, 47, 46, 41, 35, 34, 30, 29, 27, 26, 25, 6] |
| 25 | SerIGA | [84, 83, 81, 80, 79, 76, 75, 74, 73, 72, 71, 70, 69, 68, 67, 66, 59, 58, 57, 55, 50, 48, 42, 40, 38, 37, 35, 30, 10] |
| 26 | SerIGA | [84, 83, 81, 80, 79, 76, 75, 74, 73, 72, 71, 70, 69, 66, 65, 61, 59, 55, 53, 52, 48, 41, 39, 32, 29, 22] |
| 27 | SerNGA | [84, 83, 81, 80, 79, 77, 76, 75, 74, 73, 72, 71, 70, 69, 64, 63, 58, 51, 50, 46, 44, 43, 40, 33, 26, 19, 13, 11] |
| 28 | ThrIGU | [75, 74, 73, 72, 71, 70, 68, 66, 65, 64, 63, 62, 61, 60, 59, 58, 56, 53, 52, 51, 50, 49, 48, 47, 46, 45, 43, 42, 41, 40, |
| | | 39, 34, 29, 28, 21, 18] |
| 29 | ThrIGU | [75, 74, 73, 72, 71, 70, 68, 66, 65, 64, 63, 61, 60, 59, 56, 55, 54, 53, 52, 51, 50, 49, 48, 45, 44, 43, 39, 33, 30, 28, |
| | | 25, 23, 17, 10, 3] |
| 30 | TrpBCA | [74, 73, 72, 71, 70, 68, 65, 64, 62, 61, 60, 57, 56, 54, 53, 52, 51, 49, 48, 47, 44, 43, 40, 38, 37, 35, 33, 32, 31, 29, |
| | | 28, 27, 25, 23, 22, 16, 8] |
| 31 | TyrGPA | [77, 76, 75, 74, 72, 71, 66, 65, 57, 53, 51, 49, 48, 47, 46, 44, 42, 37, 36, 34, 32, 30, 14, 10, 9] |
| 32 | Val&AC | [76, 75, 74, 73, 72, 71, 69, 68, 66, 65, 64, 63, 62, 61, 60, 58, 57, 54, 53, 52, 50, 48, 47, 46, 45, 44, 42, 41, 40, 39, |
| | | 32, 29, 26, 23, 15] |
| 33 | ValCAC | [75, 74, 73, 72, 68, 67, 65, 64, 63, 61, 60, 59, 57, 56, 53, 52, 51, 49, 47, 43, 37, 36, 35, 34, 32, 31, 29, 27, 26, 23, |
| | | 21, 16, 13, 4] |
| 34 | ValIAC | [76, 75, 74, 73, 72, 71, 70, 69, 68, 67, 66, 65, 64, 63, 62, 61, 60, 59, 58, 57, 56, 55, 54, 53, 52, 51, 50, 49, 46, 45, |
| | | 44, 43, 40, 39, 37, 31, 29, 28, 26, 24, 23, 22, 20, 19, 15, 4, 2] |
| | tRNA | Positions of Matched 3′ Ladder Fragments with a CC End |
| 1 | AlaIGC | [73, 72, 70, 69, 68, 67, 65, 64, 63, 62, 61, 60, 59, 58, 57, 56, 55, 54, 53, 52, 51, 50, 49, 48, 47, 46, 45, 44, 43, 42, 41, |
| | | 40, 38, 37, 36, 35, 32, 31, 30, 28, 27, 23, 20, 19, 17, 16, 14, 9] |
| 2 | Arg1CU | [69, 68, 67, 66, 65, 64, 63, 62, 61, 60, 59, 58, 56, 55, 54, 53, 52, 51, 50, 49, 48, 47, 46, 45, 43, 42, 40, 39, 38, 37, 36, |
| | | 30, 29, 17, 15, 5, 4] |
| 3 | Arg1CU | [69, 68, 66, 65, 64, 63, 62, 61, 60, 59, 58, 56, 55, 53, 49, 48, 47, 45, 41, 37, 35, 33, 31, 20, 19] |
| 4 | ArgICG | [71, 69, 68, 67, 65, 64, 63, 62, 61, 60, 59, 58, 57, 56, 55, 54, 53, 52, 51, 50, 49, 48, 47, 46, 43, 42, 40, 39, 37, 35, 32, |
| | | 31, 29, 21, 16, 15, 14] |
| 5 | AsnGUU | [71, 70, 69, 68, 67, 66, 65, 64, 63, 62, 61, 60, 59, 58, 57, 56, 55, 54, 53, 50, 49, 48, 46, 44, 42, 39, 36, 35, 34, 33, 31, |
| | | 30, 29, 22, 19, 18] |
| 6 | AspGUC | [69, 67, 66, 64, 63, 62, 61, 60, 59, 58, 57, 56, 55, 54, 53, 52, 51, 50, 49, 48, 47, 46, 45, 44, 43, 42, 41, 40, 39, 38, 37, |
| | | 36, 35, 34, 33, 32, 31, 30, 29, 28, 26, 25, 24, 23, 22, 21, 20, 19, 18, 16, 15, 14, 13, 11, 10, 9] |
| 7 | CysGCA | [69, 68, 67, 65, 64, 63, 62, 61, 60, 59, 58, 57, 56, 55, 54, 53, 52, 51, 50, 49, 45, 33, 27, 26, 25, 23, 22, 20, 19, 18, 16] |
| 8 | Glu3UC | [69, 67, 66, 65, 64, 63, 61, 60, 59, 58, 57, 56, 55, 54, 53, 52, 51, 50, 49, 48, 47, 46, 45, 44, 43, 42, 40, 38, 37, 36, 35, |
| | | 34, 33, 32, 31, 30, 29, 28, 26, 25, 24, 23, 22, 21, 20, 19, 16, 14, 12, 7] |
| 9 | Gly.CC | [72, 71, 70, 69, 68, 67, 66, 64, 63, 62, 61, 60, 59, 58, 57, 56, 55, 54, 53, 52, 51, 49, 48, 47, 46, 45, 39, 38, 37, 35, 31, |
| | | 30] |
| 10 | GlyGCC | [70, 69, 68, 65, 64, 63, 62, 61, 60, 59, 58, 57, 56, 55, 54, 53, 52, 51, 50, 49, 48, 47, 45, 43, 42, 37, 33, 29, 28, 23, 22, |
| | | 21, 20, 17] |
| 11 | HisGUG | [73, 72, 71, 69, 68, 66, 64, 62, 60, 59, 58, 57, 51, 50, 47, 46, 45, 43, 39, 38, 37, 35, 27, 26, 24, 20, 19, 3] |
| 12 | HisGUG | [73, 72, 71, 69, 68, 67, 66, 65, 64, 63, 62, 61, 60, 59, 58, 57, 56, 55, 54, 53, 52, 51, 50, 49, 47, 46, 45, 44, 42, 41, 38, |
| | | 37, 36, 35, 33, 32, 31, 29, 27, 26, 25, 24, 20, 13, 10] |
| 13 | IleIAU | [74, 73, 69, 67, 66, 64, 63, 62, 61, 60, 59, 58, 57, 56, 55, 54, 53, 52, 51, 50, 49, 48, 47, 46, 45, 44, 43, 42, 41, 38, 35, |
| | | 34, 33, 29, 28, 27, 23, 21, 20, 19, 18, 9, 7, 6] |
| 14 | IniCAU | [72, 70, 69, 68, 67, 64, 63, 62, 61, 59, 58, 55, 54, 53, 51, 50, 48, 47, 46, 45, 43, 42, 41, 40, 39, 34, 32, 31, 28, 27, 26, |
| | | 25, 3] |
| 15 | Leu?AA | [82, 81, 77, 76, 74, 73, 72, 71, 70, 69, 68, 67, 66, 65, 64, 63, 62, 61, 60, 59, 58, 57, 56, 55, 54, 53, 52, 51, 50, 49, 48, |
| | | 46, 45, 44, 43, 42, 41, 38, 37, 36, 35, 31, 29, 28, 26, 25, 8, 7, 4, 3] |
| 16 | LeuUAA | [84, 83, 82, 79, 78, 77, 76, 75, 73, 72, 71, 70, 69, 68, 65, 63, 62, 59, 58, 56, 55, 53, 50, 49, 48, 45, 43, 42, 41, 39, 38, |
| | | 37, 35, 32, 30, 24, 13] |
| 17 | LeuUAG | [82, 81, 80, 79, 77, 76, 75, 74, 73, 72, 71, 70, 69, 68, 67, 66, 65, 64, 63, 61, 60, 59, 58, 57, 56, 55, 54, 52, 51, 50, 49, |
| | | 48, 47, 46, 45, 44, 43, 41, 38, 37, 36, 34, 22] |
| 18 | Lys)UU | [70, 69, 68, 67, 66, 65, 64, 63, 62, 61, 60, 59, 58, 55, 51, 50, 49, 48, 47, 46, 43, 42, 41, 39, 31, 28, 26, 25, 21, 20, 18, |
| | | 17, 2] |
| 19 | LysCUU | [70, 69, 68, 65, 64, 63, 62, 61, 60, 59, 57, 56, 51, 50, 49, 48, 47, 46, 45, 44, 43, 42, 41, 40, 38, 33, 30, 26, 14, 6] |
| 20 | MetCAU | [73, 72, 71, 70, 69, 68, 67, 64, 63, 62, 61, 60, 59, 58, 56, 52, 50, 49, 47, 45, 44, 43, 41, 40, 38, 32, 30, 28, 26, 25, |
| | | 22, 21, 20, 15, 3] |
| 21 | Phe#AA | [73, 72, 71, 69, 67, 66, 65, 64, 63, 62, 61, 60, 59, 58, 57, 56, 55, 54, 53, 52, 51, 50, 49, 48, 47, 46, 45, 44, 42, 41, |
| | | 40, 39, 38, 37, 36, 34, 31, 29, 28, 23, 18, 9] |
| 22 | Phe#AA | [73, 72, 71, 69, 67, 66, 65, 64, 63, 62, 58, 57, 56, 55, 54, 53, 50, 49, 47, 45, 43, 42, 41, 37, 29, 17] |
| 23 | ProNGG | [70, 68, 66, 65, 64, 63, 62, 61, 59, 58, 57, 56, 55, 54, 53, 52, 49, 48, 47, 46, 42, 36, 35, 34, 32, 30, 29, 28, 27, 24, |
| | | 18] |
| 24 | ProNGG | [70, 68, 66, 65, 64, 63, 62, 61, 59, 58, 57, 56, 55, 54, 53, 52, 49, 48, 47, 46, 42, 36, 35, 34, 32, 30, 29, 28, 27, 24, |
| | | 18] |
| 25 | SerIGA | [79, 78, 77, 76, 75, 74, 73, 72, 71, 70, 69, 68, 67, 66, 65, 63, 62, 58, 55, 54, 50, 49, 48, 47, 44, 41, 36, 32, 29] |
| 26 | SerIGA | [79, 78, 77, 76, 75, 74, 73, 72, 71, 70, 69, 68, 66, 65, 64, 62, 61, 60, 58, 56, 54, 53, 52, 51, 50, 49, 48, 47, 45, 44, |
| | | 43, 42, 41, 38, 37, 35, 31, 28, 21, 12, 8, 7] |
| 27 | SerNGA | [79, 78, 77, 76, 75, 73, 72, 71, 70, 69, 68, 59, 58, 57, 55, 54, 53, 51, 50, 49, 48, 47, 46, 45, 41, 40, 39, 32, 28, 26, |
| | | 11] |
| 28 | ThrIGU | [73, 72, 71, 70, 69, 68, 67, 66, 65, 64, 63, 62, 61, 60, 59, 58, 57, 56, 55, 54, 53, 52, 51, 50, 49, 48, 47, 46, 45, 44, |
| | | 43, 42, 41, 40, 38, 37, 35, 33, 32, 30, 28, 24, 23, 20, 19, 18, 17] |
| 29 | ThrIGU | [73, 72, 71, 70, 69, 68, 67, 66, 64, 61, 60, 59, 58, 51, 50, 49, 47, 45, 44, 43, 42, 41, 40, 39, 38, 35, 30, 29, 27, 26, |
| | | 25, 22] |
| 30 | TrpBCA | [72, 71, 70, 68, 67, 65, 64, 63, 62, 61, 60, 59, 58, 57, 56, 55, 54, 53, 52, 51, 50, 48, 47, 46, 45, 43, 42, 40, 39, 37, |
| | | 36, 31, 30, 27, 22, 21, 16, 15] |
| 31 | TyrGPA | [75, 73, 71, 70, 64, 63, 62, 61, 60, 59, 58, 57, 55, 53, 52, 51, 50, 49, 48, 47, 46, 45, 44, 43, 42, 39, 33, 31, 25, 16, |
| | | 13, 12, 9] |
| 32 | Val&AC | [74, 73, 72, 71, 70, 69, 68, 67, 66, 65, 64, 63, 62, 61, 60, 59, 58, 57, 56, 55, 54, 53, 52, 51, 50, 48, 47, 46, 45, 44, |
| | | 43, 42, 40, 39, 38, 35, 34, 30, 29, 28, 26, 25, 22, 18, 17, 15, 14] |
| 33 | ValCAC | [73, 72, 71, 69, 68, 67, 66, 65, 64, 63, 62, 61, 60, 59, 58, 57, 56, 55, 54, 53, 52, 51, 50, 49, 47, 46, 45, 42, 41, 40, |
| | | 35, 29, 13] |
| 34 | ValIAC | [74, 73, 72, 71, 70, 69, 68, 67, 66, 65, 63, 62, 61, 60, 59, 58, 57, 56, 55, 54, 53, 52, 51, 50, 49, 48, 47, 46, 45, 44, |
| | | 42, 41, 40, 39, 38, 37, 36, 35, 34, 33, 32, 31, 30, 29, 28, 27, 26, 25, 24, 23, 22, 21, 20, 19, 16, 15, 14, 13, 12, 5] |
| | tRNA | Positions of Matched 3′ Ladder Fragments with a C End |
| 1 | AlaIGC | [73, 72, 71, 70, 69, 68, 66, 65, 64, 63, 62, 61, 60, 59, 58, 57, 56, 54, 53, 52, 51, 50, 49, 48, 46, 44, 43, 42, 38, 37, 36, |
| | | 32, 31, 30, 29, 28, 27, 26, 22, 2] |
| 2 | Arg1CU | [72, 66, 65, 64, 63, 62, 61, 60, 59, 58, 57, 56, 55, 54, 53, 52, 50, 48, 46, 45, 44, 43, 42, 41, 40, 39, 37, 36, 26, 18, 5, |
| | | 4] |
| 3 | Arg1CU | [68, 67, 66, 65, 64, 63, 62, 61, 60, 59, 58, 55, 54, 52, 51, 50, 47, 46, 45, 44, 43, 41, 38, 35, 34, 30, 29, 26, 19] |
| 4 | ArgICG | [73, 68, 66, 65, 64, 63, 62, 61, 60, 59, 58, 57, 56, 55, 54, 52, 51, 50, 49, 48, 46, 45, 44, 43, 39, 38, 37, 36, 31, 29, 28, |
| | | 27, 23, 21, 20, 14, 13] |
| 5 | AsnGUU | [74, 67, 66, 65, 64, 63, 62, 61, 60, 58, 57, 56, 55, 54, 53, 52, 50, 49, 44, 43, 41, 39, 34, 33, 30, 23, 20] |
| 6 | AspGUC | [72, 68, 67, 66, 64, 63, 62, 61, 60, 59, 58, 57, 56, 55, 53, 52, 51, 50, 49, 48, 47, 46, 45, 44, 43, 42, 41, 39, 38, 37, 36, |
| | | 35, 34, 32, 31, 30, 29, 28, 27, 26, 24, 23, 22, 20, 19, 18, 17, 15, 12, 10] |
| 7 | CysGCA | [68, 67, 66, 65, 64, 63, 62, 61, 60, 59, 58, 57, 56, 55, 54, 53, 51, 50, 49, 48, 47, 46, 45, 44, 43, 42, 40, 38, 36, 35, 28, |
| | | 24, 23, 22, 21, 20, 19, 18, 17] |
| 8 | Glu3UC | [72, 68, 67, 66, 65, 64, 63, 62, 60, 59, 58, 57, 56, 55, 53, 52, 51, 50, 49, 48, 47, 46, 45, 44, 43, 42, 40, 39, 37, 36, 34, |
| | | 32, 30, 28, 27, 26, 25, 24, 23, 22, 20, 19, 17, 15, 14, 13, 7] |
| 9 | Gly.CC | [72, 71, 69, 68, 67, 66, 65, 64, 63, 62, 61, 60, 59, 58, 57, 56, 55, 54, 53, 51, 50, 48, 47, 46, 45, 43, 40, 39, 37, 36, 34, |
| | | 29, 23, 18, 15] |
| 10 | GlyGCC | [70, 69, 67, 65, 63, 62, 61, 60, 59, 58, 57, 56, 55, 54, 53, 52, 51, 50, 49, 48, 47, 46, 39, 33, 32, 22] |
| 11 | HisGUG | [73, 72, 69, 68, 66, 64, 63, 62, 61, 60, 59, 58, 57, 56, 55, 53, 52, 51, 50, 46, 42, 40, 38, 37, 34, 33, 30, 27, 26, 25, 22, |
| | | 16, 15, 13, 12] |
| 12 | HisGUG | [73, 72, 69, 67, 65, 64, 63, 62, 61, 60, 59, 58, 57, 56, 54, 53, 52, 51, 50, 49, 48, 47, 45, 44, 43, 42, 40, 38, 37, 35, 34, |
| | | 33, 31, 29, 28, 27, 26, 25, 19, 15] |
| 13 | IleIAU | [74, 73, 72, 71, 70, 69, 68, 67, 66, 65, 64, 63, 62, 61, 59, 58, 57, 56, 55, 54, 53, 52, 51, 50, 48, 47, 46, 42, 40, 39, 37, |
| | | 36, 33, 28, 26, 24, 23, 21, 6] |
| 14 | IniCAU | [72, 68, 67, 66, 63, 61, 60, 59, 58, 57, 53, 52, 50, 49, 47, 44, 42, 40, 39, 38, 37, 34, 33, 32, 31, 30, 29, 27, 26, 24, 23] |
| 15 | Leu?AA | [82, 81, 80, 79, 76, 74, 72, 71, 70, 69, 68, 67, 66, 65, 63, 62, 61, 60, 59, 58, 57, 55, 54, 53, 52, 51, 49, 48, 45, 44, 41, |
| | | 38, 36, 31, 28, 9, 8, 6, 3] |
| 16 | LeuUAA | [84, 83, 77, 76, 75, 74, 73, 72, 71, 70, 69, 68, 67, 66, 65, 63, 61, 58, 57, 55, 53, 52, 50, 49, 44, 42, 41, 40, 37, 35, 33, |
| | | 32, 30, 29, 28, 25, 2] |
| 17 | LeuUAG | [82, 81, 79, 75, 72, 71, 70, 69, 68, 67, 66, 65, 64, 63, 62, 60, 59, 58, 57, 56, 55, 51, 48, 45, 44, 35, 34, 33, 31, 27, 24] |
| 18 | Lys)UU | [73, 68, 66, 65, 64, 63, 62, 61, 60, 59, 58, 57, 55, 54, 52, 51, 50, 49, 48, 47, 46, 42, 40, 35, 34, 30, 28, 26, 25, 21, 20, |
| | | 14] |
| 19 | LysCUU | [68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, |
| | | 37, 33, 32, 29, 26, 25, 21, 20, 13] |
| 20 | MetCAU | [73, 72, 70, 68, 67, 66, 64, 63, 62, 61, 60, 59, 58, 57, 55, 53, 52, 51, 50, 48, 47, 46, 45, 44, 43, 41, 40, 39, 38, 37, 29, |
| | | 25, 22, 21, 20, 19, 14, 2] |
| 21 | Phe#AA | [73, 72, 70, 69, 65, 63, 62, 61, 60, 59, 58, 57, 56, 54, 53, 52, 51, 48, 47, 46, 45, 42, 39, 29, 28, 27, 23, 17, 16] |
| 22 | Phe#AA | [73, 72, 70, 69, 66, 65, 64, 63, 62, 61, 58, 57, 56, 55, 54, 53, 51, 50, 48, 47, 46, 40, 38, 28, 17] |
| 23 | ProNGG | [69, 68, 67, 66, 65, 64, 63, 62, 61, 60, 59, 58, 57, 56, 55, 54, 53, 52, 51, 50, 49, 48, 47, 46, 45, 33, 31, 30, 29, 27, 25, |
| | | 24, 21, 15, 5] |
| 24 | ProNGG | [69, 68, 67, 66, 65, 64, 63, 62, 61, 60, 59, 58, 57, 56, 55, 54, 53, 52, 51, 50, 49, 48, 47, 46, 45, 33, 31, 30, 29, 27, 25, |
| | | 24, 21, 15] |
| 25 | SerIGA | [82, 75, 74, 73, 72, 71, 70, 68, 67, 66, 65, 61, 60, 54, 53, 52, 51, 48, 47, 43, 42, 41, 40, 35, 30, 23] |
| 26 | SerIGA | [82, 75, 74, 73, 72, 71, 70, 68, 67, 66, 65, 63, 62, 61, 59, 58, 55, 53, 52, 48, 43, 40, 37, 36, 35, 32, 28, 11, 9, 7, 6] |
| 27 | SerNGA | [82, 76, 75, 74, 73, 72, 71, 70, 68, 67, 66, 63, 62, 61, 54, 53, 52, 50, 49, 48, 47, 46, 42, 41, 40, 35, 34, 32, 31, 27, 26, |
| | | 10] |
| 28 | ThrIGU | [73, 72, 70, 68, 67, 64, 63, 62, 61, 59, 58, 57, 56, 54, 53, 52, 51, 48, 47, 46, 45, 43, 38, 31, 27, 23, 22, 21, 20, 19] |
| 29 | ThrIGU | [73, 72, 70, 68, 67, 65, 64, 63, 60, 59, 55, 50, 47, 45, 44, 43, 40, 35, 32, 28, 25, 22, 17, 14, 9] |
| 30 | TrpBCA | [72, 71, 66, 64, 63, 62, 61, 60, 59, 58, 57, 56, 55, 54, 53, 50, 49, 48, 41, 40, 39, 35, 30, 23, 22, 21, 20, 18, 15, 14, 12] |
| 31 | TyrGPA | [75, 70, 69, 63, 62, 61, 60, 58, 57, 56, 55, 54, 52, 51, 50, 49, 48, 47, 46, 43, 42, 36, 31, 22, 12, 11, 4] |
| 32 | Val&AC | [74, 73, 68, 66, 65, 64, 63, 62, 61, 60, 59, 58, 57, 55, 53, 51, 50, 49, 48, 46, 44, 43, 42, 39, 38, 37, 34, 33, 29, 28, 27, |
| | | 23, 21, 13, 3, 2] |
| 33 | ValCAC | [73, 72, 71, 68, 64, 63, 62, 61, 60, 59, 58, 57, 56, 54, 52, 50, 49, 48, 47, 46, 45, 44, 43, 42, 41, 40, 39, 35, 34, 28, 26, |
| | | 24, 21, 15, 13] |
| 34 | ValIAC | [74, 73, 70, 69, 68, 67, 66, 65, 64, 63, 62, 61, 60, 59, 58, 57, 56, 55, 54, 53, 52, 51, 50, 49, 47, 46, 44, 43, 41, 39, 38, |
| | | 37, 35, 32, 31, 30, 29, 27, 26, 23, 13, 12, 11, 5] |

TABLE 4
Site-specific quantitative mapping of partial nucleotide modifications in a yeast 343
total tRNA sample. This table outlines the locations, identities, and stoichiometries of partial
344 modifications identified in various tRNA species within the yeast total tRNA sample.
345 Quantification was limited to partial modifications that were confirmed by at least two
newly 346 branched ladder fragments after the modification site. Modifications are classified
into two 347 categories using a specific criterion: the presence of at least six ladder fragments
stemming from 348 the modification site. Fulfilling this condition assigns the modification to
the first category, 349 reflecting a high level of confidence in its identification. Modifications
in the second category, 350 although having fewer than six branching ladder fragments (BLF)
(2£ #BLF) remain discernable.

| | | | | | | | Co- |
|---|---|---|---|---|---|---|---|
| | The line | | | | | | existing |
| | number of | | | | | | Regular |
| Group | FIG. 6h | tRNA type | Position | Symbol | Modification | Stoichiometry | Base |
| 1 | 2 | Arg1CU | 58 | ″ | m¹A | 20 ± 6% | A |
| | 5 | AsnGUU | 58 | ″ | m¹A | 18 ± 6% | A |
| | 19 | LysCUU | 9 | L | m²G | 72 ± 16% | G |
| | 23 | ProNGG | 4 | B | Cm | 68 ± 13% | C |
| | 24 | ProNGG | 4 | B | Cm | 68 ± 13% | C |
| 2 | 1 | AlaIGC | | T | m⁵U | 89% | U |
| | 2 | Arg1CU | 54 | T | m⁵U | 98% | U |
| | 4 | ArgICG | 58 | ″ | m¹A | 47% | A |
| | 6 | AspGUC | 54 | T | m⁵U | 98% | U |
| | 9 | Gly.CC | 54 | T | m⁵U | 35% | U |
| | 13 | IleIAU | 9 | K | m¹G | 92% | G |
| | 14 | IniCAU | 58 | ″ | m¹A | 41% | A |
| | 18 | Lys)UU | 58 | ″ | m¹A | 85% | A |
| | 21 | Phe#AA | 49 | ? | m⁵C | 80% | C |
| | 23 | ProNGG | 54 | T | m⁵U | 52% | U |
| | 24 | ProNGG | 54 | T | m⁵U | 52% | U |
| | 28 | ThrIGU | 54 | T | m⁵U | 36% | U |
| | 33 | ValCAC | 10 | L | m²G | 3% | G |

### Mlc-Seq Algorithms' Pseudocodes and Instructions

Pseudocodes and Python source codes for all the algorithms described herein (including homology search, identifying acid-labile nucleotides, MassSum data separation, GapFill, ladder complementation) are listed below and available on GitHub (github.com/rnamodifications/MLC-Seq) under the GPL-3.0 license.
All MS sequence datasets used in the manuscript are publicly available through the corresponding repository on GitHub (github.com/rnamodifications/MLC-Seg).

Homology Search Algorithm

| | |
|---|---|
| Input: | vertex set Vₑₓₚₑᵣᵢₘₑₙₜₐₗ, consists of experimental compounds |
| Output: | List L, consists of tuples and each tuple (v, u, r) represents that compounds v and |

```
u have relationship r
function(Vₑₓₚₑᵣᵢₘₑₙₜₐₗ)
  L ← empty tuple list
  for each v, u in Vₑₓₚₑᵣᵢₘₑₙₜₐₗ do
```

```
if diff(Massᵥ, Massᵤ) ≈ the mass of specific nucleobase or methylation then
  Mass_{specific} ← the specific mass
  push (v, u, Mass_{specific}) into L
end if
```

```
  end for
  return L
end function
```

Acid Labile Algorithm

| | |
|---|---|
| Input: | vertex set V_{noad}, consists of experimental compounds before acid digest |
| | vertex set V_{ad}, consists of experimental compounds after acid digest |
| Output: | List L, consists of tuples and each tuple (v, u, r) represents that compound v |
| | changes to u after acid treatment, and their relationship is r |

```
function(V_{noad}, V_{ad})
  L ← empty tuple list
  for each v in V_{noad} and u in V_{ad} do
```

```
if diff(Massᵥ, Massᵤ) ≈ any acid labile mass difference then
  Mass_{acid}_{_{—}}_{labile} ← the acid labile mass difference
  push (v, u, Mass _{acid}_{_{—}}_{labile}) into L
end if
```

```
  end for
  return L
end function
```

MassSum Algorithm

| | |
|---|---|
| Input: | vertex set Vₑₓₚₑᵣᵢₘₑₙₜₐₗ, consists of experimental compounds |
| | Mass_{intact}, an intact mass |
| Output: | vertex set V, consists of compounds related to the given intact mass |

```
function (Vₑₓₚₑᵣᵢₘₑₙₜₐₗ, Mass_{intact})
  Mass_{H2O} ← initialize with mass value of H2O
  V ← initialize with empty set
  for each v, u in Vₑₓₚₑᵣᵢₘₑₙₜₐₗ do
```

if sum(Massᵥ, Massᵤ) ≈ Mass_{intact} + Mass_{H2O} then
push (v, u) into V
end if

```
  end for
  return L
end function
```

GapFill Algorithm

| | |
|---|---|
| Input: | vertex set Vₑₓₚₑᵣᵢₘₑₙₜₐₗ, consists of experimental compounds |
| | masses tuple (Mass_{left}, Mass_{right}), the masses of both ends |
| Output: | vertex set V, consists of compounds chosen from the gap |

```
function(Vₑₓₚₑᵣᵢₘₑₙₜₐₗ, Mass_{left}, Mass_{right})
  V ← empty vertex set
  for each v in Vₑₓₚₑᵣᵢₘₑₙₜₐₗ do
```

Link_{left} ← diff(Massᵥ, Mass_{left}) ≈ sum(Masses of specific nucleobases)
Link_{right} ← diff(Massᵥ, Mass_{right}) ≈ sum(Masses of specific nucleobases)
If Link_{left} and Link_{right} then
push v into V
end if

```
  end for
  G ← Graph with each node represents a vertex of V
  for each v, u in G do
```

if diff(Massᵥ, Massᵤ) ≈ sum(Masses of specific nucleobases) then
add edge (v, u) into G
end if

```
  end for
  while True do
```

```
Numₘₐₓ ← the max edges count for any nodes of graph G
Nodeᵥ ← the node has the least edges in graph G
Numₘᵢₙ ← the edges count of node Nodeᵥ
if Numₘᵢₙ >= Numₘₐₓ then
  break
else then
  remove Nodeᵥ and its edges from G
end if
```

```
  end while
  V ← vertices for all the remain nodes of G
  return V
end function
```

Ladder Complemention Algorithm

| | |
|---|---|
| Input: | List L_{ᵢₙ}, consists of a tuple (V, Mass_{intact}, d) list, V is a vertex set contains a set of |
| | compounds, or ladder. Mass_{intact} represents the intact mass relative to V, and d is |
| | the ladder direction, etc. from 5′ to 3′ direction, only 3 \| 5 is allowed |
| Output: | List Lₒᵤₜ, consists of a tuple (V, S) list, V represents a mass ladder, and S is the |
| | nucleotide sequence of V |

```
function(Lᵢₙ)
  Lₒᵤₜ ← empty tuple list, and allocate space for at least 76 positions
  for each (V, Mass_{intact}, d) in Lᵢₙ do
```

```
Lₘₐₛₛ ← the mass value list for vertices of V
if d equals 3 then:
  Mass_{H2O} ← the mass value of H2O
  for each mass m in Lₘₐₛₛ do
    m ← Mass_{intact} + Mass_{H2O} − m
  end for
end if
for each mass m in Lₘₐₛₛ do
  p ← int(m / 320.0)
  push m into Lₒᵤₜ at the end of position p
end for
```

for each item pairs (pᵥ, Pᵤ) at adjacent positions in Lₒᵤₜ do

calculate their mass differences and get the relative nucleotide bases

```
  end for
  for each mass ladder in Lₒᵤₜ do
```

V ← the mass ladder
S ← the nucleotide sequence
push (V, S) into Lₒᵤₜ

```
  end for
  return Lₒᵤₜ
end function
```

### MLC-Seq Algorithms

### Description

The implementation of MLC-Seq algorithm to directly sequence RNA.

### System Requirements

### Hardware Requirements

MLC-Seq requires only a standard computer with enough RAM to support the in-memory operations.

### Software Requirements

### OS Requirements

This disclosure supports macOS/Linux and Windows. The project has been tested on the following systems:
macOS: Big Sur (11.1)

### Windows: Windows 10

### Python Dependencies

1. Python version 3.5+ is required. Go to python.org/downloads/to download python and follow the instruction for installation.
2. The other dependencies are listed inside requirements.txt, please refer to the prepare the environment section below on how to install them. They should install in about 2 minutes, depending on your network speed. 433

### Download

Clone this repository. Click the green button “Code” and click the “Download ZIP” button. Now a zip file named “MLC-Seq-main.zip” is downloaded. Unzip this file. In macOS/Linux system, usually the path of the project is ˜/Downloads/MLC-Seq-main. In Windows system, the path of the project is C: \MLC-Seq-main. 439

### Prepare the Environment

Environment preparation for macOS/Linux and Windows will be described separately. 442
Prepare the environment for macOS/Linux

### 1. Open the Terminal.

2. Create a virtual environment by entering the following command
python3-m venv <your_virtual_workspace_path>
To make it simple, we can setup the virtual environment “vir_env” in the “Downloads” folder by
entering the command
python3-m venv ˜/Downloads/vir_env
3. Activate the virtual environment
source <your_virtual_workspace_path>/bin/activate
In this case, enter the command
source ˜/Downloads/vir_env/bin/activate
Go to the root directory of the MLC-Seq project and install all the required libraries. Enter the
command
cd<root_directory_path>
pip install-r requirements.txt
In our case, enter
cd ˜/Downloads/MLC-Seq-main
pip install-r requirements.txt
Prepare the environment for Windows
1. Open the Command Prompt (cmd.exe).
2. Create a virtual environment by entering the following command
python3-m venv <your_virtual_workspace_path>
Similarly, we can setup the virtual environment “vir_env” in C drive by entering the command
python3-m venv C: \vir_env
3. Activate the virtual environment
<your_virtual_workspace_path>\Scripts\activate.bat
In our case, enter the command
C: \vir_env\Scripts\activate.bat
4. Go to the root directory of the project MLC-Seq and install all the required libraries. Enter
the
command
cd<root_directory_path>
pip install-r requirements.txt
In our case, enter
cd C: \MLC-Seq-main
pip install-r requirements.txt
Launch the Project
Run the following command to launch the project, then project MLC-Seq will be open in the default browser.
jupyter notebook
The main page of MLC-Seq is now presented in your default browser. The data is processed in this window.
Project Structure Description
The folder pseudocode includes the pseudo-codes of MLC-Seq algorithms.
The folder modules includes the main tools and algorithms used in the project.
The folder samples has three sub-folders. They are the deconvoluted results (data sources of the algorithms.) for tRNA-Phe, Glu, and Gln.
The folder examples contains several notebooks (.ipnyb) that illustrate the usage of MLC-Seq algorithms in different levels.
The notebook trna_phe_analysis_lite.ipynb is a simplified version of data processing, using one of the isoforms of tRNA-Phe as an example. After further selection (described in the methods section), the result is then used for ladder complementation. The notebook trna_phe_analysis.ipynb presents the data processing using tRNA-Phe as an example.

### REFERENCES

(1) Alfonzo, J. D.; Brown, J. A.; Byers, P. H.; et al. A call for direct sequencing of full-length RNAs to identify all modifications. Nat. Genet. 2021, 53, 1113-1116.
(2) Helm, M.; Motorin, Y. Detecting RNA modifications in the epitranscriptome: predict and validate. Nat. Rev. Genet. 2017, 18, 275-291.
(3) Zheng, G.; Qin, Y.; Clark, W. C.; et al. Efficient and quantitative high-throughput tRNA sequencing. Nat. Methods 2015, 12, 835-837.
(4) Behrens, A.; Rodschinka, G.; Nedialkova, D. D. High-resolution quantitative profiling of tRNA abundance and modification status in eukaryotes by mim-tRNAseq. Mol. Cell 2021, 81, 1802-1815.e7.
(5) Shi, J.; Zhou, T.; Chen, Q. Exploring the expanding universe of small RNAs. Nat. Cell Biol. 2022, 24, 415-423.
(6) Katibah, G. E.; Qin, Y.; Sidote, D. J.; et al. Broad and adaptable RNA structure recognition by the human interferon-induced tetratricopeptide repeat protein IFIT5. Proc. Natl. Acad. Sci. U.S.A. 2014, 111, 12025-12030.
(7) Hauenschild, R.; Tserovski, L.; Schmid, K.; et al. The reverse transcription signature of N-1-methyladenosine in RNA-Seq is sequence dependent. Nucleic Acids Res. 2015, 43, 9950-9964.
(8) Wiener, D.; Schwartz, S. How many tRNAs are out there? Mol. Cell 2021, 81, 1595-1597.
(9) Pratanwanich, P. N.; Yao, F.; Chen, Y.; et al. Identification of differential RNA modifications from nanopore direct RNA sequencing with xPore. Nat. Biotechnol. 2021, 39, 1394-1402.
(10) Begik, O.; Lucas, M. C.; Pryszcz, L. P.; et al. Quantitative profiling of pseudouridylation dynamics in native RNAs with nanopore sequencing. Nat. Biotechnol. 2021, 39, 1278-1291.
(11) Liu, H.; Begik, O.; Lucas, M. C.; et al. Accurate detection of m6A RNA modifications in native RNA sequences. Nat. Commun. 2019, 10, No. 4079.
(12) Parker, M. T.; Knop, K.; Sherwood, A. V.; et al. Nanopore direct RNA sequencing maps the complexity of Arabidopsis mRNA processing and m6A modification. eLife 2020, 9, No. e49658.
(13) Garalde, D. R.; Snell, E. A.; Jachimowicz, D.; et al. Highly parallel direct RNA sequencing on an array of nanopores. Nat. Methods 2018, 15, 201-206.
(14) Thomas, N. K.; Poodari, V. C.; Jain, M.; et al. Direct Nanopore Sequencing of Individual Full Length tRNA Strands. ACS Nano 2021, 15, 16642-16653.
(15) Suzuki, T. The expanding world of tRNA modifications and their disease relevance. Nat. Rev. Mol. Cell Biol. 2021, 22, 375-392.
(16) Su, D.; Chan, C. T. Y.; Gu, C.; et al. Quantitative analysis of ribonucleoside modifications in tRNA by HPLC-coupled mass spectrometry. Nat. Protoc. 2014, 9, 828-841.
(17) Lauman, R.; Garcia, B. A. Unraveling the RNA modification code with mass spectrometry. Mol. Omics 2020, 16, 305-315.
(18) Wetzel, C.; Limbach, P. A. Mass spectrometry of modified RNAs: recent developments. Analyst 2016, 141, 16-23.
(19) Kimura, S.; Dedon, P. C.; Waldor, M. K. Comparative tRNA sequencing and RNA mass spectrometry for surveying tRNA modifications. Nat. Chem. Biol. 2020, 16, 964-972.
(20) Zhang, N.; Shi, S.; Jia, T. Z.; et al. A general LC-MS-based RNA sequencing method for direct analysis of multiple-base modifications in RNA mixtures. Nucleic Acids Res. 2019, 47, No. e125.
(21) Zhang, N.; Shi, S.; Wang, X.; et al. Direct Sequencing of tRNA by 2D-HELS-AA MS Seq Reveals Its Different Isoforms and Dynamic Base Modifications. ACS Chem. Biol. 2020, 15, 1464-1472.
(22) Zhang, N.; Shi, S.; Yuan, X. et al. A General LC-MS-Based Method for Direct and De Novo Sequencing of RNA Mixtures Containing both Canonical and Modified Nucleotides. In RNA Modifications: Methods and Protocols; McMahon, M., Ed.; Springer US: New York, NY, 2021; pp 261-277.
(23) Zhang, N.; Shi, S.; Yoo, B.; et al. 2D-HELS MS Seq: A General LC-MS-Based Method for Direct and de novo Sequencing of RNA Mixtures with Different Nucleotide Modifications. J. Visualized Exp. 2020, 161, No. e61281.
(24) Thomas, B.; Akoulitchev, A. V. Mass spectrometry of RNA. Trends Biochem. Sci. 2006, 31, 173-181.
(25) Yoluç, Y.; Ammann, G.; Barraud, P.; et al. Instrumental analysis of RNA modifications. Crit. Rev. Biochem. Mol. Biol. 2021, 56, 178-204.
(26) Björkbom, A.; Lelyveld, V. S.; Zhang, S.; et al. Bidirectional Direct Sequencing of Noncanonical RNA by Two-Dimensional Analysis of Mass Chromatograms. J. Am. Chem. Soc. 2015, 137, 14430-14438.
(27) Bahr, U.; Aygün, H.; Karas, M. Sequencing of Single and Double Stranded RNA Oligonucleotides by Acid Hydrolysis and MALDI Mass Spectrometry. Anal. Chem. 2009, 81, 3173-3179.
(28) Schimmel, P. The emerging complexity of the tRNA world: mammalian tRNAs beyond protein synthesis. Nat. Rev. Mol. Cell Biol. 2018, 19, 45-58.
(29) Rodriguez, V.; Chen, Y.; Elkahloun, A.; et al. Chromosome 8 BAC array comparative genomic hybridization and expression analysis identify amplification and overexpression of TRMT12 in breast cancer. Genes, Chromosomes Cancer 2007, 46, 694-707.
(30) Wei, F.-Y.; Suzuki, T.; Watanabe, S.; et al. Deficit of tRNALys modification by Cdkal1 causes the development of type 2 diabetes in mice. J. Clin. Invest. 2011, 121, 3598-3608.
(31) Kirchner, S.; Ignatova, Z. Emerging roles of tRNA in adaptive translation, signalling dynamics and disease. Nat. Rev. Genet. 2015, 16, 98-112.
(32) Carell, T.; Brandmayr, C.; Hienzsch, A.; et al. Structure and Function of Noncanonical Nucleobases. Angew. Chem., Int. Ed. 2012, 51, 7110-7131.
(33) Lee, M.; Kim, B.; Kim, V. N. Emerging Roles of RNA Modification: m⁶A and U-Tail. Cell 2014, 158, 980-987.
(34) Klungland, A.; Dahl, J. A. Dynamic RNA modifications in disease. Curr. Opin. Genet. Dev. 2014, 26, 47-52.
(35) Pan, T. N6-methyl-adenosine modification in messenger and long non-coding RNA. Trends Biochem. Sci. 2013, 38, 204-209.
(36) Kim, D.; Lee, J. Y.; Yang, J. S.; et al. The Architecture of SARSCOV-2 Transcriptome. Cell 2020, 181, 914-921.e910.
(37) Jühling, F.; Morl, M.; Hartmann, R. K.; et al. tRNAdb 2009: compilation of tRNA sequences and tRNA genes. Nucleic Acids Res. 2009, 37, D159-D162.
(38) Smardo, F. L.; Calvet, J. P. Sequence analysis of the glutamate tRNA family: evidence for pseudogenes. Gene 1987, 57, 213-220.
(39) Shi, J.; Zhang, Y.; Tan, D.; et al. PANDORA-seq expands the repertoire of regulatory small RNAs by overcoming RNA modifications. Nat. Cell Biol. 2021, 23, 424-436.
(40) Cozen, A. E.; Quartley, E.; Holmes, A. D.; et al. ARM-seq: AlkB-facilitated RNA methylation sequencing reveals a complex landscape of modified tRNA fragments. Nat. Methods 2015, 12, 879-884.
(41) Ohira, T.; Minowa, K.; Sugiyama, K.; et al. Reversible RNA phosphorylation stabilizes tRNA for cellular thermotolerance. Nature 2022, 605 (7909), 372-379.
(42) Zhang, S.; Blain, J. C.; Zielinska, D.; Gryaznov, S. M.; Szostak, J. W. Fast and accurate nonenzymatic copying of an RNA-like synthetic genetic polymer. Proc. Natl. Acad. Sci. U.S.A. 2013, 110 (44), 17732-17737.
(43) Juhling, F.; Morl, M.; Hartmann, R. K.; et al. UCSC tRNA database Ref of tRNA sequences and tRNA genes. Nucleic Acids Res. 2009, 37, D159-162.
(44) Johnson, M.; Zaretskaya, I.; Raytselis, Y.; Merezhuk, Y.; McGinnis, S.; Madden, T. L. NCBI BLAST: a better web interface. Nucleic Acids Res. 2008, 36, W5-W9.
(45) Brenton, A. G.; Godfrey, A. R. Accurate mass measurement: terminology and treatment of data. J. Am. Soc. Mass Spectrom. 2010, 21, 1821-1835.
(46) Drino, A.; Oberbauer, V.; Troger, C.; et al. Production and purification of endogenously modified tRNA-derived small RNAs. RNA Biol. 2020, 17, 1104-1115.
(47) de Crécy-Lagard, V.; Boccaletto, P.; Mangleburg, C. G.; et al. Matching tRNA modifications in humans to their known and predicted enzymes. Nucleic Acids Res. 2019, 47, 2143-2159.

What is claimed:

1. A MLC-Seq platform comprising: (1) de novo sequencing (without sequence input) to read out the full-length tRNA sequences present in an RNA sample, (2) unbiased sequencing of RNA modifications, (3) site-specific mapping of tRNA modifications; and (3) site-specific quantification of partial tRNA modification stoichiometry.

2. The method of claim 1, wherein confirmation of the site-specific mapping of tRNA modifications is performed by cross referencing between tRNA database sequences and the LC-MS data.

3. The method of claim 1, for identifying the sites of partially modified nucleotides by (i) determining in the intact sample, initially observed modifications or editing and (ii) identifying branching thereby providing identity, location and the partially modified nucleotides and confirming the initially observed modifications in the intact sample.

4. A method for de novo sequencing of tRNAs and site-specific quantification of RNA modification stoichiometries wherein said method comprises (i) as a first step starting with a tRNA sample for sequencing and dividing the sample into two samples wherein one half of the sample is referred to as intact and is not subjected to controlled acid hydrolysis while the other half of the sample is subjected to controlled acid hydrolysis; (ii) direct observation of partial nucleotide modifications or editing in the intact RNA sample: (iii) conducting MS ladder sequencing of the RNA sample subjected to controlled acid hydrolysis for de novo base calling of the complete sequence of the tRNA isoforms through data processing that identify and separate each tRNA species or isoform's MS ladders from LC-MS data and wherein if the 5′ and 3′ ladders display sigmoidal curves on a tR-mass plot, said branches in the plot indicate the position and types of partially modified or edited nucleotides; (iv) site-specific quantification of stoichiometry for partial tRNA nucleotide modifications and editing using data from both intact and ladder levels; and (v) EIC peak analysis for determining the stoichiometry ratio of a modification of the tRNA at a given position.

5. The method of claim 2, further comprising ladder level quantification is aligned with relative abundances at the intact level, confirming initially observed modifications or editing.

6. The method of claim 1, wherein said data processing step (iii) is homology searching before, or after, fragmentation of RNA for identification of related RNA isoforms.

7. The method of claim 1, wherein the data processing step (iii) is a MassSum data processing step.

8. The method of claim 1, wherein the data processing step (iii) is a Gap Filling data processing step.

9. The method of claim 1, wherein the data processing step (iii) is a ladder complementation step.

10. The method of claim 1, wherein the data processing (iv) includes the step of identifying acid labile nucleotide modifications by comparing the mass change of intact RNA before and after acid degradation.

11. The method of claim 1 wherein the controlled fragmentation of the RNA is achieved by chemical degradation, enzymatic degradation, or physical degradation.

12. The method of claim 1, wherein the mass measurement is achieved by LC-MS, gas chromatography, capillary electrophoresis, ion mobility spectrometry, or other methods coupled with mass spectrometry.

13. The method of claim 1, further comprising detection of acid labile nucleotide modifications.

14. The method of claim 1, wherein the RNA is treated with AlkB.

15. A kit for use in generating the sequence of one or more RNA molecules and detecting the presence, identity, location, and quantity of RNA nucleotide modifications on said one or more RNA molecules, said kit comprising one or more components for performance of the method of claim 2.

16. The method of claim 1 for use in the monitoring of changes in RNA modification dynamics and map modifications that have altered stoichiometry in different diseases.

17. A method for stoichiometry determination and quantitative mapping of an identified partial RNA modification in an RNA molecule, in a sample comprising a mixture of RNAs, wherein the method includes:
(i) receiving liquid chromatography-mass-spectrometry (LC-MS) data of an RNA sample, where the RNA sample contains partial modified nucleoside resulting in a mass and retention time branch shifting from the non-modified mass-retention time curve, and wherein said RNA is subjected to controlled acid hydrolysis, analyzing the LC-MS data of the RNA;
(ii) filtering the LC-MS data based on mass, the filtering including removing masses smaller than a predetermined size;
(iii) analyzing the filtered LC-MS data, to determine if there is one or more ladder branch in the two dimension mass-retention time plot, analyzing the filtered LC-MS data including:
(a) determining a mass difference between at least two adjacent ladder fragments; and
(b) determining whether the mass difference is equal to at least one of a canonical nucleotide, or a modified nucleotide; and
\textcircled{c} reading-out more than one RNA sequence in parallel, with one containing non-modified RNA canonical nucleotide, the other(s) containing modified or substituted counterpart, or one containing one modified RNA canonical nucleotide, the other(s) containing differently modified or substituted counterpart, as a sequence read after determining no remaining valid nucleotides in the remaining LC-MS data, the RNA sequence including a sequence order of each identified canonical nucleotide and any identified modified nucleotides.
(iv) determining stoichiometry and quantitative mapping the identified partial RNA modifications using relative abundance such as volume and extract ion current of the modification-containing ladder fragments vs the coexisting non-modified counterpart at a given site where the branch starts.

18. A method for site-specific identification of single nucleotide substitutions and/or partial RNA nucleotide modifications in an RNA molecule, in a sample comprising a mixture of RNAs wherein the method includes:
(i) receiving liquid chromatography-mass-spectrometry (LC-MS) data of an RNA sample, where the RNA sample contains modified nucleoside resulting in a mass and retention time branch shifting from the non-modified mass-retention time curve, and wherein said RNA is subjected to controlled acid hydrolysis, analyzing the LC-MS data of the RNA;
(ii) filtering the LC-MS data based on mass, the filtering including removing masses smaller than a predetermined size;
(iii) analyzing the filtered LC-MS data, to determine if there is one or more ladder branch in the two dimension mass-retention time plot, analyzing the filtered LC-MS data including:
(a) determining a mass difference between at least two adjacent ladder fragments; and
(b) determining whether the mass difference is equal to at least one of a canonical nucleotide, or a modified nucleotide; and
(c) reading-out more than one RNA sequence in parallel, with one containing non-modified RNA canonical nucleotide, the other(s) containing modified or substituted counterpart, or one containing one modified RNA canonical nucleotide, the other(s) containing differently modified or substituted counterpart, as a sequence read after determining no remaining valid nucleotides in the remaining LC-MS data, the RNA sequence including a sequence order of each identified canonical nucleotide and any identified modified nucleotides.

19. The method of claim 1, wherein said method is computer implemented.


SEQUENCE LISTING (45 sequences)

SEQ ID NO: 1 - RNA, 76 nt, synthetic construct
  modified_base 10: mod_base = m2g
  modified_base 16: mod_base = dhu
  modified_base 17: mod_base = dhu
  modified_base 26: mod_base = m22g
  modified_base 32: mod_base = cm
  modified_base 34: mod_base = gm
  modified_base 37: mod_base = yw
  modified_base 39: mod_base = p
  modified_base 40: mod_base = m5c
  modified_base 46: mod_base = m7g
  modified_base 49: mod_base = m5c
  modified_base 54: mod_base = m5u
  modified_base 55: mod_base = p
  modified_base 58: mod_base = m1a
  source 1..76: mol_type = other RNA
gcggatttagctcagttgggagagcgccagactgaanatctggaggtcctgtgttcgatccacagaattcgcacca

SEQ ID NO: 2 - RNA, 75 nt, synthetic construct
  modified_base 10: mod_base = m2g
  modified_base 16: mod_base = dhu
  modified_base 17: mod_base = dhu
  modified_base 26: mod_base = m22g
  modified_base 32: mod_base = cm
  modified_base 34: mod_base = gm
  modified_base 37: mod_base = yw
  modified_base 39: mod_base = p
  modified_base 40: mod_base = m5c
  modified_base 46: mod_base = m7g
  modified_base 49: mod_base = m5c
  modified_base 54: mod_base = m5u
  modified_base 55: mod_base = p
  modified_base 58: mod_base = m1a
  source 1..75: mol_type = other RNA
gcggatttagctcagttgggagagcgccagactgaanatctggaggtcctgtgttcgatccacagaattcgcacc

SEQ ID NO: 3 - RNA, 75 nt, synthetic construct
  modified_base 10: mod_base = m2g
  modified_base 16: mod_base = dhu
  modified_base 17: mod_base = dhu
  modified_base 26: mod_base = m22g
  modified_base 32: mod_base = cm
  modified_base 34: mod_base = gm
  modified_base 37: mod_base = yw
  modified_base 39: mod_base = p
  modified_base 40: mod_base = m5c
  modified_base 46: mod_base = m7g
  modified_base 49: mod_base = m5c
  modified_base 54: mod_base = m5u
  modified_base 55: mod_base = p
  modified_base 58: mod_base = m1a
  source 1..75: mol_type = other RNA
gcggatttagctcagttgggagagcgccagactgaanatctggaggtcctgtgttcgatccacagagttcgcacc

SEQ ID NO: 4 - RNA, 76 nt, synthetic construct
  modified_base 10: mod_base = m2g
  modified_base 16: mod_base = dhu
  modified_base 17: mod_base = dhu
  modified_base 26: mod_base = m22g
  modified_base 32: mod_base = cm
  modified_base 34: mod_base = gm
  modified_base 37: mod_base = yw
  modified_base 39: mod_base = p
  modified_base 40: mod_base = m5c
  modified_base 46: mod_base = m7g
  modified_base 49: mod_base = m5c
  modified_base 54: mod_base = m5u
  modified_base 55: mod_base = p
  modified_base 58: mod_base = m1a
  source 1..76: mol_type = other RNA
gcggatttagctcagttgggagagcgccagactgaanatctggaggtcctgtgttcgatccacagagttcgcacca

SEQ ID NO: 5 - RNA, 74 nt, synthetic construct
  modified_base 10: mod_base = m2g
  modified_base 16: mod_base = dhu
  modified_base 17: mod_base = dhu
  modified_base 26: mod_base = m22g
  modified_base 32: mod_base = cm
  modified_base 34: mod_base = gm
  modified_base 37: mod_base = yw
  modified_base 39: mod_base = p
  modified_base 40: mod_base = m5c
  modified_base 46: mod_base = m7g
  modified_base 49: mod_base = m5c
  modified_base 54: mod_base = m5u
  modified_base 55: mod_base = p
  modified_base 58: mod_base = m1a
  source 1..74: mol_type = other RNA
gcggatttagctcagttgggagagcgccagactgaanatctggaggtcctgtgttcgatccacagaattcgcac

SEQ ID NO: 6 - RNA, 75 nt, synthetic construct
  modified_base 6: mod_base = m2g
  modified_base 16: mod_base = dhu
  modified_base 18: mod_base = dhu
  modified_base 19: mod_base = dhu
  modified_base 47: mod_base = m5c
  modified_base 48: mod_base = m5c
  modified_base 52: mod_base = m5u
  modified_base 53: mod_base = um
  modified_base 57: mod_base = m1a
  source 1..75: mol_type = other RNA
tccctggtggtctagtggttaggattcggcgctctcaccgccgcggcccgggttcgattcccggtcagggaacca

SEQ ID NO: 7 - RNA, 76 nt, synthetic construct
  modified_base 9: mod_base = m1g
  modified_base 16: mod_base = dhu
  modified_base 20: mod_base = dhu
  modified_base 26: mod_base = m22g
  modified_base 34: mod_base = OTHER; note = inosine
  modified_base 37: mod_base = m1i
  modified_base 38: mod_base = p
  modified_base 47: mod_base = dhu
  modified_base 54: mod_base = m22g
  modified_base 55: mod_base = p
  source 1..76: mol_type = other RNA
gggcgtgtggcgtagtcggtagcgcgctcccttngcntgggagaggtctccggttcgattccggactcgtccacca

SEQ ID NO: 8 - RNA, 75 nt, synthetic construct
  modified_base 9: mod_base = m1g
  modified_base 10: mod_base = m2g
  modified_base 16: mod_base = dhu
  modified_base 25: mod_base = m22g
  modified_base 26: mod_base = p
  modified_base 33: mod_base = mcm5u
  modified_base 36: mod_base = t6a
  modified_base 38: mod_base = p
  modified_base 46: mod_base = dhu
  modified_base 54: mod_base = m22g
  modified_base 55: mod_base = p
  modified_base 58: mod_base = m1a
  source 1..75: mol_type = other RNA
gctcgcgtggcgtaatggcaacgcgtctgacttctaatcagaagattatgggttcgacccccatcgtgagtgcca

SEQ ID NO: 9 - RNA, 75 nt, synthetic construct
  modified_base 9: mod_base = m1g
  modified_base 10: mod_base = m2g
  modified_base 16: mod_base = dhu
  modified_base 25: mod_base = m22g
  modified_base 26: mod_base = p
  modified_base 33: mod_base = mcm5u
  modified_base 36: mod_base = t6a
  modified_base 38: mod_base = p
  modified_base 46: mod_base = dhu
  modified_base 53: mod_base = m5u
  modified_base 54: mod_base = p
  modified_base 57: mod_base = m1a
  source 1..75: mol_type = other RNA
gcttgcgtggcgtaatggcaacgcgtctgacttctaatcagaagattatgggttcgacccccatcgtgagtgcca

SEQ ID NO: 10 - RNA, 76 nt, synthetic construct
  modified_base 1: mod_base = p
  modified_base 9: mod_base = m1g
  modified_base 10: mod_base = m2g
  modified_base 16: mod_base = dhu
  modified_base 19: mod_base = dhu
  modified_base 26: mod_base = m22g
  modified_base 27: mod_base = p
  modified_base 34: mod_base = OTHER; note = inosine
  modified_base 47: mod_base = dhu
  modified_base 49: mod_base = m5c
  modified_base 54: mod_base = m5u
  modified_base 55: mod_base = p
  modified_base 58: mod_base = m1a
  source 1..76: mol_type = other RNA
ttcctcgtggcccaatggtcacggcgtctggctncgaaccagaagattccaggttcaagtcctggcggggaagcca

SEQ ID NO: 11 - RNA, 77 nt, synthetic construct
  modified_base 10: mod_base = m2g
  modified_base 16: mod_base = dhu
  modified_base 17: mod_base = dhu
  modified_base 20: mod_base = dhu
  modified_base 21: mod_base = dhu
  modified_base 27: mod_base = m22g
  modified_base 38: mod_base = t6a
  modified_base 40: mod_base = p
  modified_base 48: mod_base = dhu
  modified_base 49: mod_base = m5c
  modified_base 55: mod_base = m5u
  modified_base 56: mod_base = p
  modified_base 59: mod_base = m1a
  source 1..77: mol_type = other RNA
gactccatggccaagttggttaaggcgtgcgactgttaatcgcaagatcgtgagttcaaccctcactggggtcgcca

SEQ ID NO: 12 - RNA, 75 nt, synthetic construct
  modified_base 15: mod_base = dhu
  modified_base 18: mod_base = dhu
  modified_base 31: mod_base = p
  modified_base 36: mod_base = i6a
  modified_base 38: mod_base = p
  modified_base 45: mod_base = m7g
  modified_base 46: mod_base = dhu
  modified_base 47: mod_base = m5c
  modified_base 53: mod_base = m5u
  modified_base 54: mod_base = p
  modified_base 57: mod_base = m1a
  source 1..75: mol_type = other RNA
gctcgtatggcgcagtggtagcgcagcagattgcaaatctgttggtccttagttcgatcctgagtgcgagctcca

SEQ ID NO: 13 - RNA, 75 nt, synthetic construct
  modified_base 13: mod_base = p
  modified_base 21: mod_base = dhu
  modified_base 20: mod_base = OTHER; note = u
  modified_base 27: mod_base = p
  modified_base 34: mod_base = mcm5s2u
  modified_base 37: mod_base = OTHER; note = u
  modified_base 48: mod_base = m5c
  modified_base 53: mod_base = m5u
  modified_base 54: mod_base = p
  source 1..75: mol_type = other RNA
tccgatatagtgtaacggctatcacatcacgctttcaccgtggagaccggggttcgactccccgtatcggagcca

SEQ ID NO: 14 - RNA, 74 nt, synthetic construct
  modified_base 13: mod_base = p
  modified_base 16: mod_base = dhu
  modified_base 19: mod_base = dhu
  modified_base 20: mod_base = dhu
  modified_base 52: mod_base = m5u
  modified_base 53: mod_base = p
  source 1..74: mol_type = other RNA
gggcggttagtgtagtggttatcatcccaccctccaaggtggggacacgggttcgattctcgtaccgctcacca

SEQ ID NO: 15 - RNA, 73 nt, synthetic construct
  modified_base 4: mod_base = cm
  modified_base 9: mod_base = m1g
  modified_base 13: mod_base = p
  modified_base 16: mod_base = dhu
  modified_base 19: mod_base = dhu
  modified_base 31: mod_base = p
  modified_base 37: mod_base = p
  modified_base 46: mod_base = m5c
  modified_base 51: mod_base = m5u
  modified_base 52: mod_base = p
  source 1..73: mol_type = other RNA
gcgcaagtggtttagtggtaaaatccaacgttgccatcgttgggccccggttcgattccgggcttgcgcacca

SEQ ID NO: 16 - RNA, 76 nt, synthetic construct
  modified_base 5: mod_base = OTHER; note = am
  modified_base 14: mod_base = p
  modified_base 17: mod_base = dhu
  modified_base 18: mod_base = gm
  modified_base 20: mod_base = dhu
  modified_base 21: mod_base = dhu
  modified_base 33: mod_base = p
  modified_base 38: mod_base = m1g
  modified_base 40: mod_base = p
  modified_base 49: mod_base = m5c
  modified_base 54: mod_base = m5u
  modified_base 55: mod_base = p
  source 1..76: mol_type = other RNA
ggccatcttagtatagtggttagtacacaacattgtggctgttgaaaccctggttcgattctaggaggtggcacca

SEQ ID NO: 17 - RNA, 76 nt, synthetic construct
  modified_base 5: mod_base = OTHER; note = am
  modified_base 14: mod_base = p
  modified_base 17: mod_base = dhu
  modified_base 18: mod_base = gm
  modified_base 20: mod_base = dhu
  modified_base 21: mod_base = dhu
  modified_base 33: mod_base = p
  modified_base 38: mod_base = m1g
  modified_base 49: mod_base = m5c
  modified_base 54: mod_base = m5u
  modified_base 55: mod_base = p
  source 1..76: mol_type = other RNA
ggccatcttagtatagtggttagtacacatcgttgtggccgatgaaaccctggttcgattctaggagatggcacca

SEQ ID NO: 18 - RNA, 77 nt, synthetic construct
  modified_base 9: mod_base = m1g
  modified_base 10: mod_base = m2g
  modified_base 16: mod_base = dhu
  modified_base 17: mod_base = dhu
  modified_base 20: mod_base = dhu
  modified_base 21: mod_base = dhu
  modified_base 35: mod_base = OTHER; note = inosine
  modified_base 38: mod_base = t6a
  modified_base 48: mod_base = dhu
  modified_base 49: mod_base = m5c
  modified_base 55: mod_base = m5u
  modified_base 56: mod_base = p
  modified_base 59: mod_base = m1a
  source 1..77: mol_type = other RNA
ggtctcttggcccagttggttaaggcaccgtgctnataacgcggggatcagcggttcgatcccgctagagaccacca

SEQ ID NO: 19 - RNA, 75 nt, synthetic construct
  modified_base 9: mod_base = m1g
  modified_base 10: mod_base = m2g
  modified_base 16: mod_base = dhu
  modified_base 25: mod_base = m22g
  modified_base 36: mod_base = t6a
  modified_base 45: mod_base = m7g
  modified_base 46: mod_base = dhu
  modified_base 47: mod_base = m5c
  modified_base 48: mod_base = m5c
  modified_base 57: mod_base = m1a
  modified_base 63: mod_base = OTHER; note = Ar
  source 1..75: mol_type = other RNA
agcgccgtggcgcagtggaagcgcgcagggctcataaccctgatgtcctcggatcgaaaccgagcggcgctacca

SEQ ID NO: 20 - RNA, 85 nt, synthetic construct
  modified_base 10: mod_base = m2g
  modified_base 12: mod_base = ac4c
  modified_base 17: mod_base = gm
  modified_base 19: mod_base = dhu
  modified_base 21: mod_base = dhu
  modified_base 27: mod_base = m22g
  modified_base 33: mod_base = p
  modified_base 35: mod_base = m5c
  modified_base 38: mod_base = m1g
  modified_base 40: mod_base = p
  modified_base 57: mod_base = m5c
  modified_base 63: mod_base = m5u
  modified_base 64: mod_base = p
  source 1..85: mol_type = other RNA
ggttgtttggccgagcggtctaaggcgcctgattcaagctcaggtatcgtaagatgcaagagttcgaatctcttagcaaccacca

SEQ ID NO: 21 - RNA, 87 nt, synthetic construct
  modified_base 10: mod_base = m2g
  modified_base 12: mod_base = ac4c
  modified_base 16: mod_base = dhu
  modified_base 17: mod_base = gm
  modified_base 19: mod_base = dhu
  modified_base 21: mod_base = dhu
  modified_base 27: mod_base = m22g
  modified_base 33: mod_base = cm
  modified_base 38: mod_base = m1g
  modified_base 40: mod_base = p
  modified_base 59: mod_base = m5c
  modified_base 65: mod_base = m5u
  modified_base 66: mod_base = p
  modified_base 69: mod_base = m1a
  source 1..87: mol_type = other RNA
ggagggttggccgagtggtctaaggcggcagacttaagatctgttggacggttgtccgcgcgagttcgaacctcgcatccttcacca

SEQ ID NO: 22 - RNA, 85 nt, synthetic construct
  modified_base 10: mod_base = m2g
  modified_base 12: mod_base = ac4c
  modified_base 17: mod_base = gm
  modified_base 19: mod_base = dhu
  modified_base 20: mod_base = dhu
  modified_base 21: mod_base = dhu
  modified_base 27: mod_base = m22g
  modified_base 28: mod_base = p
  modified_base 33: mod_base = p
  modified_base 38: mod_base = m1g
  modified_base 40: mod_base = p
  modified_base 57: mod_base = m5c
  modified_base 63: mod_base = m5u
  modified_base 64: mod_base = p
  modified_base 67: mod_base = m1a
  source 1..85: mol_type = other RNA
gggagtttggccgagtggtttaaggcgtcagatttaggctctgatatcttcggatgcaagggttcgaatcccttagctctcacca

SEQ ID NO: 23 - RNA, 76 nt, synthetic construct
  modified_base 1: mod_base = p
  modified_base 10: mod_base = m2g
  modified_base 16: mod_base = dhu
  modified_base 17: mod_base = dhu
  modified_base 20: mod_base = dhu
  modified_base 26: mod_base = m22g
  modified_base 27: mod_base = p
  modified_base 28: mod_base = p
  modified_base 34: mod_base = OTHER; note = cmnm5um
  modified_base 37: mod_base = t6a
  modified_base 46: mod_base = m7g
  modified_base 47: mod_base = dhu
  modified_base 48: mod_base = m5c
  modified_base 54: mod_base = m5u
  modified_base 55: mod_base = p
  modified_base 58: mod_base = m1a
  modified_base 65: mod_base = p
  source 1..76: mol_type = other RNA
tccttgttagctcagttggtagagcgttcggcttttaaccgaaatgtcaggggttcgagccccctatgaggagcca

SEQ ID NO: 24 - RNA, 76 nt, synthetic construct
  modified_base 9: mod_base = m2g
  modified_base 10: mod_base = m2g
  modified_base 16: mod_base = dhu
  modified_base 20: mod_base = dhu
  modified_base 26: mod_base = m22g
  modified_base 27: mod_base = p
  modified_base 37: mod_base = t6a
  modified_base 39: mod_base = p
  modified_base 46: mod_base = m7g
  modified_base 54: mod_base = m5u
  modified_base 55: mod_base = p
  modified_base 58: mod_base = m1a
  source 1..76: mol_type = other RNA
gccttgttggcgcaatcggtagcgcgtatgactcttaatcataaggttaggggttcgagccccctacagggctcca

SEQ ID NO: 25 - RNA, 76 nt, synthetic construct
  modified_base 10: mod_base = m2g
  modified_base 16: mod_base = dhu
  modified_base 26: mod_base = m22g
  modified_base 27: mod_base = p
  modified_base 31: mod_base = p
  modified_base 37: mod_base = t6a
  modified_base 39: mod_base = p
  modified_base 46: mod_base = m7g
  modified_base 47: mod_base = dhu
  modified_base 48: mod_base = m5c
  modified_base 54: mod_base = m5u
  modified_base 55: mod_base = p
  modified_base 58: mod_base = m1a
  source 1..76: mol_type = other RNA
gcttcagtagctcagtaggaagagcgtcagtctcataatctgaaggtcgagagttcgaacctctcctggagcacca

SEQ ID NO: 26 - RNA, 76 nt, synthetic construct; identical to SEQ ID NO: 1
  modified_base 10: mod_base = m2g
  modified_base 16: mod_base = dhu
  modified_base 17: mod_base = dhu
  modified_base 26: mod_base = m22g
  modified_base 32: mod_base = cm
  modified_base 34: mod_base = gm
  modified_base 39: mod_base = p
  modified_base 40: mod_base = m5c
  modified_base 46: mod_base = m7g
  modified_base 49: mod_base = m5c
  modified_base 54: mod_base = m5u
  modified_base 55: mod_base = p
  modified_base 58: mod_base = m1a
  source 1..76: mol_type = other RNA

SEQ ID NO: 27 - RNA, 76 nt, synthetic construct
  modified_base 10: mod_base = m2g
  modified_base 16: mod_base = dhu
  modified_base 17: mod_base = dhu
  modified_base 26: mod_base = m22g
  modified_base 32: mod_base = cm
  modified_base 34: mod_base = gm
  modified_base 37: mod_base = yw
  modified_base 39: mod_base = p
  modified_base 40: mod_base = m5c
  modified_base 46: mod_base = m7g
  modified_base 49: mod_base = m5c
  modified_base 54: mod_base = m5u
  modified_base 55: mod_base = p
  modified_base 58: mod_base = m1a
  source 1..76: mol_type = other RNA
gcggacttagctcagttgggagagcgccagactgaanatctggaggtcctgtgttcgatccacagagttcgcacca

SEQ ID NO: 28 - RNA, 75 nt, synthetic construct
  modified_base 4: mod_base = cm
  modified_base 9: mod_base = m1g
  modified_base 13: mod_base = p
  modified_base 16: mod_base = dhu
  modified_base 19: mod_base = dhu
  modified_base 31: mod_base = p
  modified_base 33: mod_base = OTHER; note = unknown modified uridine
  modified_base 36: mod_base = m1g
  modified_base 37: mod_base = p
  modified_base 45: mod_base = m7g
  modified_base 53: mod_base = m5u
  modified_base 54: mod_base = p
  modified_base 57: mod_base = m1a
  source 1..75: mol_type = other RNA
gggcgtgtggtctagtggtatgattctcgctttgggtgcgagaggccctgggttcaattcccagctcgcccccca

SEQ ID NO: 29 - RNA, 75 nt, synthetic construct
  modified_base 4: mod_base = cm
  modified_base 13: mod_base = p
  modified_base 16: mod_base = dhu
  modified_base 19: mod_base = dhu
  modified_base 31: mod_base = p
  modified_base 33: mod_base = OTHER; note = inosine
  modified_base 36: mod_base = m1g
  modified_base 37: mod_base = p
  modified_base 45: mod_base = m7g
  modified_base 53: mod_base = m5u
  modified_base 54: mod_base = p
  modified_base 57: mod_base = m1a
  source 1..75: mol_type = other RNA
gggcgtgtggtctagtggtatgattctcgcttngggtgcgagaggccctgggttcaattcccagctcgcccccca

SEQ ID NO: 30 - RNA, 85 nt, synthetic construct
  modified_base 12: mod_base = ac4c
  modified_base 16: mod_base = dhu
  modified_base 17: mod_base = gm
  modified_base 19: mod_base = dhu
  modified_base 20: mod_base = dhu
  modified_base 26: mod_base = m22g
  modified_base 32: mod_base = p
  modified_base 34: mod_base = OTHER; note = inosine
  modified_base 37: mod_base = i6a
  modified_base 39: mod_base = p
  modified_base 44: mod_base = um
  modified_base 57: mod_base = m5c
  modified_base 63: mod_base = m5u
  modified_base 64: mod_base = p
  source 1..85: mol_type = other RNA
ggcaacttggccgagtggttaaggcgaaagattngaaatcttttgggctctgcccgcgcaggttcaaatcctgcagttgtcgcca

SEQ ID NO: 31 - RNA, 84 nt, synthetic construct
  modified_base 12: mod_base = ac4c
  modified_base 16: mod_base = dhu
  modified_base 17: mod_base = gm
  modified_base 19: mod_base = dhu
  modified_base 20: mod_base = dhu
  modified_base 26: mod_base = m22g
  modified_base 32: mod_base = p
  modified_base 34: mod_base = OTHER; note = inosine
  modified_base 37: mod_base = i6a
  modified_base 39: mod_base = p
  modified_base 44: mod_base = um
  modified_base 57: mod_base = m5c
  modified_base 63: mod_base = m5u
  modified_base 64: mod_base = p
  source 1..84: mol_type = other RNA
ggcaacttggccgagtggttaaggcgaaagattngaaatcttttgggctttgcccgcgcaggttcgagtcctgcattgtcgcca

SEQ ID NO: 32 - RNA, 85 nt, synthetic construct
  modified_base 12: mod_base = ac4c
  modified_base 16: mod_base = dhu
  modified_base 17: mod_base = gm
  modified_base 19: mod_base = dhu
  modified_base 20: mod_base = dhu
  modified_base 26: mod_base = m22g
  modified_base 32: mod_base = OTHER; note = unknown modified cytidine
  modified_base 34: mod_base = OTHER; note = unknown modified uridine
  modified_base 37: mod_base = i6a
  modified_base 39: mod_base = p
  modified_base 44: mod_base = um
  modified_base 57: mod_base = m5c
  modified_base 63: mod_base = m5u
  modified_base 64: mod_base = p
  source 1..85: mol_type = other RNA
ggcactatggccgagtggttaaggcgacagacttgaaatctgttgggctctgcccgcgctggttcaaatcctgctggtgtcgcca

SEQ ID NO: 33 - RNA, 76 nt, synthetic construct
  modified_base 10: mod_base = m2g
  modified_base 16: mod_base = dhu
  modified_base 17: mod_base = dhu
  modified_base 20: mod_base = dhu
  modified_base 26: mod_base = m22g
  modified_base 31: mod_base = m3c
  modified_base 34: mod_base = OTHER; note = inosine
  modified_base 37: mod_base = t6a
  modified_base 39: mod_base = p
  modified_base 47: mod_base = dhu
  modified_base 48: mod_base = m5c
  modified_base 54: mod_base = m5u
  modified_base 55: mod_base = p
  modified_base 58: mod_base = m1a
  source 1..76: mol_type = other RNA
gcttctatggccaagttggtaaggcgccacactngtaatgtggagatcatcggttcaaatccgattggaagcacca

SEQ ID NO: 34 - RNA, 76 nt, synthetic construct
  modified_base 10: mod_base = m2g
  modified_base 16: mod_base = dhu
  modified_base 17: mod_base = dhu
  modified_base 20: mod_base = dhu
  modified_base 26: mod_base = m22g
  modified_base 31: mod_base = m3c
  modified_base 34: mod_base = OTHER; note = inosine
  modified_base 37: mod_base = t6a
  modified_base 39: mod_base = p
  modified_base 47: mod_base = dhu
  modified_base 48: mod_base = m5c
  modified_base 54: mod_base = m5u
  modified_base 55: mod_base = p
  modified_base 58: mod_base = m1a
  source 1..76: mol_type = other RNA
gcttctatggccaagttggtaaggcgccacactngtaatgtggagatcgtcggttcaaatccgactggaagcacca

SEQ ID NO: 35 - RNA, 75 nt, synthetic construct
  modified_base 9: mod_base = m1g
  modified_base 10: mod_base = m2g
  modified_base 16: mod_base = dhu
  modified_base 17: mod_base = gm
  modified_base 19: mod_base = dhu
  modified_base 25: mod_base = p
  modified_base 26: mod_base = p
  modified_base 27: mod_base = p
  modified_base 31: mod_base = cm
  modified_base 33: mod_base = cm
  modified_base 38: mod_base = p
  modified_base 45: mod_base = m7g
  modified_base 46: mod_base = dhu
  modified_base 53: mod_base = m5u
  modified_base 54: mod_base = p
  modified_base 57: mod_base = m1a
  modified_base 62: mod_base = p
  source 1..75: mol_type = other RNA
gaagcggtggctcaatggtagagctttcgactccaaatcgaagggttgcaggttcaattcctgtccgtttcacca

SEQ ID NO: 36 - RNA, 78 nt, synthetic construct
  modified_base 10: mod_base = m2g
  modified_base 16: mod_base = dhu
  modified_base 17: mod_base = dhu
  modified_base 18: mod_base = gm
  modified_base 20: mod_base = dhu
  modified_base 21: mod_base = dhu
  modified_base 22: mod_base = dhu
  modified_base 28: mod_base = m2g
  modified_base 37: mod_base = p
  modified_base 39: mod_base = i6a
  modified_base 41: mod_base = p
  modified_base 49: mod_base = dhu
  modified_base 50: mod_base = m5c
  modified_base 56: mod_base = m5u
  modified_base 57: mod_base = p
  modified_base 60: mod_base = m1a
  source 1..78: mol_type = other RNA
ctctcggtagccaagttggtttaaggcgcaagactgtaaatcttgagatcgggcgttcgactcgcccccgggagacca

SEQ ID NO: 37 - RNA, 77 nt, synthetic construct
  modified_base 10: mod_base = m2g
  modified_base 16: mod_base = dhu
  modified_base 20: mod_base = dhu
  modified_base 27: mod_base = m22g
  modified_base 28: mod_base = p
  modified_base 33: mod_base = p
  modified_base 35: mod_base = OTHER; note = ncm5u
  modified_base 48: mod_base = dhu
  modified_base 50: mod_base = m5c
  modified_base 55: mod_base = m5u
  modified_base 56: mod_base = p
  modified_base 59: mod_base = m1a
  source 1..77: mol_type = other RNA
ggtccaatggtccagtggttcaagacgtcgcctttacacggcgaagatcccgagttcgaacctcggttggatcacca

SEQ ID NO: 38 - RNA, 76 nt, synthetic construct
  modified_base 10: mod_base = m2g
  modified_base 13: mod_base = p
  modified_base 20: mod_base = dhu
  modified_base 26: mod_base = m2g
  modified_base 27: mod_base = p
  modified_base 28: mod_base = p
  modified_base 32: mod_base = p
  modified_base 46: mod_base = m7g
  modified_base 47: mod_base = dhu
  modified_base 49: mod_base = m5c
  modified_base 54: mod_base = m5u
  modified_base 55: mod_base = p
  modified_base 58: mod_base = m1a
  source 1..76: mol_type = other RNA
gttccaatagtgtagcggctatcacgttgccttcacacggcaaaggtcccgagttcgatcctcggttggaacacca

SEQ ID NO: 39 - RNA, 76 nt, synthetic construct
  modified_base 9: mod_base = m1g
  modified_base 13: mod_base = p
  modified_base 16: mod_base = dhu
  modified_base 20: mod_base = dhu
  modified_base 21: mod_base = dhu
  modified_base 28: mod_base = p
  modified_base 33: mod_base = p
  modified_base 35: mod_base = OTHER; note = inosine
  modified_base 47: mod_base = m7g
  modified_base 48: mod_base = dhu
  modified_base 49: mod_base = m5c
  modified_base 54: mod_base = m5u
  modified_base 55: mod_base = p
  modified_base 58: mod_base = m1a
  source 1..76: mol_type = other RNA
ggtttcgtggtctagtcggttatggcatctgcttnacacgcagaacgtcccagttcgatcctgggcgaaatcacca

SEQ ID NO: 40 - RNA, 75 nt, synthetic construct
  modified_base 13: mod_base = p
  modified_base 16: mod_base = dhu
  modified_base 19: mod_base = dhu
  modified_base 32: mod_base = p
  modified_base 37: mod_base = m1g
  modified_base 48: mod_base = m5c
  modified_base 53: mod_base = m5u
  modified_base 54: mod_base = p
  source 1..75: mol_type = other RNA
tccgtgatagtttaatggtcagaatgggcgcttgtcgcgtgccagatcggggttcaattccccgtcgcggagcca

SEQ ID NO: 41 - DNA, 22 bp, synthetic construct
  source 1..22: mol_type = other DNA
ctaaccactagaccaccaggga

SEQ ID NO: 42 - DNA, 22 bp, synthetic construct
  source 1..22: mol_type = other DNA
tggaggttccaccgagatttga

SEQ ID NO: 43 - DNA, 18 bp, synthetic construct
  source 1..18: mol_type = other DNA
ctaaccactagaccacca

SEQ ID NO: 44 - DNA, 20 bp, synthetic construct
  source 1..20: mol_type = other DNA
tggaggttccaccgagattt

SEQ ID NO: 45 - RNA, 76 nt, synthetic construct; identical to SEQ ID NO: 1
  modified_base 10: mod_base = m2g
  modified_base 16: mod_base = dhu
  modified_base 17: mod_base = dhu
  modified_base 26: mod_base = m22g
  modified_base 32: mod_base = cm
  modified_base 34: mod_base = gm
  modified_base 37: mod_base = yw
  modified_base 39: mod_base = p
  modified_base 40: mod_base = m5c
  modified_base 46: mod_base = m7g
  modified_base 49: mod_base = m5c
  modified_base 54: mod_base = m5u
  modified_base 55: mod_base = p
  source 1..76: mol_type = other RNA